(19) United States
(12) Patent Application Publication
(10) Pub. No.: US 2024/0196642 A1
(43) Pub. Date: Jun. 13, 2024

(54) ORGANIC COMPOUND, OPTO-ELECTRONIC DEVICE INCLUDING THE SAME, AND ELECTRONIC APPARATUS INCLUDING THE OPTO-ELECTRONIC DEVICE

(71) Applicant: Samsung Display Co., Ltd., Yongin-si (KR)

(72) Inventors: Seran Kim, Yongin-si (KR); Hoilim Kim, Yongin-si (KR); Saerom Park, Yongin-si (KR); Dongsun Yoo, Yongin-si (KR); Junyoung Lee, Yongin-si (KR)

(21) Appl. No.: 18/342,600

(22) Filed: Jun. 27, 2023

(30) Foreign Application Priority Data
Oct. 26, 2022 (KR) 10-2022-0139628

(51) Int. Cl. H10K 50/805; C07D 405/04; C07D 405/10; C07D 409/10; C07D 409/14; C07D 421/04; C07D 421/10; H10K 50/15; H10K 50/16; H10K 85/60

(52) U.S. Cl. CPC H10K 50/805; C07D 405/04; C07D 405/10; C07D 409/10; C07D 409/14; C07D 421/04; C07D 421/10; H10K 50/15; H10K 50/16; H10K 85/636; H10K 85/657

(57) ABSTRACT

An opto-electronic device includes a first electrode, a second electrode facing the first electrode, a photoactive layer arranged between the first electrode and the second electrode, and an organic compound represented by Formula 1:

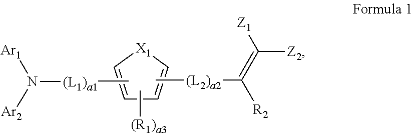

wherein a maximum absorption wavelength of the organic compound is in a range of about 490 nm to about 570 nm, and an oscillator strength (OSC) of the organic compound is in a range of about 0.8 to about 1.0.

### CROSS-REFERENCE TO RELATED APPLICATION

This application claims priority to and the benefit of Korean Patent Application No. 10-2022-0139628, filed on Oct. 26, 2022, in the Korean Intellectual Property Office, the entire content of which is incorporated herein by reference.

### BACKGROUND

### 1. Field

One or more aspects of embodiments of the present disclosure relate to an organic compound, an opto-electronic device including the same, and an electronic apparatus including the opto-electronic device.

### 2. Description of the Related Art

Opto-electronic devices are devices that convert light energy or a light signal into electrical energy or an electrical signal. Examples of opto-electronic devices include a photovoltaic cell and/or a solar cell that converts light energy into electrical energy, a photodetector and/or a light sensor that detects light energy and converts the detected light energy into an electrical signal, and/or the like.

Electronic apparatuses including opto-electronic devices and light-emitting devices have been developed. In an example, light emitted from a light-emitting device may be reflected by an object (e.g., a finger of a user) in contact with an electronic apparatus, to be incident on an opto-electronic device. As the opto-electronic device detects incident light energy and converts the detected incident light energy into an electrical signal, it may be recognized that the object is in contact with the electronic apparatus. The opto-electronic device may be utilized as a fingerprint recognition sensor and/or the like.

The external quantum efficiency (EQE) of an opto-electronic device may be a measure of the ratio of current generated to light absorbed. The dark current density (J_{dark}) of an opto-electronic device represents current generated by heat and/or the like, rather than light, and thus may be a measure of noise in the opto-electronic device. There is a demand for opto-electronic devices having improved photoelectric characteristics such as increased external quantum efficiency and/or decreased dark current density.

### SUMMARY

One or more aspects of embodiments of the present disclosure are directed toward an organic compound having improved characteristics of absorbing light in a set or specific wavelength range, and an opto-electronic device having improved photoelectric characteristics by including the organic compound. One or more aspects of the present embodiments are directed toward a high-quality electronic apparatus including the opto-electronic device.

Additional aspects will be set forth in part in the description which follows and, in part, will be apparent from the description, or may be learned by practice of the presented embodiments of the disclosure.

According to one or more embodiments, an opto-electronic device includes a first electrode, a second electrode facing the first electrode, a photoactive layer arranged between the first electrode and the second electrode, and an organic compound represented by Formula 1:

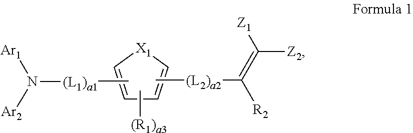

wherein, in Formula 1,
X₁ may be O, S, Se, or Te,
L₁ and L₂ may each independently be a single bond, a C₁-C₂₀ alkylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
a1 and a2 may each independently be an integer from 1 to 5, wherein, when a1 is from 2 to 5, a plurality of L₁(s) may be identical to or different from each other, and when a2 is from 2 to 5, a plurality of L₂(s) may be identical to or different from each other,
R₁ and R₂ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a3 may be an integer from 0 to 2, wherein, when a3 is 2, two R₁(s) may be identical to or different from each other,
Z₁ and Z₂ may each independently be hydrogen, deuterium, —F, —Cl, —Br, —I, —CF₃, —C(═O)OR₃, —SO₃H, or —S(═O)₂R₄,
R₃ and R₄ may each independently be: hydrogen; deuterium; —F; —Cl; —Br; —I; —CF₃; or a C₁-C₂₀ alkyl group substituted with deuterium, —F, —Cl, —Br, —I, —CF₃, or any combination thereof,
when one selected from Z₁ and Z₂ is hydrogen and the other one selected from Z₁ and Z₂ that is not hydrogen is —C(═O)OR₃, R₃ may not be hydrogen,
Ar₁ and Ar₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
Ar₁ and Ar₂ may optionally be bonded to each other via a single bond,
R₁₀ₐ may be:
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group, or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments, an electronic apparatus includes the opto-electronic device.

According to one or more embodiments, provided is the organic compound represented by Formula 1.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic view of an opto-electronic device according to one or more embodiments;

FIG. 2 is a schematic view of a light-emitting device included in an electronic apparatus according to one or more embodiments;

FIG. 3 is a schematic view of an opto-electronic device according to one or more embodiments;

FIG. 4 is a schematic view of an opto-electronic device according to one or more embodiments;

FIG. 5 is a schematic view of an electronic apparatus according to one or more embodiments;

FIG. 6 is a schematic view of an electronic apparatus according to one or more embodiments;

FIG. 7 is a schematic perspective view of an electronic device including an opto-electronic device according to one or more embodiments;

FIG. 8 is a schematic view of the exterior of a vehicle as an electronic device including an opto-electronic device according to one or more embodiments; and

FIGS. 9A-9C are schematic views each being of an interior of a vehicle according to one or more embodiments.

### DETAILED DESCRIPTION

Reference will now be made in more detail to embodiments, examples of which are illustrated in the accompanying drawings, wherein like reference numerals refer to like elements throughout, and duplicative descriptions thereof may not be provided. In this regard, the present embodiments may have different forms and should not be construed as being limited to the descriptions set forth herein. Accordingly, the embodiments are merely described below, by referring to the drawings, to explain aspects of the present description. As utilized herein, the term “and/or” includes any and all combinations of one or more of the associated listed items. As used herein, expressions such as “at least one of”, “one of”, and “selected from”, when preceding a list of elements, modify the entire list of elements and do not modify the individual elements of the list. For example, throughout the disclosure, the expression “at least one of a, b or c”, “at least one selected from a, b and c”, “at least one selected from among a, b and c”, “at least one of a to c”, “at least one selected from a to c”, and “at least one of a, b and/or c” indicates only a, only b, only c, both (e.g., simultaneously) a and b, both (e.g., simultaneously) a and c, both (e.g., simultaneously) b and c, all of a, b, and c, or variations thereof. Further, the use of “may” when describing embodiments of the present disclosure refers to “one or more embodiments of the present disclosure”.

It will be understood that, although the terms first, second, etc. may be used herein to describe various elements, these elements should not be limited by these terms. These terms are only used to distinguish one element from another element. Thus, a first element could be termed a second element without departing from the teachings of the present invention. Similarly, a second element could be termed a first element. As used herein, the singular forms are intended to include the plural forms as well, unless the context clearly indicates otherwise.

As used herein, the singular forms “a,” “an,” and “the” are intended to include the plural forms as well, unless the context clearly indicates otherwise.

It will be further understood that the terms “includes,” “including,” “comprises,” and/or “comprising,” when used in this specification, specify the presence of stated features, steps, operations, elements, and/or components, but do not preclude the presence or addition of one or more other features, steps, operations, elements, components, and/or groups thereof.

It will be understood that when an element is referred to as being “on,” “connected to,” or “coupled to” another element, it may be directly on, connected, or coupled to the other element or one or more intervening elements may also be present. When an element is referred to as being “directly on,” “directly connected to,” or “directly coupled to” another element, there are no intervening elements present.

Spatially relative terms, such as “beneath,” “below,” “lower,” “above,” “upper,” “bottom,” “top” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

As used herein, the terms “substantially”, “about”, and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The electronic device and/or any other relevant devices or components according to embodiments of the present invention described herein may be implemented utilizing any suitable hardware, firmware (e.g. an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure

One or more embodiments of the disclosure provide an opto-electronic device including a first electrode, a second electrode facing the first electrode, a photoactive layer arranged between the first electrode and the second electrode, and an organic compound represented by Formula 1. Formula 1 will be described in more detail herein below.

In one or more embodiments, the first electrode may be an anode. The second electrode may be a cathode. The photoactive layer may absorb light to generate excitons. The excitons may be separated into electrons and holes. As the electrons and holes are generated, current may flow.

In one or more embodiments, the photoactive layer may include the organic compound. The organic compound may absorb light to generate electrons and holes, and may serve as an electron donor. The organic compound may be referred to as an electron donating compound.

In one or more embodiments, the opto-electronic device may further include an electron accepting compound. The electron accepting compound may serve as an electron acceptor. For example, the electron accepting compound may be fullerene, or may be represented by one of Formulae 40-1 to 40-4:

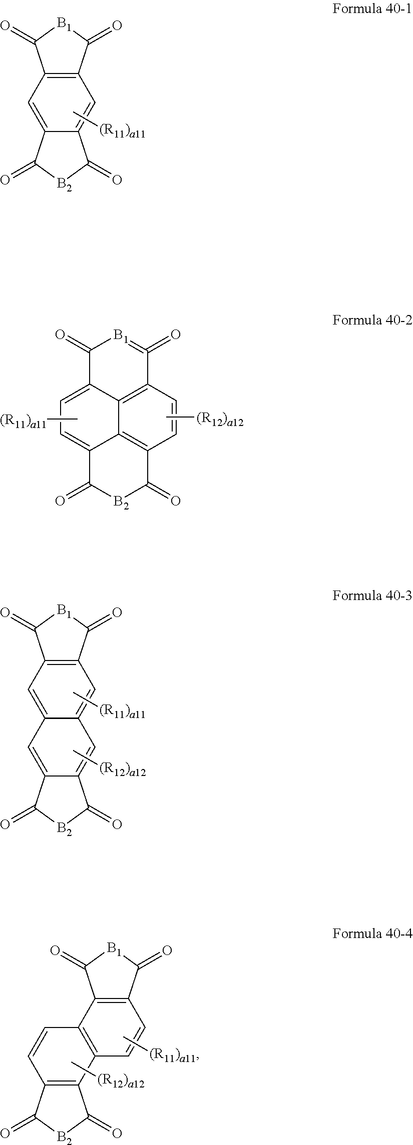

wherein, in Formulae 40-1 to 40-4,
B₁ may be O or N(Ar₁₁),
B₂ may be O or N(Ar₁₂),
Ar₁₁ and Ar₁₂ may each independently be hydrogen, deuterium, a C₁-C₆₀ alkyl group unsubstituted or substituted with at least one R_{10b}, a C₃-C₆₀ carbocyclic group unsubstituted or substituted with at least one R_{10b}, or a C₁-C₆₀ heterocyclic group unsubstituted or substituted with at least one R_{10b},
a11 and a12 may each independently be 0, 1, or 2,
R¹¹ and R¹² may each independently be hydrogen, deuterium, —F, —Cl, —Br, —I, a hydroxyl group, a cyano group, a nitro group, a C₁-C₆₀ alkyl group unsubstituted or substituted with at least one R_{10b}, a C₂-C₆₀ alkenyl group unsubstituted or substituted with at least one R_{10b}, a C₂-C₆₀ alkynyl group unsubstituted or substituted with at least one R_{10b}, a C₁-C₆₀ alkoxy group unsubstituted or substituted with at least one R_{10b}, a C₃-C₆₀ carbocyclic group unsubstituted or substituted with at least one R_{10b}, a C₁-C₆₀ heterocyclic group unsubstituted or substituted with at least one R_{10b}, a C₆-C₆₀ aryloxy group unsubstituted or substituted with at least one R_{10b}, a C₆-C₆₀ arylthio group unsubstituted or substituted with at least one R_{10b}, a C₇-C₆₀ arylalkyl group unsubstituted or substituted with at least one R_{10b}, a C₂-C₆₀ heteroarylalkyl group unsubstituted or substituted with at least one R_{10b}, —C(Q₅₁)(Q₅₂)(Q₅₃), —Si(Q₅₁)(Q₅₂)(Q₅₃), —N(Q₅₁)(Q₅₂), —B(Q₅₁)(Q₅₂), —C(═O)(Q₅₁), —S(═O)₂(Q₅₁), or —P(═O)(Q₅₁)(Q₅₂),
R_{10b} may be as defined herein in connection with R₁₀ₐ, and
Q₅₁ to Q₅₃ may each independently be as defined herein in connection with Q₁.

In one or more embodiments, the electron accepting compound may be fullerene. For example, the electron accepting compound may be fullerene 60 or fullerene 70:

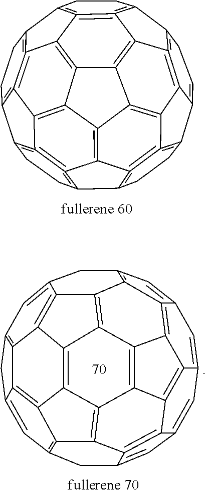

In one or more embodiments, the electron accepting compound may be one of Compounds N1 to N36:

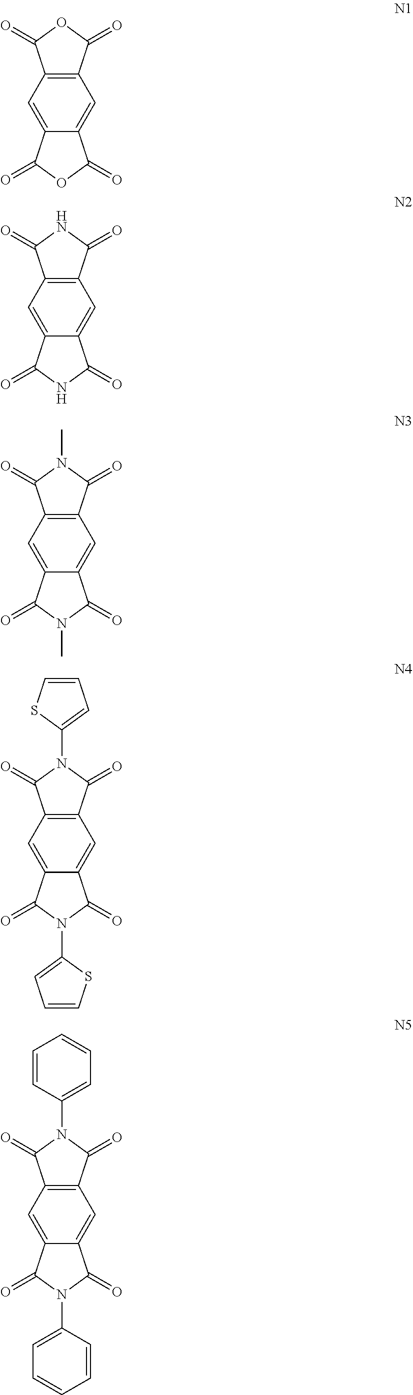

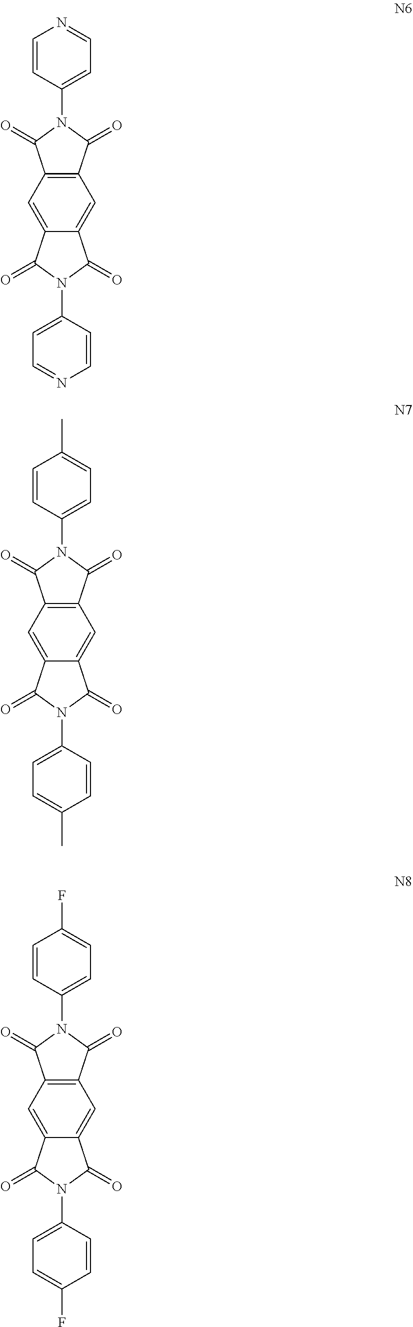

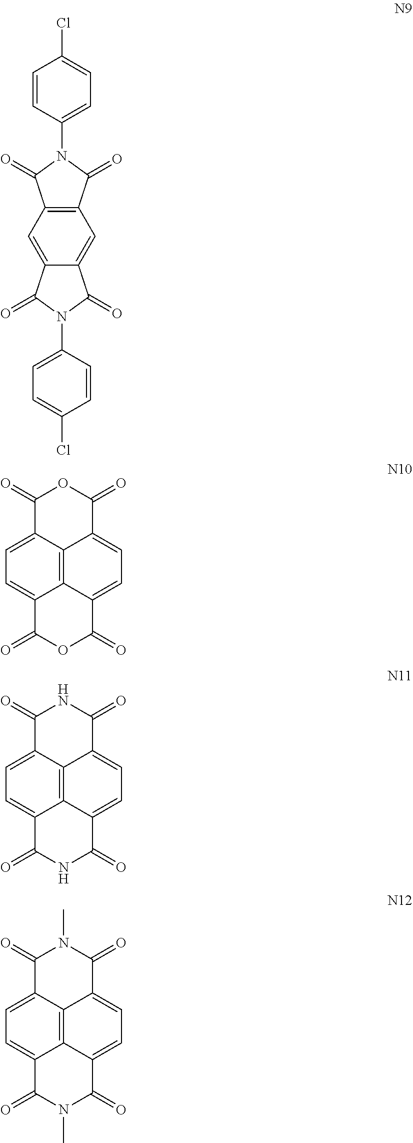

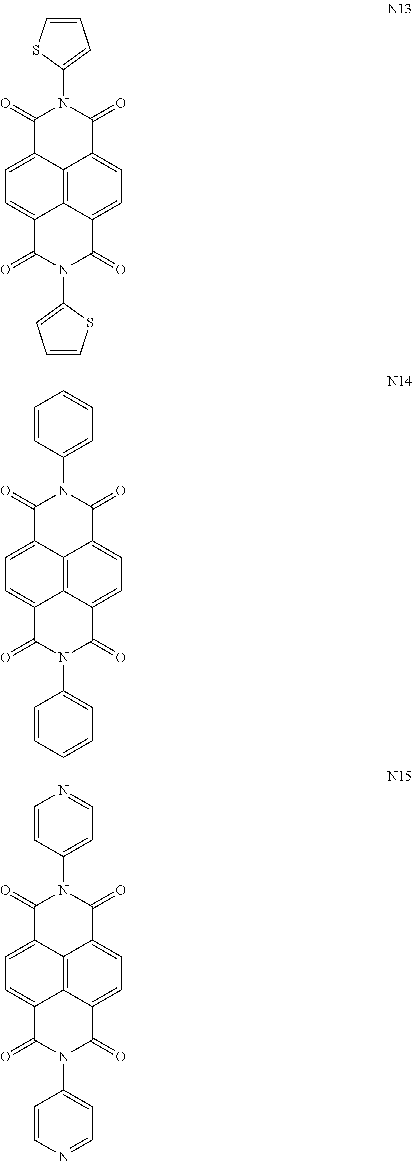

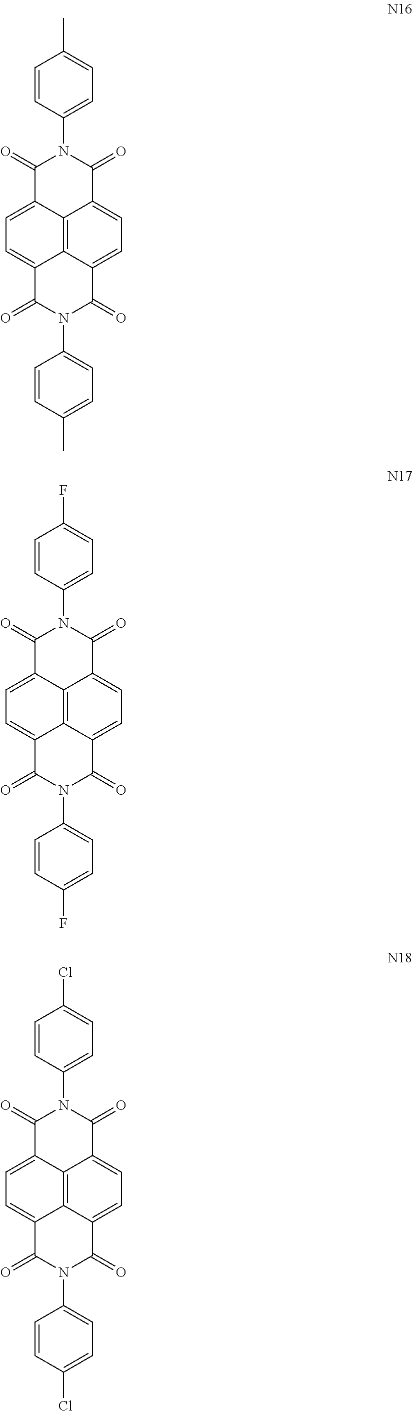

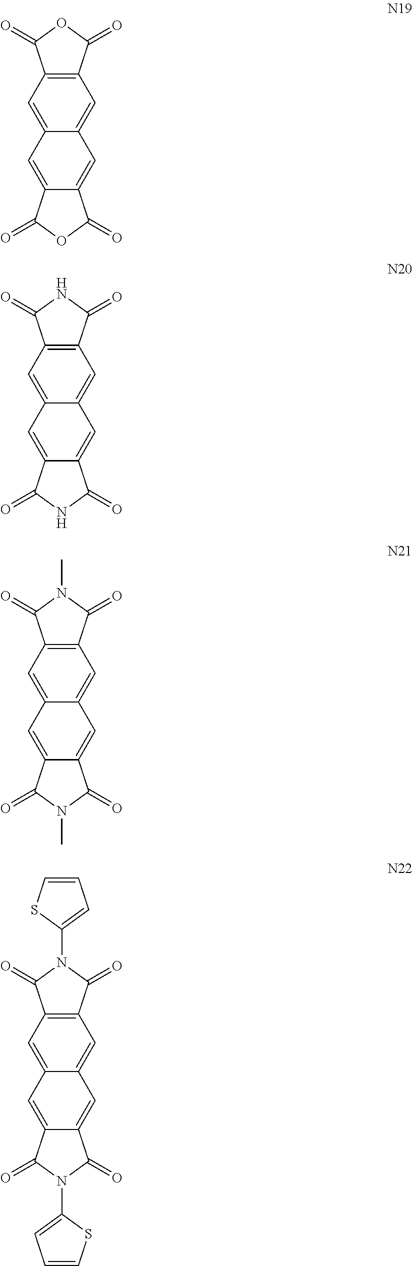

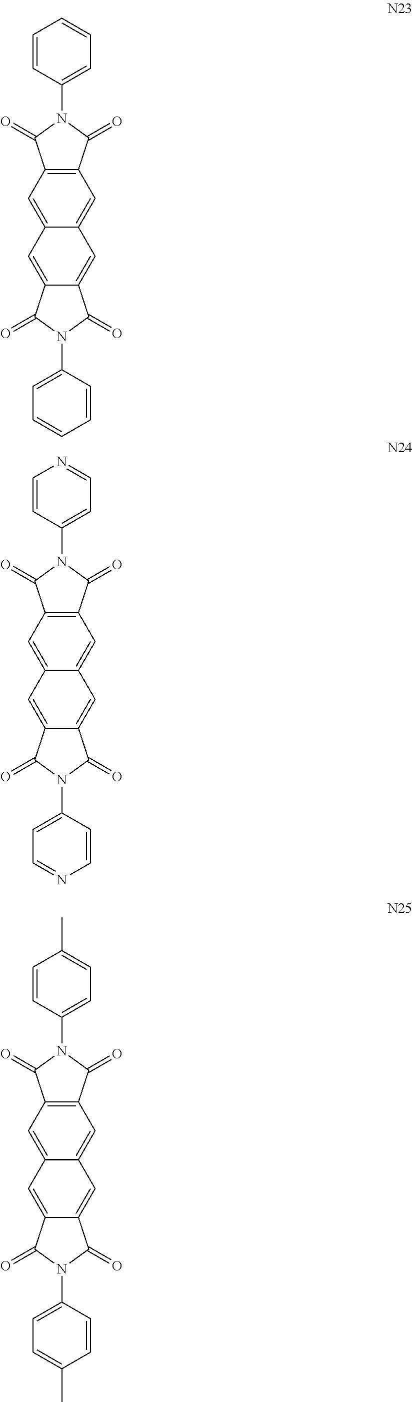

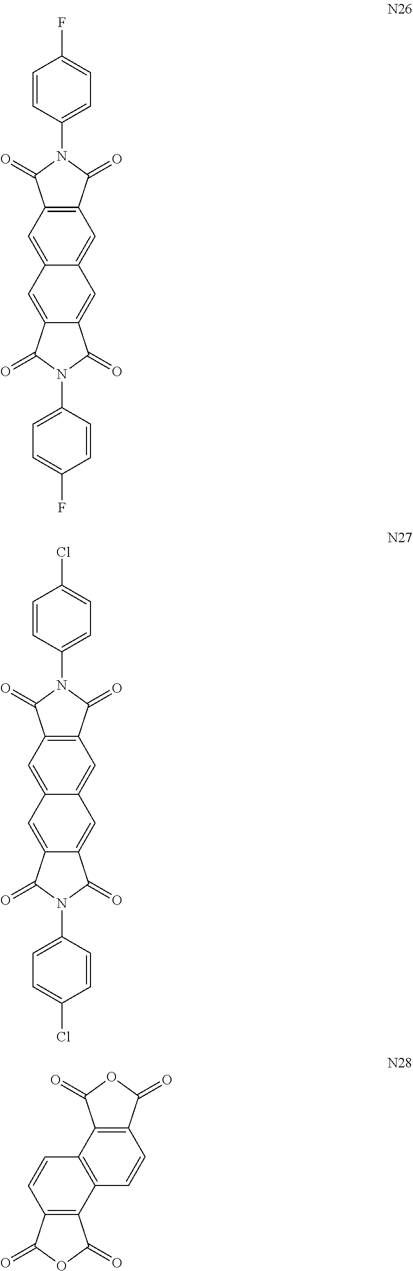

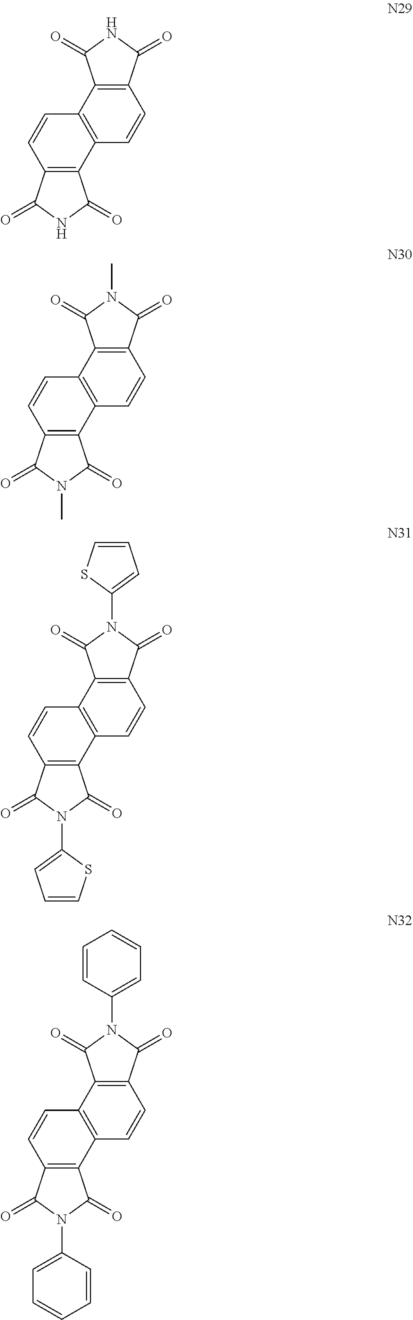

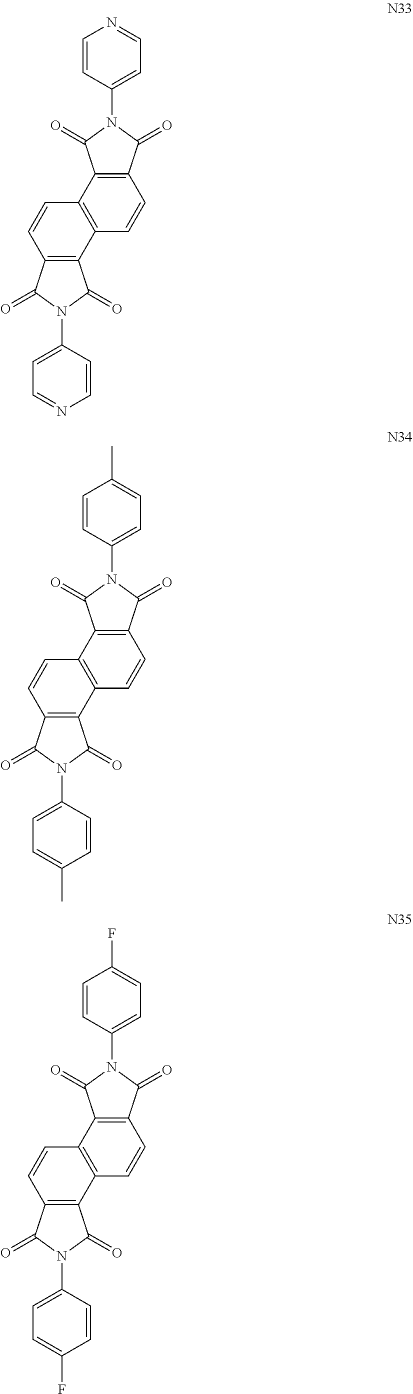 [SMILES: O=c1c2ccc3c(ccc4c(=O)n(-c5ccncc5)c(=O)c43)c2c(=O)n1-c1ccncc1 ; Cc1ccc(-n2c(=O)c3ccc4c(ccc5c(=O)n(-c6ccc(C)cc6)c(=O)c54)c3c2=O)cc1 ; O=c1c2ccc3c(ccc4c(=O)n(-c5ccc(F)cc5)c(=O)c43)c2c(=O)n1-c1ccc(F)cc1]

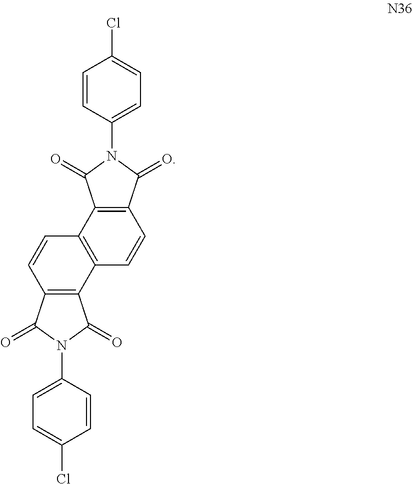 [SMILES: O=c1c2ccc3c(ccc4c(=O)n(-c5ccc(Cl)cc5)c(=O)c43)c2c(=O)n1-c1ccc(Cl)cc1]

In one or more embodiments, the photoactive layer may include the organic compound and the electron accepting compound.

In one or more embodiments, the opto-electronic device may further include a hole transport region arranged between the first electrode and the photoactive layer, and an electron transport region arranged between the photoactive layer and the second electrode. Holes generated by the photoactive layer may move to the first electrode through the hole transport region. Electrons generated by the photoactive layer may move to the second electrode through the electron transport region.

In one or more embodiments, the photoactive layer may include a first layer adjacent to the hole transport region and a second layer adjacent to the electron transport region.

In one or more embodiments, the first layer may include the organic compound. For example, the first layer may not include (e.g., may exclude) the electron accepting compound.

In one or more embodiments, the second layer may include the electron accepting compound. The electron accepting compound may be fullerene. For example, the second layer may not include (e.g., may exclude) the organic compound.

The first layer may be referred to as a p-type photoactive layer, and the second layer may be referred to as an n-type photoactive layer.

In one or more embodiments, the photoactive layer may further include a third layer arranged between the first layer and the second layer. The third layer may include the organic compound and the electron accepting compound.

One or more embodiments of the disclosure provide an electronic apparatus including the opto-electronic device.

In one or more embodiments, the electronic apparatus may further include a light-emitting device adjacent to the opto-electronic device. The light-emitting device may not overlap the opto-electronic device.

For example, light emitted by the light-emitting device may be extracted to the outside of the electronic apparatus. The light may be reflected by an external object to be incident into the electronic apparatus. The opto-electronic device may absorb the incident light. For example, the opto-electronic device may be utilized as a sensor that recognizes an object outside the electronic apparatus.

In one or more embodiments, the light-emitting device may include a first electrode, a second electrode facing the first electrode, and an emission layer arranged between the first electrode and the second electrode. In one or more embodiments, the first electrode of the light-emitting device may be a portion of the first electrode of the opto-electronic device. In one or more embodiments, the first electrode of the light-emitting device may be apart from (e.g., may be spaced from) the first electrode of the opto-electronic device, but may include the same material as the first electrode of the opto-electronic device. In one or more embodiments, the second electrode of the light-emitting device may be a portion of the second electrode of the opto-electronic device. In one or more embodiments, the second electrode of the light-emitting device may be apart from (e.g., may be spaced from) the second electrode of the opto-electronic device, but may include the same material as the second electrode of the opto-electronic device. The emission layer may include a dopant and a host, and may emit light.

In one or more embodiments, the opto-electronic device may further include a hole transport region arranged between the first electrode and the emission layer, and an electron transport region arranged between the emission layer and the second electrode. The hole transport region may include a hole injection layer, a hole transport layer, an emission auxiliary layer, an electron blocking layer, or any combination thereof. The electron transport region may include a buffer layer, a hole blocking layer, an electron control layer, an electron transport layer, an electron injection layer, or any combination thereof. For example, the emission auxiliary layer may generate a resonance phenomenon by light interference, and the buffer layer may control injection of electrons and prevent or reduce loss of holes.

For example, the first electrode, the hole transport region, the electron transport region, and the second electrode included in the opto-electronic device may be substantially identical to or different from the first electrode, the hole transport region, the electron transport region, and the second electrode included in the light-emitting device, respectively. For example, a portion of the opto-electronic device may extend to constitute a portion of the light-emitting device.

In one or more embodiments, the electronic apparatus may further include a thin-film transistor, a color filter, a color conversion layer, a touch screen layer, a polarizing layer, or any combination thereof, the thin-film transistor being electrically connected to the first electrode.

One or more embodiments of the disclosure provide an electronic device including the electronic apparatus. The electronic device may be selected from a flat panel display, a curved display, a computer monitor, a medical monitor, a television, a billboard, an indoor and/or outdoor lighting and/or signal light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a three-dimensional (3D) display, a virtual and/or augmented-reality display, a vehicle, a video wall including multiple displays tiled together, a theater and/or stadium screen, a phototherapy device, and a signboard.

One or more embodiments of the disclosure provide an organic compound represented by Formula 1:

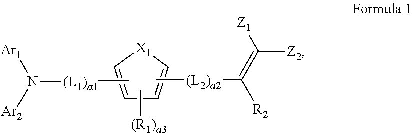

wherein, in Formula 1,
X₁ may be O, S, Se, or Te,
L₁ and L₂ may each independently be a single bond, a C₁-C₂₀ alkylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
a1 and a2 may each independently be an integer from 1 to 5, wherein, when a1 is from 2 to 5, a plurality of L₁(s) may be identical to or different from each other, and when a2 is from 2 to 5, a plurality of L₂(s) may be identical to or different from each other,
R₁ and R₂ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a3 may be an integer from 0 to 2, wherein, when a3 is 2, two R₁(s) may be identical to or different from each other,
Z₁ and Z₂ may each independently be: hydrogen; deuterium; —F; —Cl; —Br; —I; —CF₃; —C(═O)OR₃; —SO₃H; or —S(═O)₂R₄,
R₃ and R₄ may each independently be: hydrogen; deuterium; —F; —Cl; —Br; —I; —CF₃; or a C₁-C₂₀ alkyl group substituted with deuterium, —F, —Cl, —Br, —I, —CF₃, or any combination thereof,
when one selected from Z₁ and Z₂ is hydrogen and the other one selected from Z₁ and Z₂ that is not hydrogen is —C(═O)OR₃, R₃ may not be hydrogen,
Ar₁ and Ar₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
Ar₁ and Ar₂ may optionally be bonded to each other via a single bond,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

In one or more embodiments, the organic compound may be represented by one of Formulae 1-1 to 1-4:

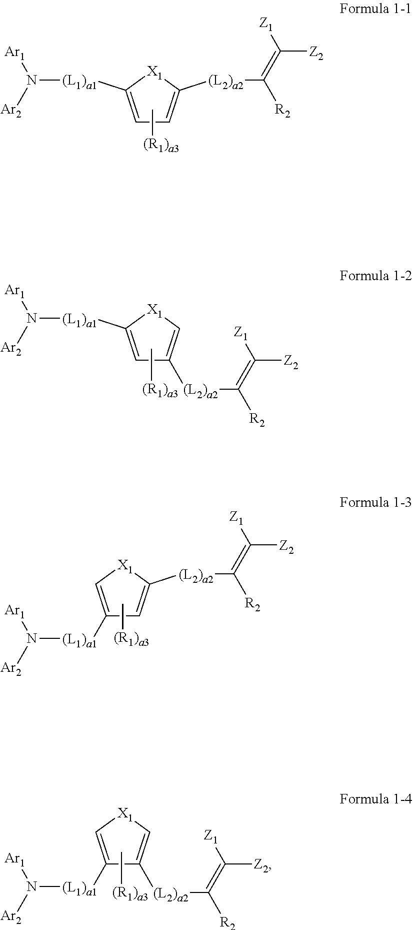

wherein, in Formulae 1-1 to 1-4,
X₁, L₁, L₂, a1 to a3, R₁, R₂, Z₁, Z₂, Ar₁, Ar₂, and R₁₀ₐ may each be as defined herein.

In one or more embodiments, L₁ and L₂ may each independently be selected from a single bond and groups represented b Formulae 2-1 to 2-3:

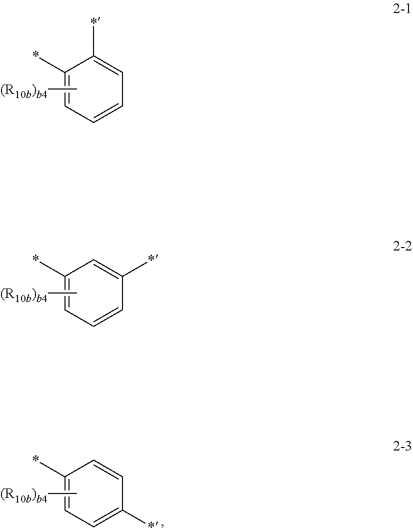

wherein, in Formulae 2-1 to 2-3,
R_{10b} may be as defined herein in connection with R₁₀ₐ,
b4 may be an integer from 0 to 4,
* may indicate a binding site to N in Formula 1, and
*′ may indicate a binding site to a corresponding neighboring atom in Formula 1.

In one or more embodiments, R_{10b} may be deuterium, or b4 may be 0. i) In one or more embodiments, R_{10b} may be deuterium, and b4 may not be 0. ii) In one or more embodiments, R_{10b} may not be deuterium, and b4 may be 0. iii) In one or more embodiments, R_{10b} may be deuterium, and b4 may be 0.

In one or more embodiments, L₂ may be a single bond.

In one or more embodiments, the sum of the number of oxygen atoms included in Z₁ and the number of oxygen atoms included in Z₂ may be 4. In one or more embodiments, Z₁ may be —C(═O)OR₃, and Z₂ may be —S(═O)₂R₄. In one or more embodiments, Z₁ may be —S(═O)₂R₄, and Z₂ may be —C(═O)OR₃. In one or more embodiments, Z₁ and Z₂ may both (e.g., simultaneously) be —C(═O)OR₃. In one or more embodiments, Z₁ and Z₂ may both (e.g., simultaneously) be —S(═O)₂R₄.

In one or more embodiments, Z₁ and Z₂ may not each include (e.g., may each exclude) a nitrogen atom. For example, Z₁ and Z₂ may not each include (e.g., may each exclude) a cyano group.

In one or more embodiments, Z₁ and Z₂ may each independently be: —C(═O)OR₃; or —S(═O)₂R₄, and R₃ and R₄ may each independently be: —F; —Cl; —Br; —I; or a C₁-C₁₀ alkyl group substituted with deuterium, —F, —Cl, —Br, —I, —CF₃, or any combination thereof.

In one or more embodiments, Z₁ and Z₂ may each independently be —C(═O)OCH₃, —C(═O)OCH₂CH₃, or —SO₂Cl.

In one or more embodiments, at least one selected from Z₁ and Z₂ may be —C(═O)OCH₂CH₃ or —SO₂Cl.

In one or more embodiments, Ar₁ may be a benzene group unsubstituted or substituted with at least one R_{1c}, and Ar₂ may be a benzene group unsubstituted or substituted with at least one R_{2c}, wherein R_{1c} and R_{2c} may each be as defined herein in connection with R₁₀ₐ.

In one or more embodiments, R_{1c} and R_{2c} may each independently be: deuterium; a C₁-C₁₀ alkyl group; a C₁-C₁₀ alkoxy group; a phenyl group; a carbazolyl group; or any combination thereof; or a phenyl group or a carbazolyl group, each substituted with deuterium, a C₁-C₁₀ alkyl group, a C₁-C₁₀ alkoxy group, a phenyl group, a carbazolyl group, or any combination thereof.

In one or more embodiments,
the organic compound may be represented by one of Formulae 3-1 to 3-5:

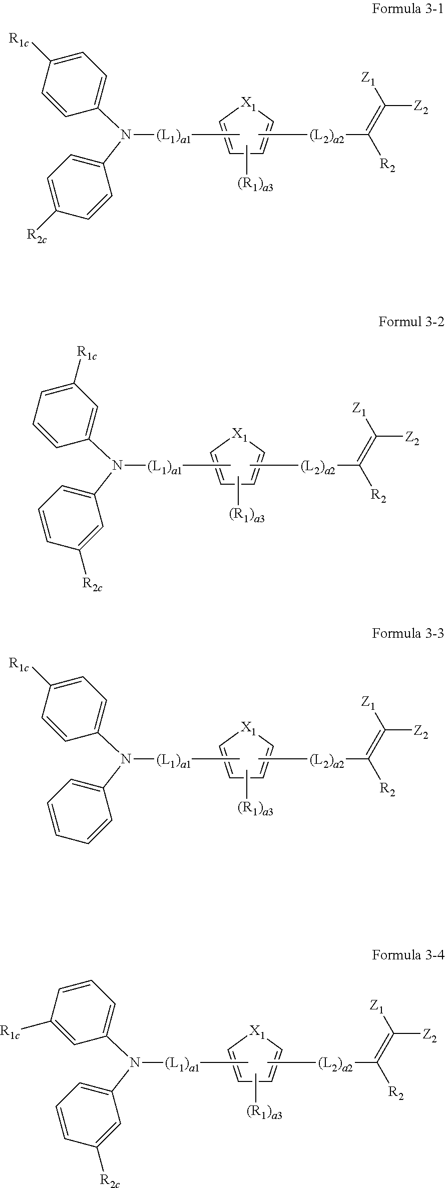

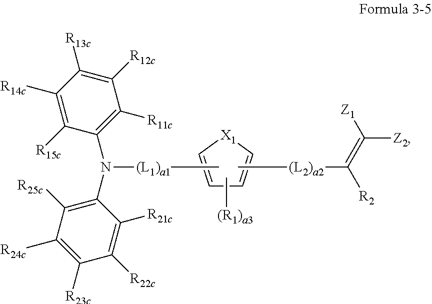

wherein, in Formulae 3-1 to 3-5,
X₁, L₁, L₂, a1 to a3, R₁, R₂, Z₁, and Z₂ may each be as defined herein, and R_{1c}, R_{2c}, R_{11c} to R_{15c}, and R_{21c} to R_{25c} may each be as defined herein in connection with R₁₀ₐ.

In one or more embodiments, in Formula 3-5, at least two of R₁₁ to R_{15c} may each be deuterium, and at least two of R_{21c} to R_{25c} may each be deuterium. In one or more embodiments, R_{11c}, R_{12c}, R_{14c}, and R_{15c} may each be deuterium, and R_{21c}, R_{22c}, R_{24c}, and R_{25c} may each be deuterium. In one or more embodiments, R_{11c} to R_{15c} and R_{21c} to R_{25c} may all be deuterium.

In one or more embodiments,
the organic compound may be represented by one of Formulae 4-1 to 4-4:

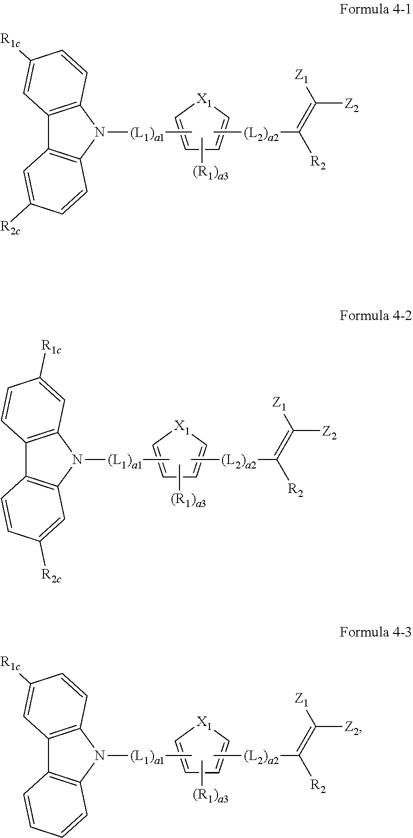

wherein, in Formulae 4-1 to 4-3,
X₁, L₁, L₂, a1 to a3, R₁, R₂, Z₁, and Z₂ may each be as defined herein, and
R_{1c}, R_{2c}, R_{11c} to R_{15c}, and R_{21c} to R_{25c} may each be as defined herein in connection with R₁₀ₐ.

For example, Ar₁ and Ar₂ in Formula 1 may both (e.g., simultaneously) be a benzene group, and Ar₁ and Ar₂ may be bonded to each other via a single bond. For example, -[(L₁)ₐ₁] may be linked to a carbazole group unsubstituted or substituted with at least one substituent.

In one or more embodiments, a maximum absorption wavelength of the organic compound may be in a range of about 490 nm to about 570 nm. For example, the organic compound may absorb green light. In some embodiments, the maximum absorption wavelength of the organic compound may be in a range of about 520 nm to about 540 nm. For example, among the light emitted by the light-emitting device, green light having relatively high luminescence efficiency may be absorbed by the organic compound. Accordingly, the opto-electronic device including the organic compound may be efficiently or suitably utilized as a sensor and/or the like.

In one or more embodiments, a highest occupied molecular orbital (HOMO) energy level of the organic compound may be in a range of about −6.0 eV to about −5.0 eV. For example, the HOMO energy level of the organic compound may be in a range of about −5.6 eV to about −5.4 eV.

In one or more embodiments, a lowest unoccupied molecular orbital (LUMO) energy level of the organic compound may be in a range of about −3.5 eV to about −2.5 eV. For example, the LUMO energy level of the organic compound may be in a range of about −3.0 eV to about −2.8 eV.

In one or more embodiments, an exciton binding energy of the organic compound may be about 0.23 eV or less. Accordingly, the opto-electronic device including the organic compound may have excellent or suitable characteristics in terms of absorbing light and separating the absorbed light into electrons and holes.

In one or more embodiments, an oscillator strength (OSC) of the organic compound may be in a range of about 0.8 to about 1.0. For example, the OSC of the organic compound may be in a range of about 0.8 to about 0.9. Accordingly, the opto-electronic device including the organic compound may have excellent or suitable light absorption characteristics.

For example, the organic compound may have improved photoelectric characteristics.

In one or more embodiments, the organic compound may be one of Compounds 1 to 40:

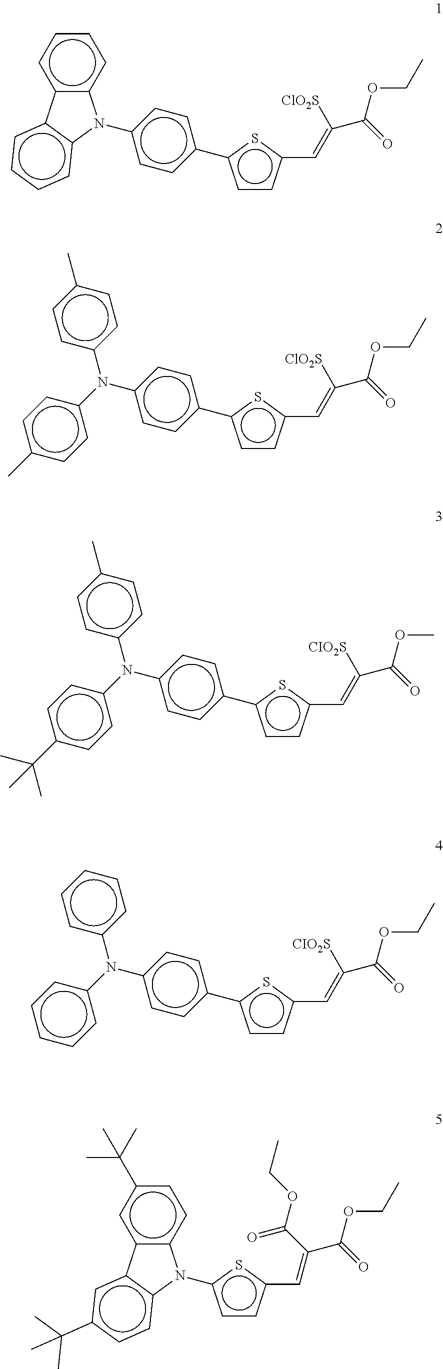

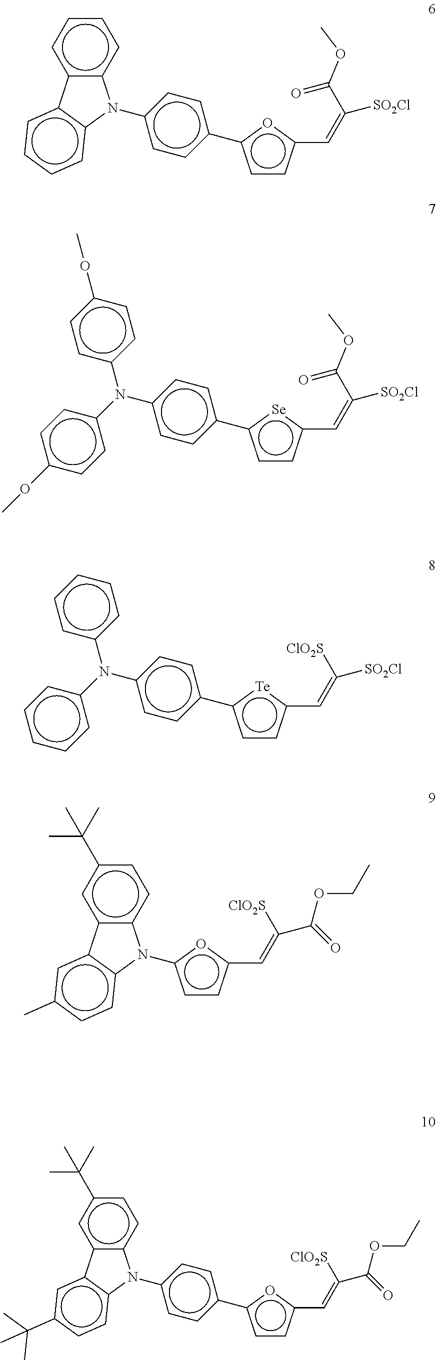

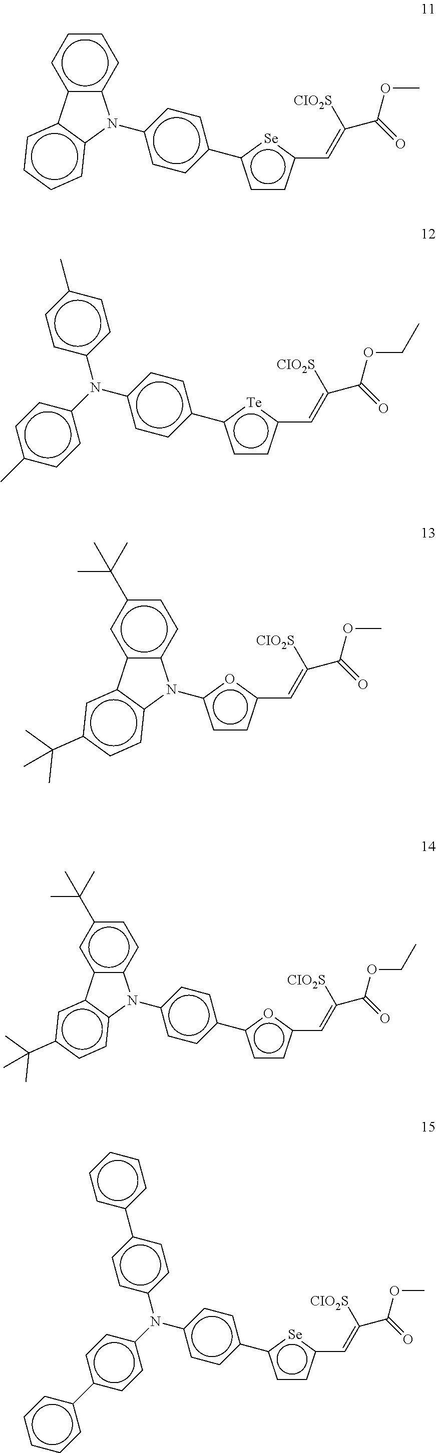

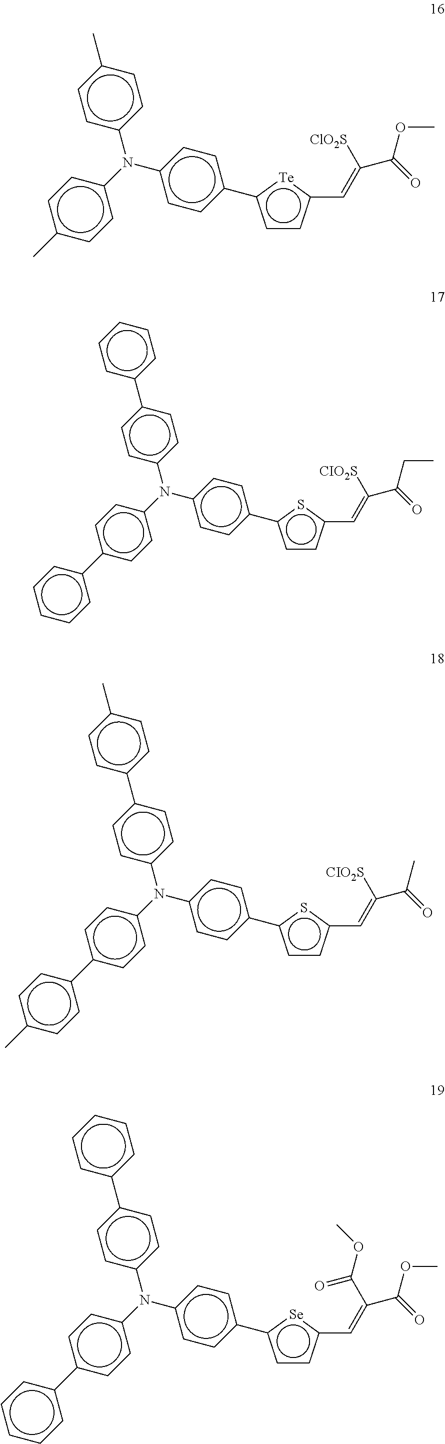

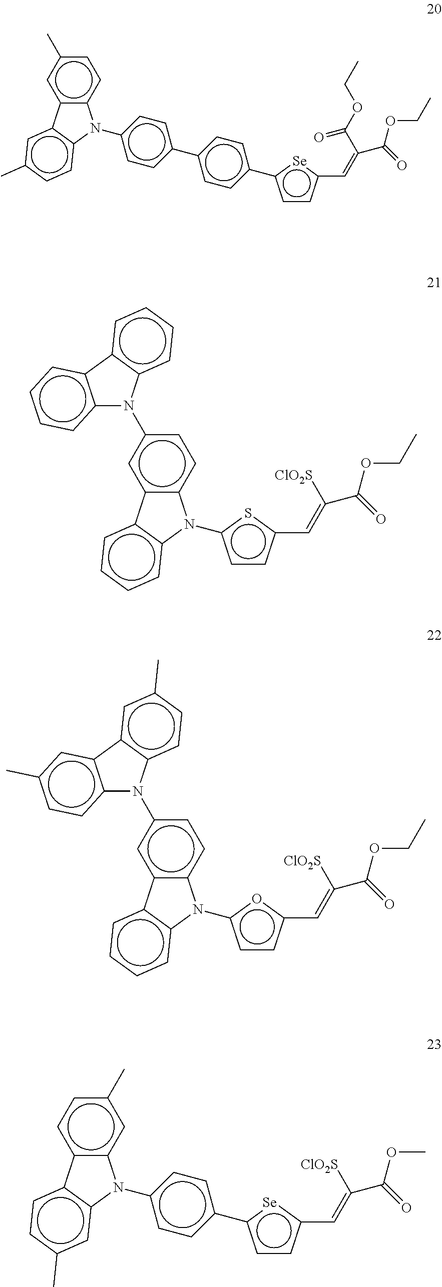

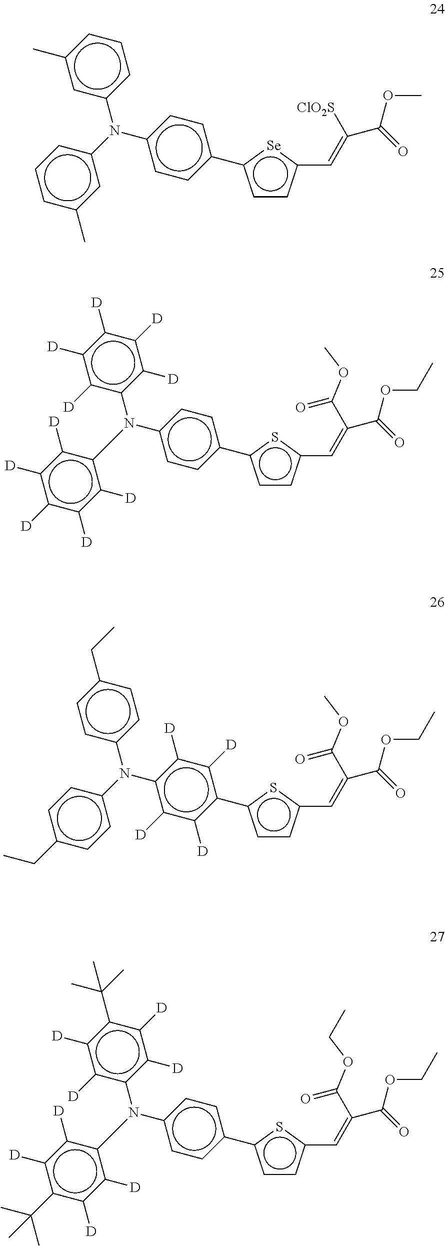

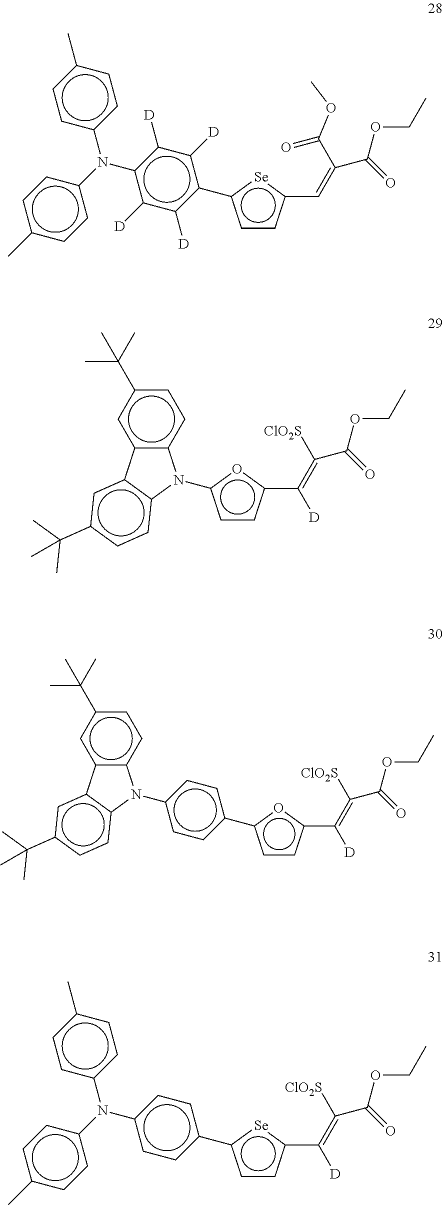

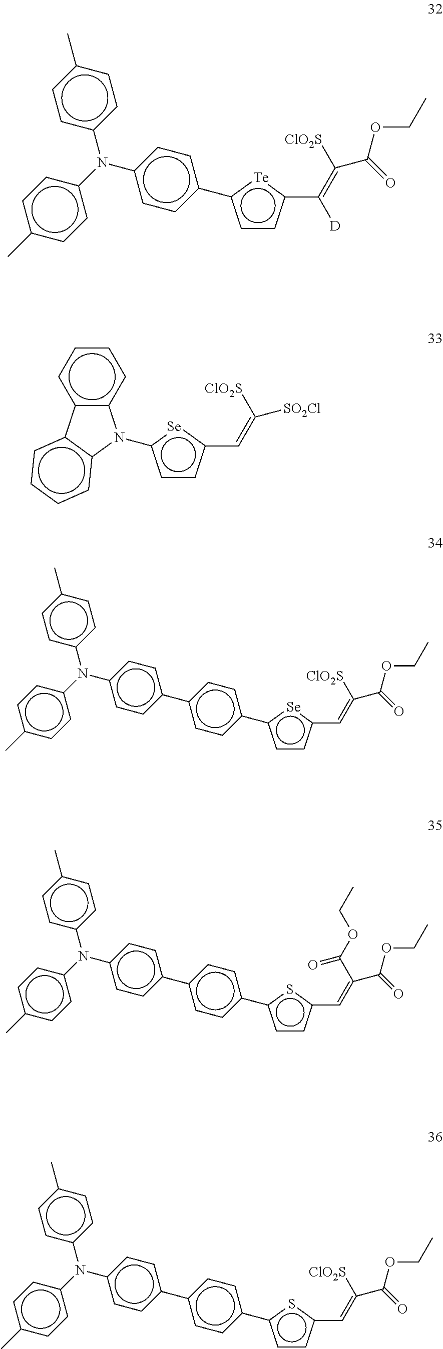

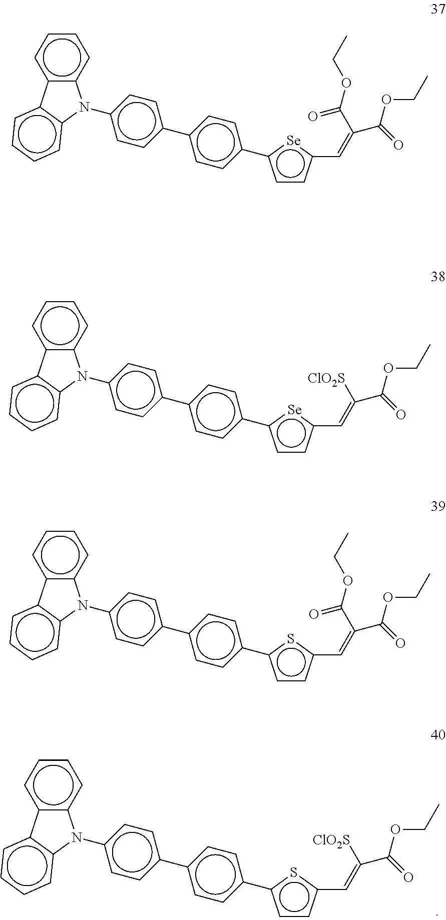

The organic compound represented by Formula 1 may include Z₁ and Z₂ that may each independently be selected from: hydrogen; deuterium; —F; —Cl; —Br; —I; —CF₃; —C(═O)OR₃; —SO₃H; and —S(═O)₂R₄, wherein R₃ and R₄ may each independently be: hydrogen; deuterium; —F; —Cl; —Br; —I; —CF₃; or a C₁-C₂₀ alkyl group substituted with deuterium, —F, —Cl, —Br, —I, —CF₃, or any combination thereof. In some embodiments, when one selected from Z₁ and Z₂ is hydrogen and the other one selected from Z₁ and Z₂ that is not hydrogen is —C(═O)OR₃, R³ may not be hydrogen. For example, i) when Z₁ is hydrogen, Z₂ may not be —COOH, and ii) when Z₁ is —COOH, Z₂ may not be hydrogen. As a result, electron donating characteristics of the organic compound may be improved, and thus, the organic compound may have excellent or suitable characteristics in terms of absorbing maximum light in a green central wavelength region (e.g., in a range of about 500 nm to about 540 nm) and separating the absorbed light into electrons and holes. In some embodiments, an opto-electronic device including the organic compound and the electron accepting compound may have excellent or suitable photoelectric characteristics.

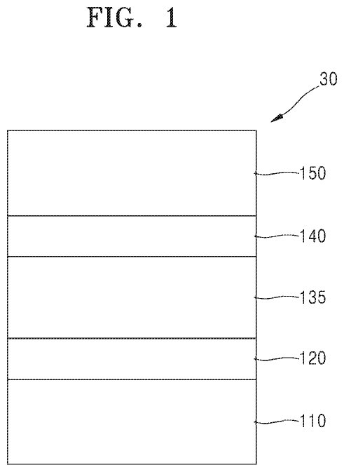

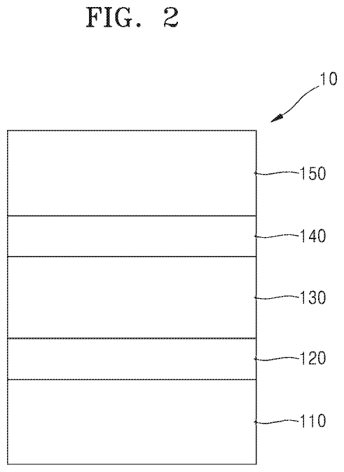

### Description of FIGS. 1 and 2

FIG. 1 is a schematic view of an opto-electronic device 30 according to one or more embodiments. The opto-electronic device 30 may include a first electrode 110, a hole transport region 120, a photoactive layer 135, an electron transport region 140, and a second electrode 150.

FIG. 2 is a schematic view of a light-emitting device 10. The light-emitting device 10 may include a first electrode 110, a hole transport region 120, an emission layer 130, an electron transport region 140, and a second electrode 150.

In one or more embodiments, the first electrode 110, the hole transport region 120, the electron transport region 140, and the second electrode 150 of the opto-electronic device 30 may have a substantially single body (e.g., may be integral) with the first electrode 110, the hole transport region 120, the electron transport region 140, and the second electrode 150 of the light-emitting device 10, respectively. In one or more embodiments, the first electrode 110, the hole transport region 120, the electron transport region 140, and the second electrode 150 of the opto-electronic device 30 may be apart from the first electrode 110, the hole transport region 120, the electron transport region 140, and the second electrode 150 of the light-emitting device 10, respectively, but may include substantially the same material and be formed substantially at the same time as the first electrode 110, the hole transport region 120, the electron transport region 140, and the second electrode 150 of the light-emitting device 10, respectively.

Hereinafter, the structures of the opto-electronic device 30 and the light-emitting device 10 according to embodiments and methods of manufacturing the opto-electronic device 30 and the light-emitting device 10 will be described with reference to FIGS. 1 and 2.

### First Electrode 110

In FIG. 1, a substrate may be additionally arranged under the first electrode 110 or on the second electrode 150. As the substrate, a glass substrate and/or a plastic substrate may be utilized. In one or more embodiments, the substrate may be a flexible substrate, and may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure including (e.g., consisting of) a single layer or a multi-layered structure including a plurality of layers. For example, the first electrode 110 may have a three-layered structure of ITO/Ag/ITO.

### Hole Transport Region 120

The hole transport region 120 may have i) a single-layered structure including (e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The hole transport region 120 may include a hole injection layer, a hole transport layer, an emission auxiliary layer, an electron blocking layer, or any combination thereof.

For example, the hole transport region 120 may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, the layers of each structure being stacked sequentially from the first electrode 110.

The hole transport region may include a compound represented by Formula 201, a compound represented by Formula 202, or any combination thereof:

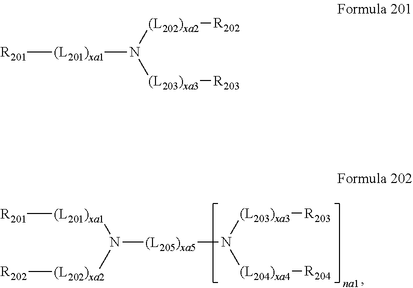

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, —S—*, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (e.g., a carbazole group, etc.) unsubstituted or substituted with at least one R₁₀ₐ (e.g., Compound HT16, etc.),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and
na1 may be an integer from 1 to 4.

For example, each of Formulae 201 and 202 may include at least one of groups represented by Formulae CY201 to CY217:

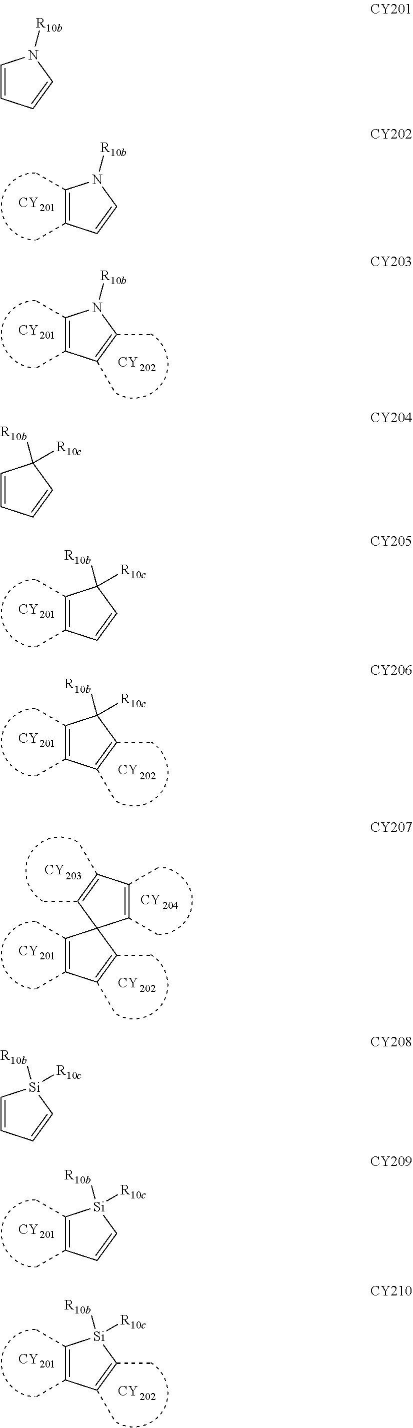

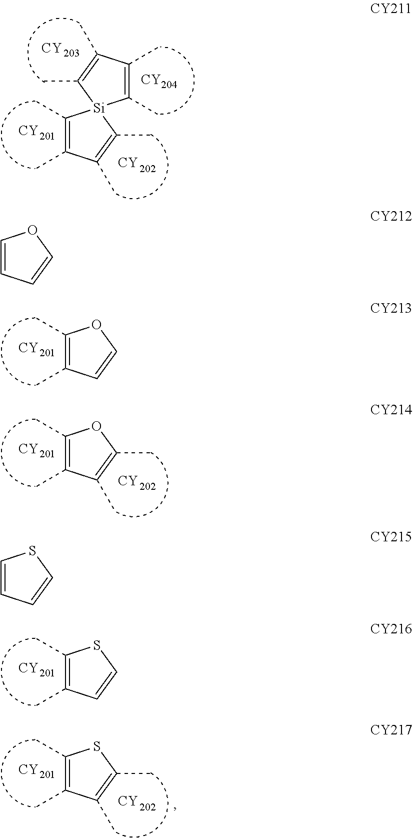

wherein, in Formulae CY201 to CY217, R_{10b} and R_{10c} may each be as defined herein in connection with R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described herein.

In one or more embodiments, ring CY₂₀₁ to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one of groups represented by Formulae CY201 to CY203.

In one or more embodiments, Formula 201 may include at least one of groups represented by Formulae CY201 to CY203 and at least one of groups represented by Formulae CY204 to CY217.

In one or more embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In one or more embodiments, each of Formulae 201 and 202 may not include (e.g., may exclude) a group represented by one of Formulae CY201 to CY203.

In one or more embodiments, each of Formulae 201 and 202 may not include (e.g., may exclude) a group represented by one of Formulae CY201 to CY203, and may include at least one of groups represented by Formulae CY204 to CY217.

In one or more embodiments, each of Formulae 201 and 202 may not include (e.g., may exclude) a group represented by one of Formulae CY201 to CY217.

For example, the hole transport region 120 may include one or more of Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

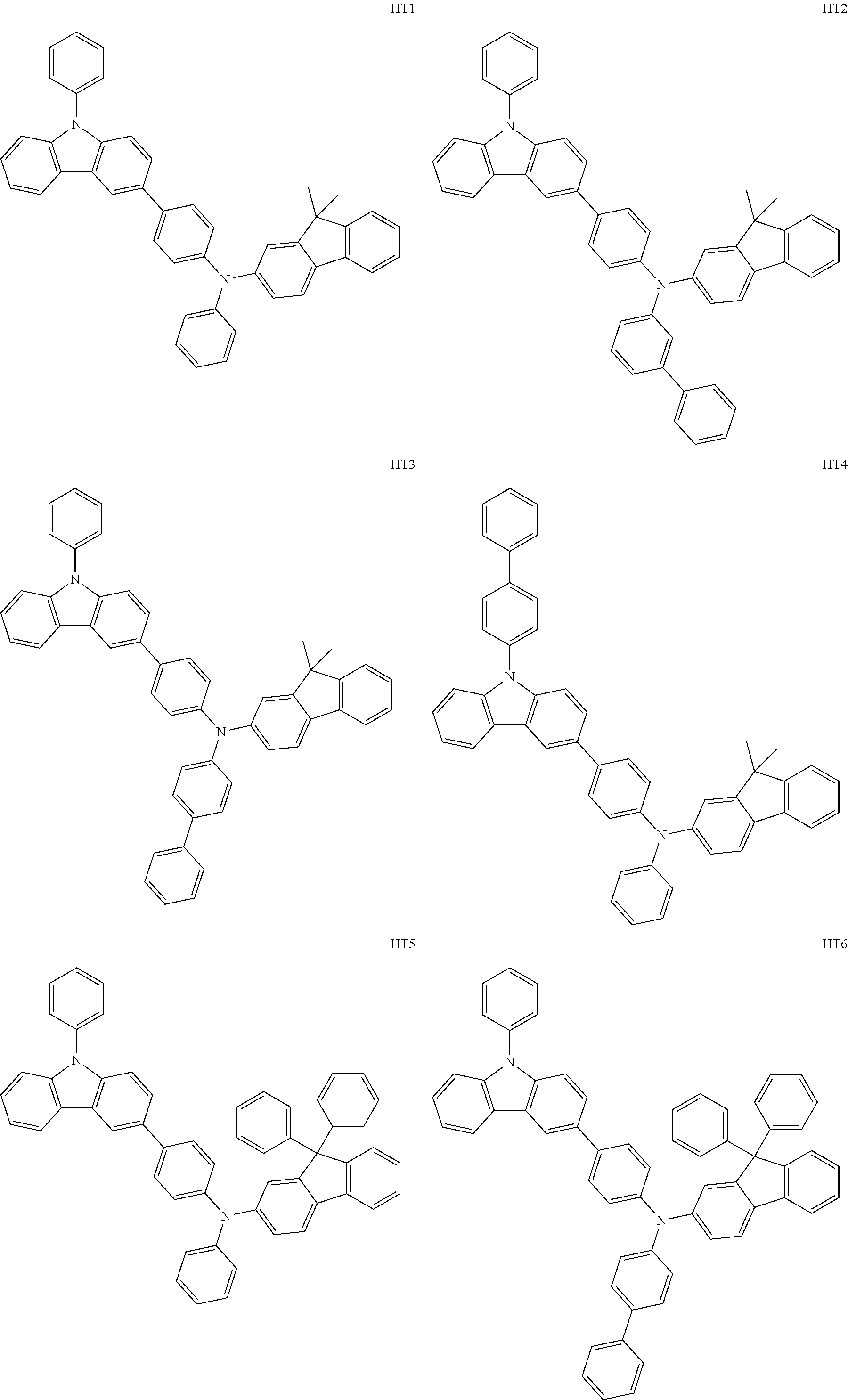

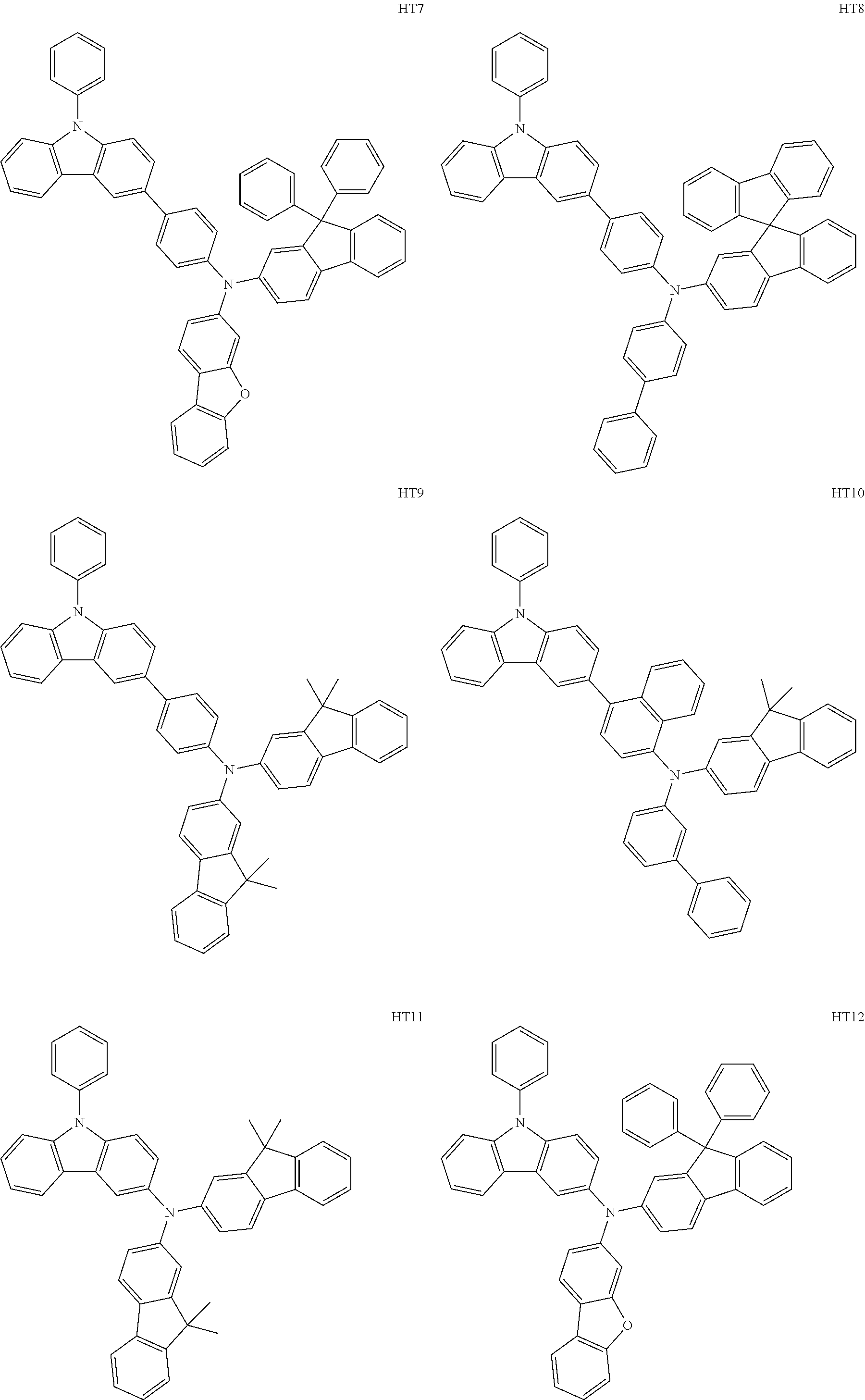

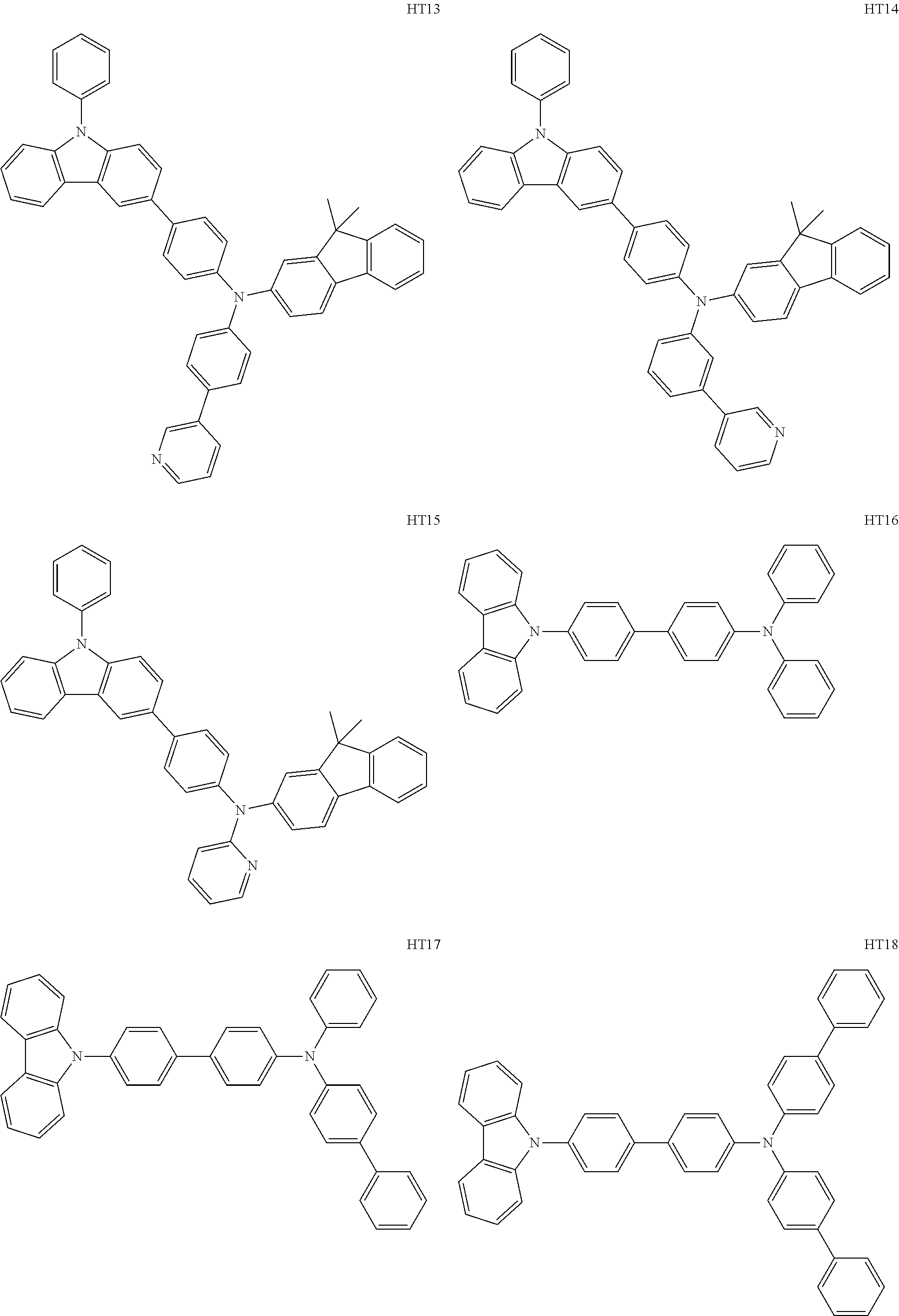

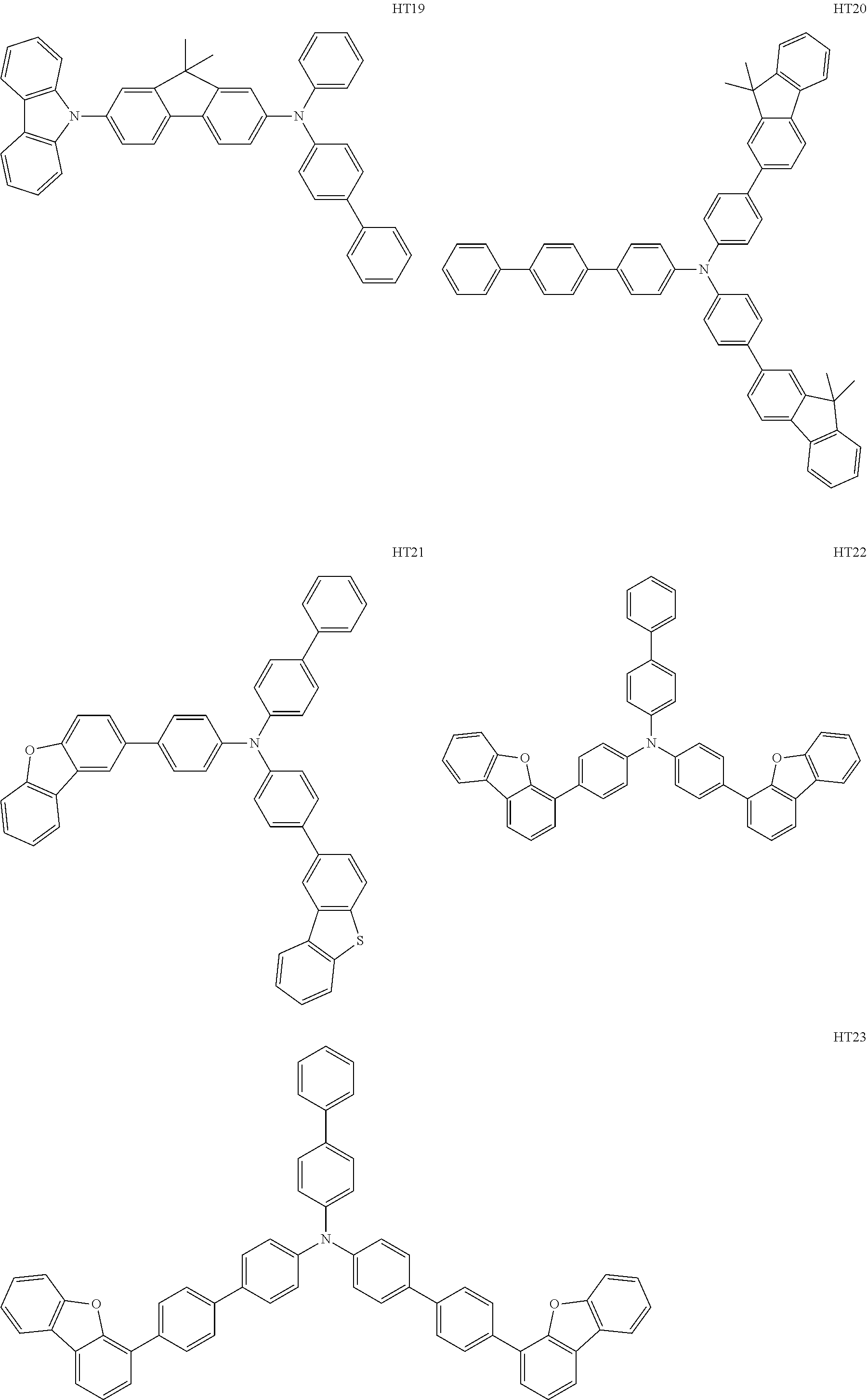

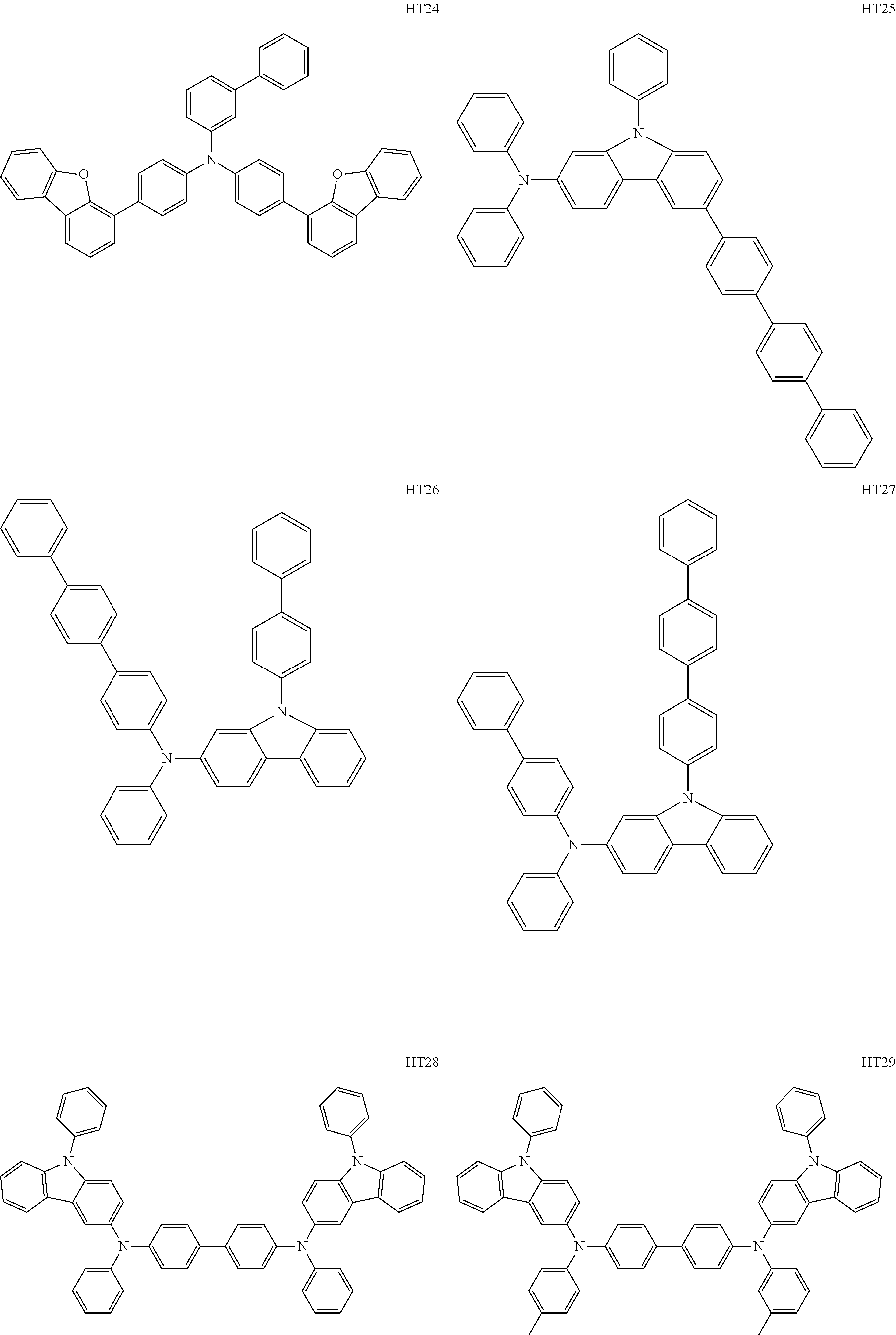

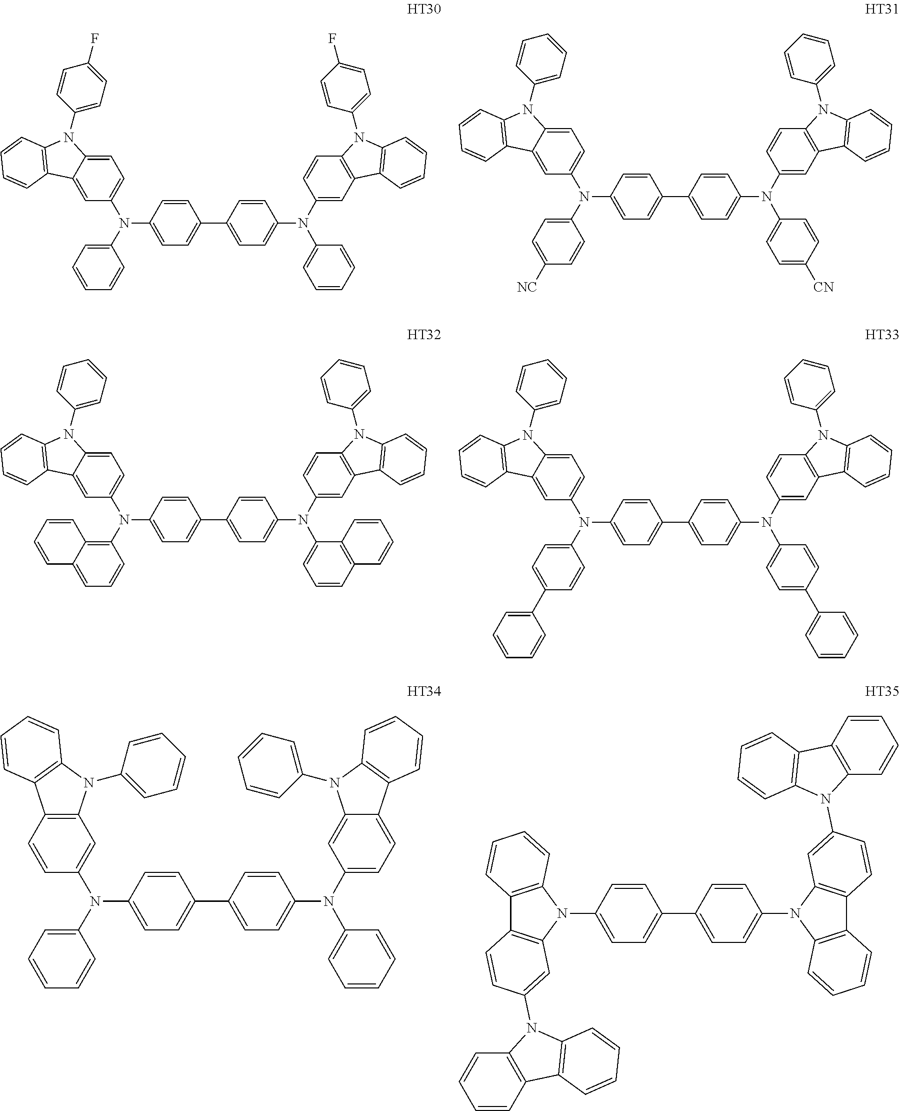

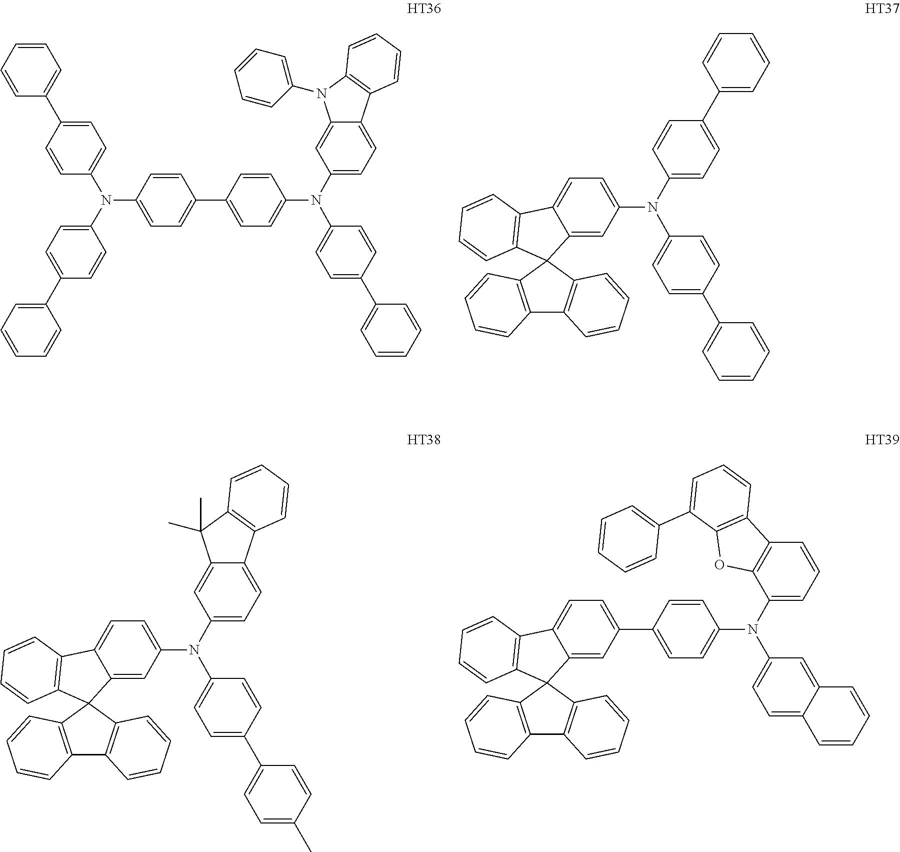 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

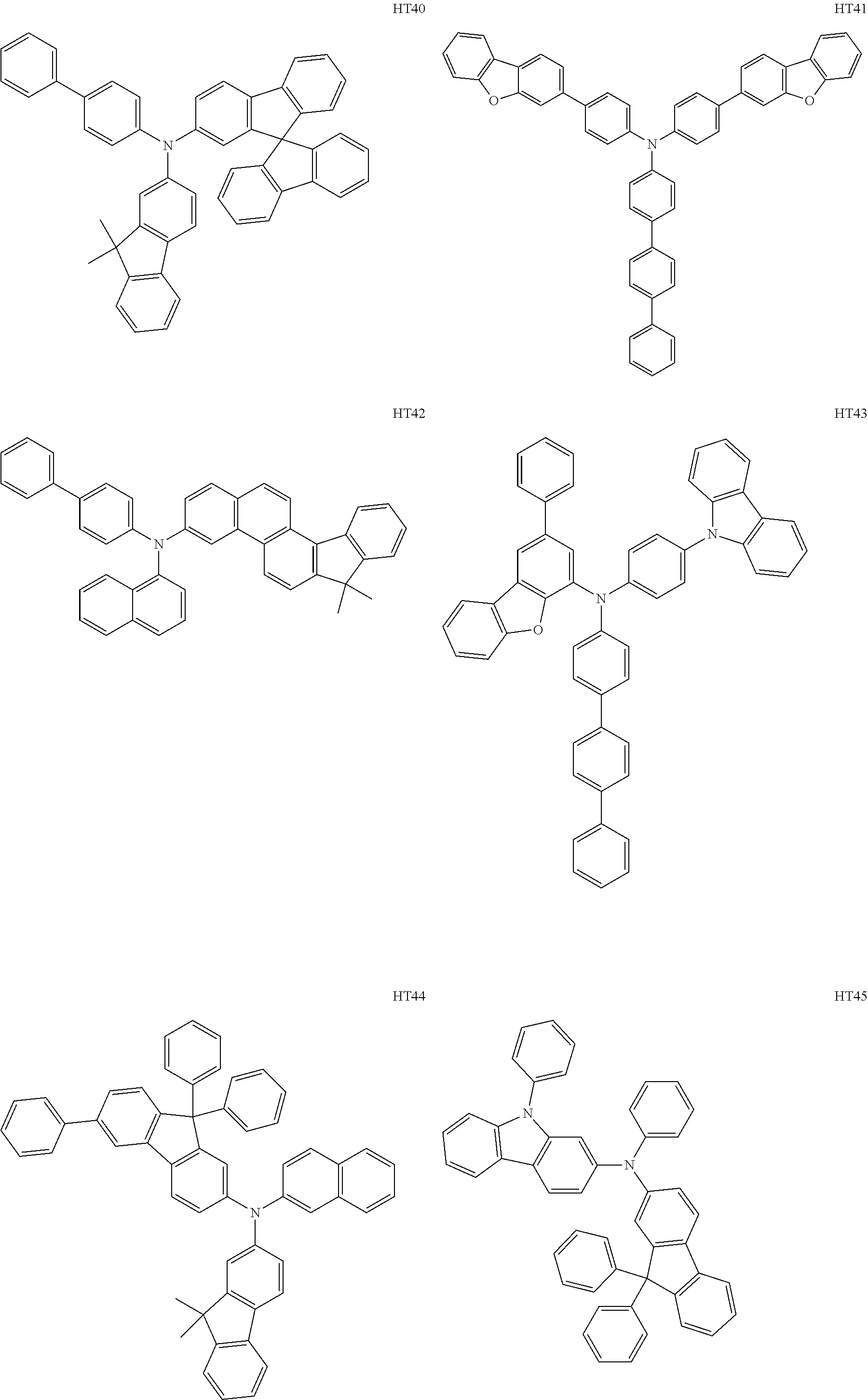

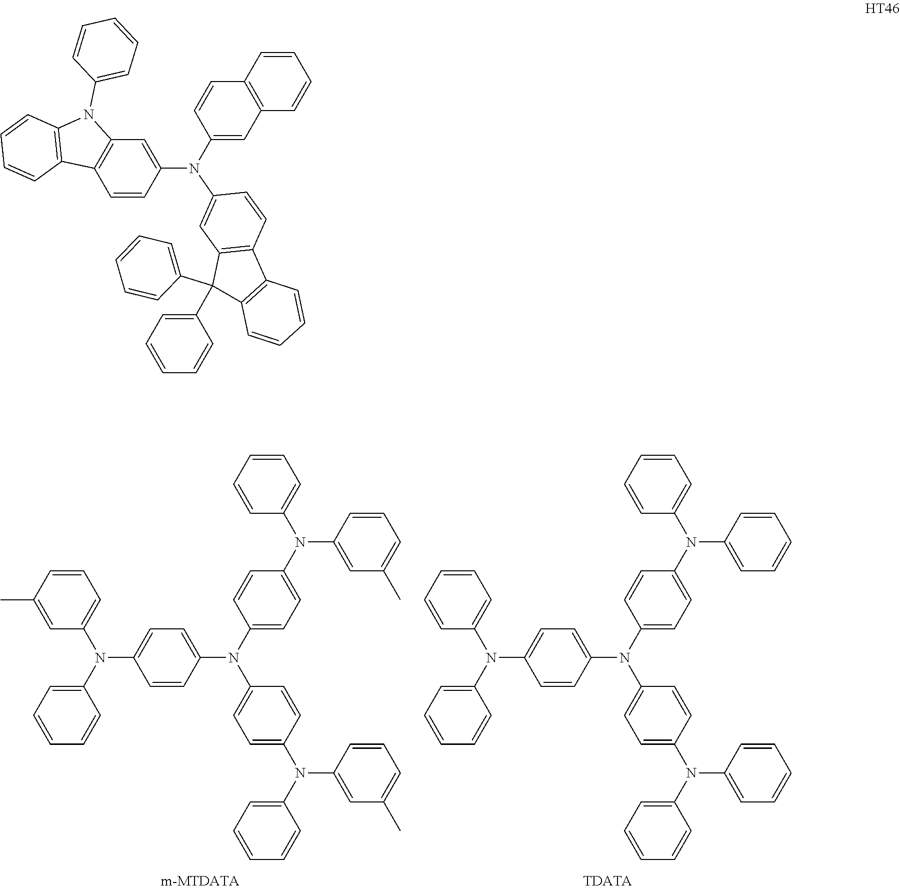

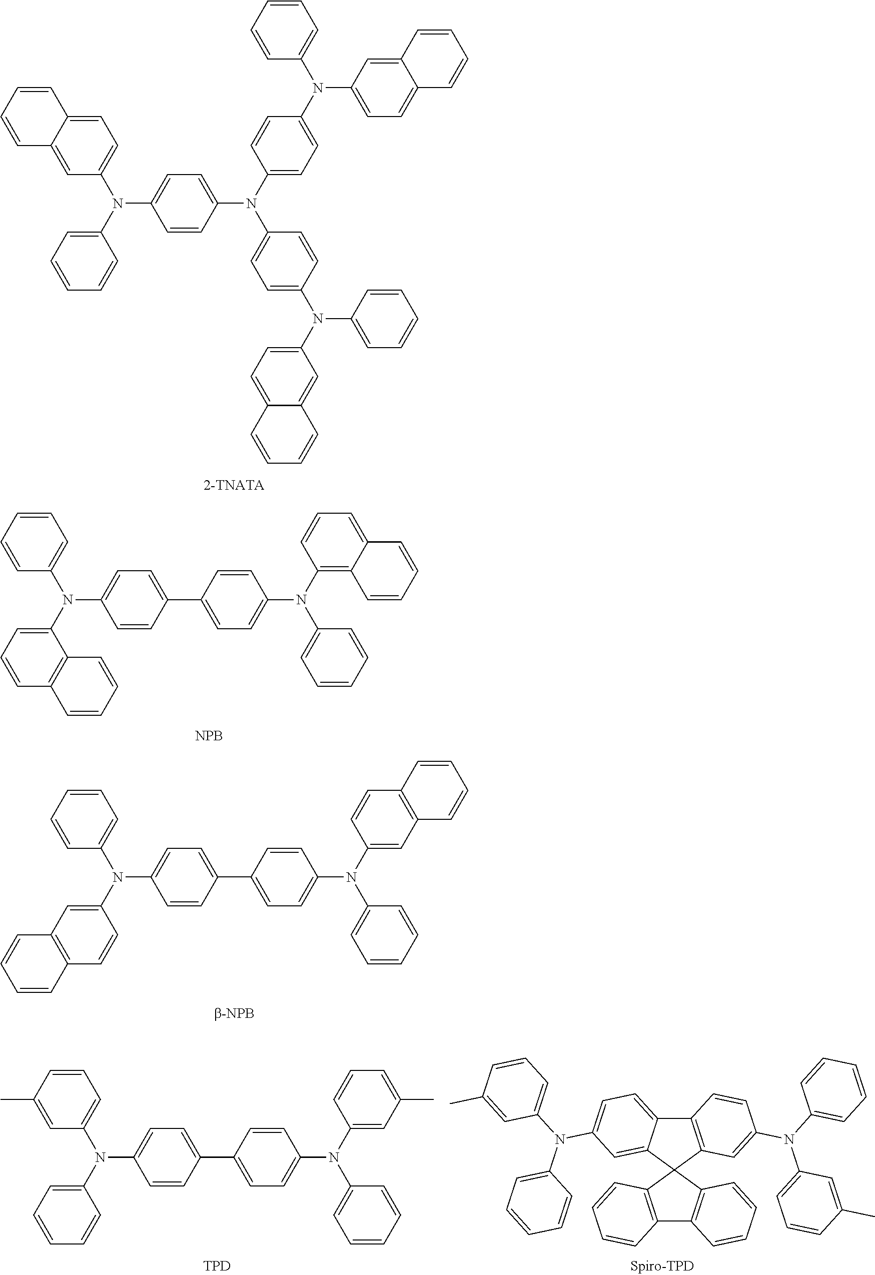

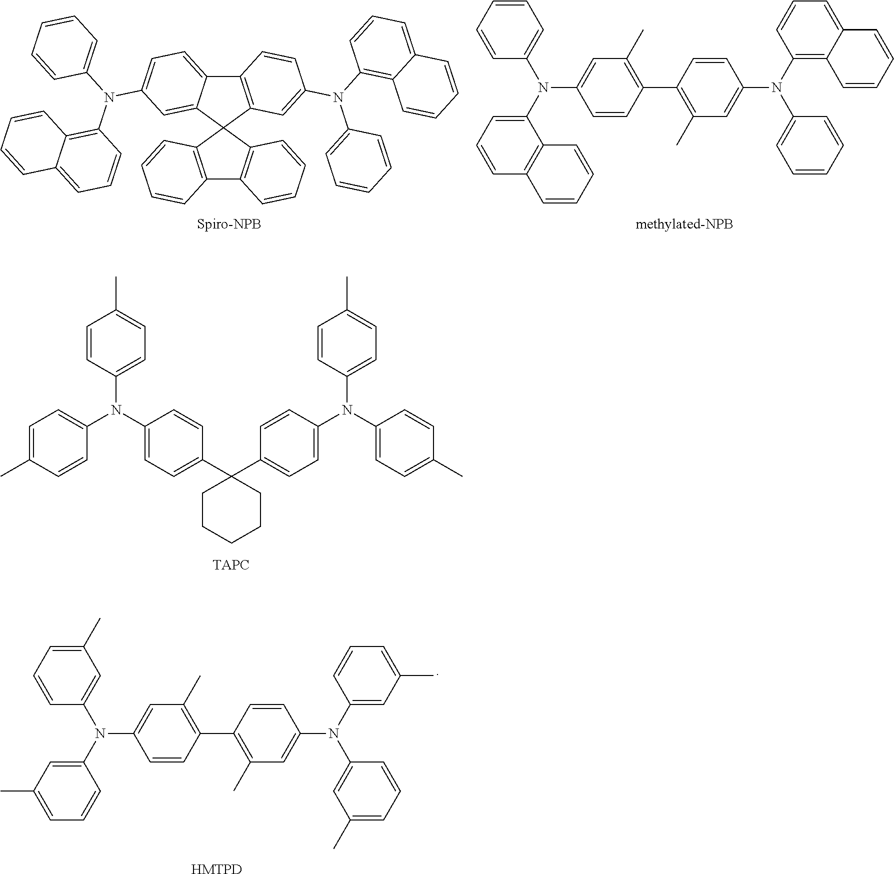

A thickness of the hole transport region 120 may be in a range of about 50 Å to about 10,000 Å, for example, about 100 Å to about 4,000 Å. When the hole transport region 120 includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region 120, the hole injection layer, and the hole transport layer are within any of their respective ranges, satisfactory or suitable hole transporting characteristics may be obtained without a substantial increase in driving voltage.

The emission auxiliary layer may increase light-emission efficiency by compensating for an optical resonance distance according to the wavelength of light emitted by the emission layer 130, and the electron blocking layer may block or reduce the leakage of electrons from the emission layer 130 to the hole transport region 120. Material(s) that may be included in the hole transport region 120 may be included in the emission auxiliary layer and/or the electron blocking layer.

p-Dopant

The hole transport region 120 may further include, in addition to the materials as described above, a charge-generation material for improving conductive properties. The charge-generation material may be substantially uniformly or substantially non-uniformly dispersed in the hole transport region 120 (e.g., in the form of a single layer including (e.g., consisting of) a charge-generation material).

The charge-generation material may be, for example, a p-dopant.

For example, a LUMO energy level of the p-dopant may be −3.5 eV or less.

In one or more embodiments, the p-dopant may include a quinone derivative, a cyano group-containing compound, a compound containing element EL1 and element EL2, or any combination thereof.

Examples of the quinone derivative may include TCNQ, F4-TCNQ, and the like.

Examples of the cyano group-containing compound may include HAT-CN, a compound represented by Formula 221, and the like:

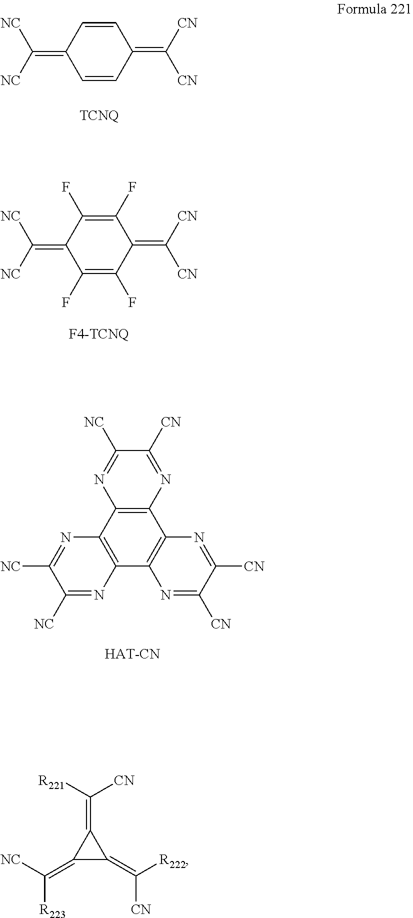

wherein, in Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one of R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound containing element EL1 and element EL2, element EL1 may be a metal, a metalloid, or any combination thereof, and element EL2 may be a non-metal, a metalloid, or any combination thereof.

Examples of the metal may include: an alkali metal (e.g., lithium (Li), sodium (Na), potassium (K), rubidium (Rb), cesium (Cs), etc.); an alkaline earth metal (e.g., beryllium (Be), magnesium (Mg), calcium (Ca), strontium (Sr), barium (Ba), etc.); a transition metal (e.g., titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (e.g., zinc (Zn), indium (In), tin (Sn), etc.); a lanthanide metal (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.); and the like.

Examples of the metalloid may include silicon (Si), antimony (Sb), tellurium (Te), and/or the like.

Examples of the non-metal may include oxygen (O), halogen (e.g., F, Cl, Br, I, etc.), and/or the like.

Examples of the compound containing element EL1 and element EL2 may include a metal oxide, a metal halide (e.g., a metal fluoride, a metal chloride, a metal bromide, a metal iodide, etc.), a metalloid halide (e.g., a metalloid fluoride, a metalloid chloride, a metalloid bromide, a metalloid iodide, etc.), a metal telluride, and any combination thereof.

Examples of the metal oxide may include a tungsten oxide (e.g., WO, W₂O₃, WO₂, WO₃, W₂O₅, etc.), a vanadium oxide (e.g., VO, V₂O₃, VO₂, V₂O₅, etc.), a molybdenum oxide (e.g., MoO, Mo₂O₃, MoO₂, MoO₃, Mo₂O₅, etc.), a rhenium oxide (e.g., ReO₃, etc.), and/or the like.

Examples of the metal halide may include an alkali metal halide, an alkaline earth metal halide, a transition metal halide, a post-transition metal halide, a lanthanide metal halide, and/or the like.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, CsI, and/or the like.

Examples of the alkaline earth metal halide may include BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, BeI₂, MgI₂, CaI₂, SrI₂, BaI₂, and/or the like.

Examples of the transition metal halide may include a titanium halide (e.g., TiF₄, TiCl₄, TiBr₄, TiI₄, etc.), a zirconium halide (e.g., ZrF₄, ZrCl₄, ZrBr₄, ZrI₄, etc.), a hafnium halide (e.g., HfF₄, HfCl₄, HfBr₄, HfI₄, etc.), a vanadium halide (e.g., VF₃, VCl₃, VBr₃, VI₃, etc.), a niobium halide (e.g., NbF₃, NbCl₃, NbBr₃, NbI₃, etc.), a tantalum halide (e.g., TaF₃, TaCl₃, TaBr₃, TaI₃, etc.), a chromium halide (e.g., CrF₃, CrCl₃, CrBr₃, CrI₃, etc.), a molybdenum halide (e.g., MoF₃, MoCl₃, MoBr₃, MoI₃, etc.), a tungsten halide (e.g., WF₃, WCl₃, WBr₃, WI₃, etc.), a manganese halide (e.g., MnF₂, MnCl₂, MnBr₂, MnI₂, etc.), a technetium halide (e.g., TcF₂, TcCl₂, TcBr₂, TcI₂, etc.), a rhenium halide (e.g., ReF₂, ReCl₂, ReBr₂, ReI₂, etc.), an iron halide (e.g., FeF₂, FeCl₂, FeBr₂, FeI₂, etc.), a ruthenium halide (e.g., RuF₂, RuCl₂, RuBr₂, RuI₂, etc.), an osmium halide (e.g., OsF₂, OsCl₂, OsBr₂, OsI₂, etc.), a cobalt halide (e.g., CoF₂, COCl₂, CoBr₂, CoI₂, etc.), a rhodium halide (e.g., RhF₂, RhCl₂, RhBr₂, RhI₂, etc.), an iridium halide (e.g., IrF₂, IrCl₂, IrBr₂, IrI₂, etc.), a nickel halide (e.g., NiF₂, NiCl₂, NiBr₂, NiI₂, etc.), a palladium halide (e.g., PdF₂, PdCl₂, PdBr₂, PdI₂, etc.), a platinum halide (e.g., PtF₂, PtCl₂, PtBr₂, PtI₂, etc.), a copper halide (e.g., CuF, CuCl, CuBr, CuI, etc.), a silver halide (e.g., AgF, AgCl, AgBr, AgI, etc.), a gold halide (e.g., AuF, AuCl, AuBr, AuI, etc.), and/or the like.

Examples of the post-transition metal halide may include a zinc halide (e.g., ZnF₂, ZnCl₂, ZnBr₂, ZnI₂, etc.), an indium halide (e.g., InI₃, etc.), a tin halide (e.g., SnI₂, etc.), and/or the like.

Examples of the lanthanide metal halide may include YbF, YbF₂, YbF₃, SmF₃, YbCl, YbCl₂, YbCl₃ SmCl₃, YbBr, YbBr₂, YbBr₃, SmBr₃, YbI, YbI₂, YbI₃, SmI₃, and/or the like.

Examples of the metalloid halide may include an antimony halide (e.g., SbCl₅, etc.) and/or the like.

Examples of the metal telluride may include an alkali metal telluride (e.g., Li₂Te, Na₂Te, K₂Te, Rb₂Te, Cs₂Te, etc.), an alkaline earth metal telluride (e.g., BeTe, MgTe, CaTe, SrTe, BaTe, etc.), a transition metal telluride (e.g., TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), a post-transition metal telluride (e.g., ZnTe, etc.), a lanthanide metal telluride (e.g., LaTe, CeTe, PrTe, NdTe, PmTe, EuTe, GdTe, TbTe, DyTe, HoTe, ErTe, TmTe, YbTe, LuTe, etc.), and/or the like.

### Emission Layer 130

The light-emitting device 10 may include the emission layer 130 arranged on the hole transport region 120.

The emission layer 130 may further include, in addition to one or more suitable organic materials, a metal-containing compound, such as an organometallic compound, an inorganic material, such as a quantum dot, and/or the like.

In one or more embodiments, the emission layer 130 may include i) two or more emitting units sequentially stacked between the first electrode 110 and the second electrode 150, and ii) a charge generation layer arranged between the two or more emitting units. When the emission layer 130 includes the emitting units and the charge generation layer as described above, the light-emitting device 10 may be a tandem light-emitting device.

When the light-emitting device 10 is a full-color light-emitting device, the emission layer 130 may be patterned into a red emission layer, a green emission layer, and/or a blue emission layer, according to a subpixel. In one or more embodiments, the emission layer 130 may have a stacked structure of two or more layers of a red emission layer, a green emission layer, and a blue emission layer, in which the two or more layers contact each other or are separated from each other to emit white light. In one or more embodiments, the emission layer 130 may include two or more materials of a red light-emitting material, a green light-emitting material, and a blue light-emitting material, in which the two or more materials are mixed with each other in a single layer to emit white light.

The emission layer 130 may include a host and a dopant. The dopant may include a phosphorescent dopant, a fluorescent dopant, or any combination thereof.

An amount of the dopant in the emission layer 130 may be in a range of about 0.01 part by weight to about 15 parts by weight based on 100 parts by weight of the host.

In one or more embodiments, the emission layer 130 may include a quantum dot.

In one or more embodiments, the emission layer 130 may include a delayed fluorescence material. The delayed fluorescence material may serve as a host or a dopant in the emission layer 130.

A thickness of the emission layer 130 may be in a range of about 100 Å to about 1,000 Å, for example, about 200 Å to about 600 Å. When the thickness of the emission layer 130 is within these ranges, excellent or suitable luminescence characteristics may be obtained without a substantial increase in driving voltage.

### Host

The host may include a compound represented by Formula 301:

[Ar₃₀₁]_{xb11}-[(L₃₀₁)_{xb1}-R₃₀₁]_{xb21}, Formula 301
wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ ms may each be as defined herein in connection with Q₁.

For example, when xb11 in Formula 301 is 2 or more, two or more of Ar₃₀₁(s) may be linked to each other via a single bond.

In one or more embodiments, the host may include a compound represented by Formula 301-1, a compound represented by Formula 301-2, or any combination thereof:

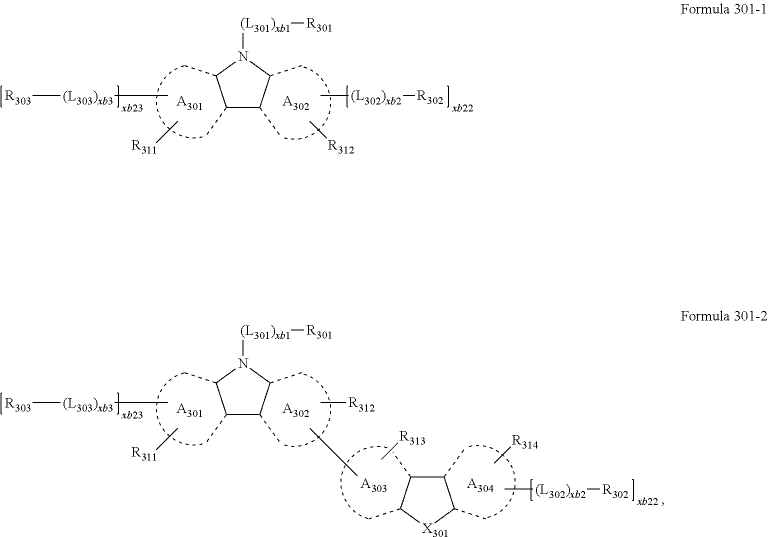

wherein, in Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be as defined herein,
L₃₀₂ to L₃₀₄ may each independently be as defined herein in connection with L₃₀₁,
xb2 to xb4 may each independently be as defined herein in connection with xb1, and
R₃₀₂ to R₃₀₅ and R₃₁₁ to R₃₁₄ may each be as defined herein in connection with R₃₀₁.

In one or more embodiments, the host may include an alkali earth metal complex, a post-transition metal complex, or any combination thereof. For example, the host may include a Be complex (e.g., Compound H55), a Mg complex, a Zn complex, or any combination thereof.

In one or more embodiments, the host may include one or more of Compounds H1 to H128, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di(carbazol-9-yl)benzene (mCP), 1,3,5-tri(carbazol-9-yl)benzene (TCP), or any combination thereof:

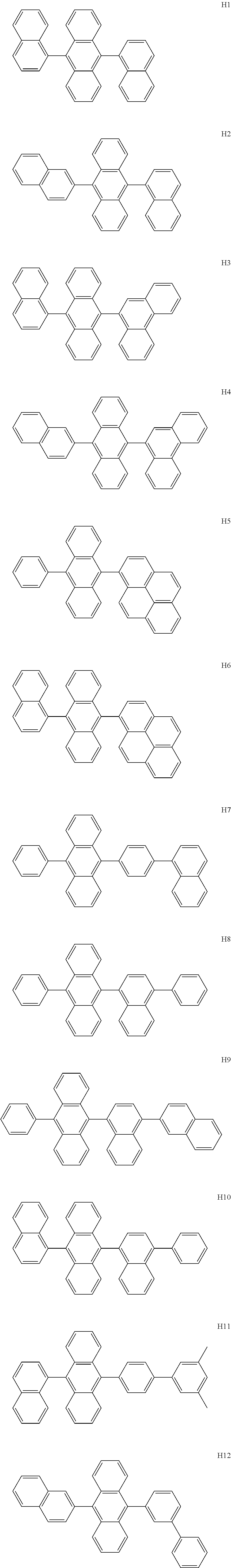

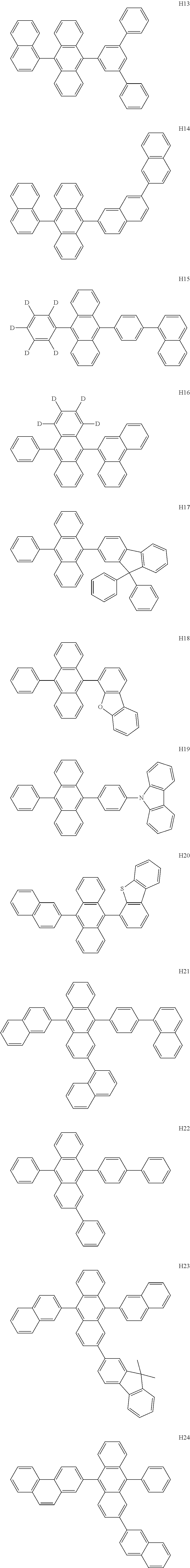

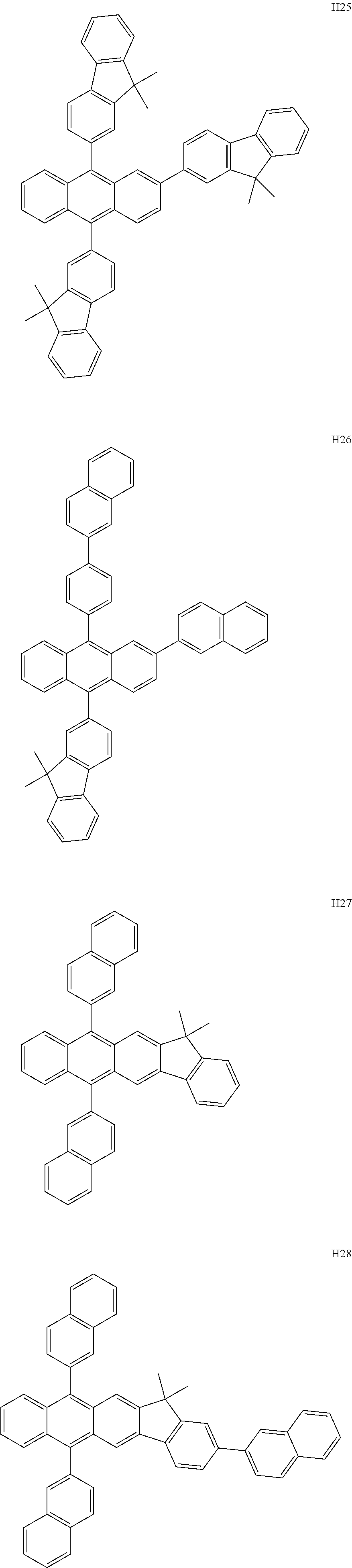

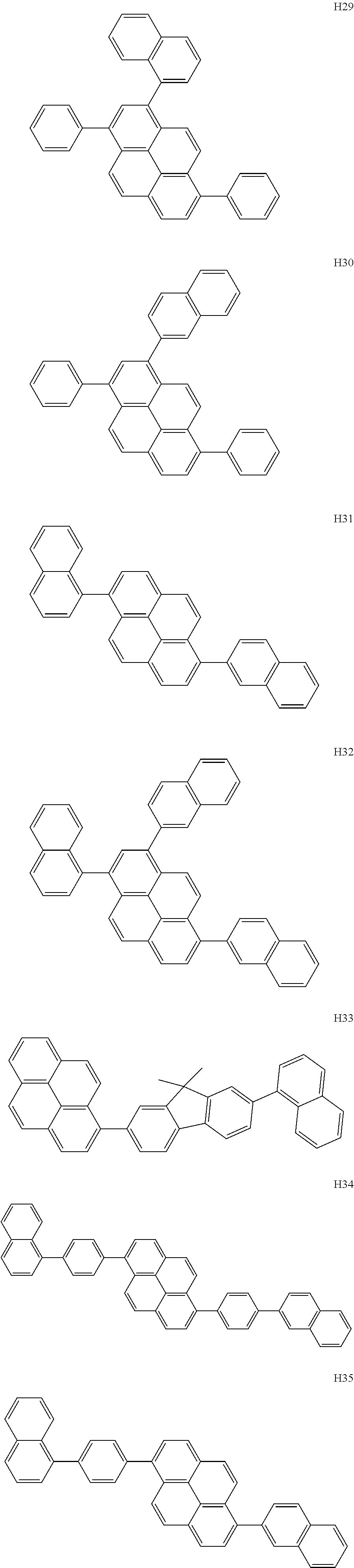

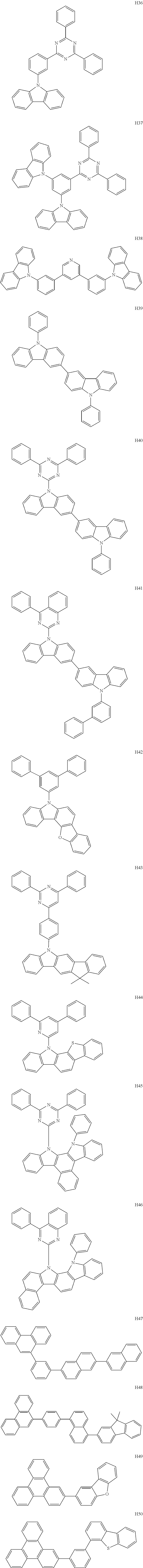

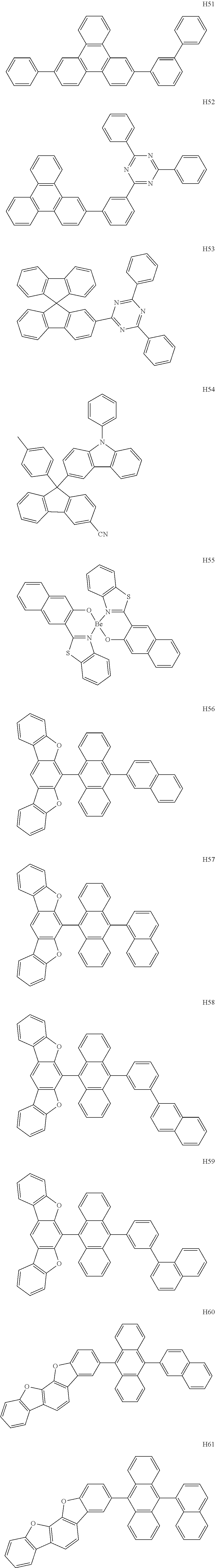

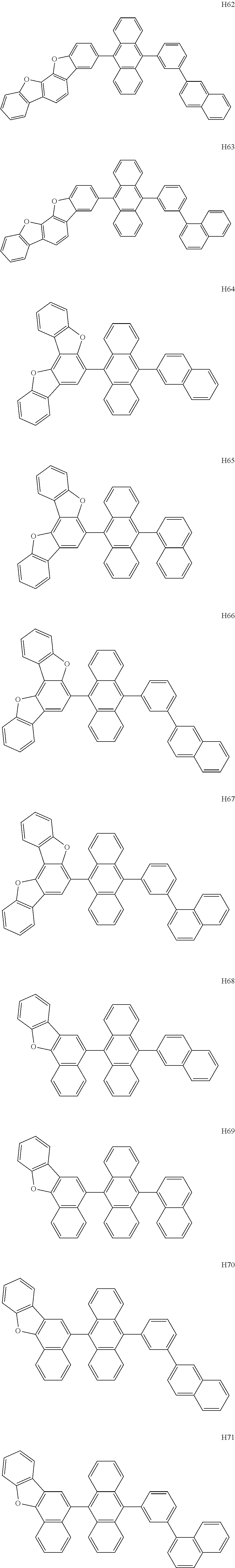

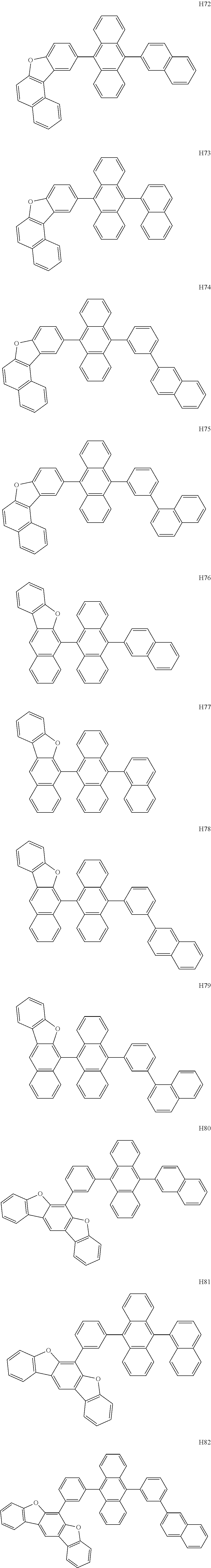

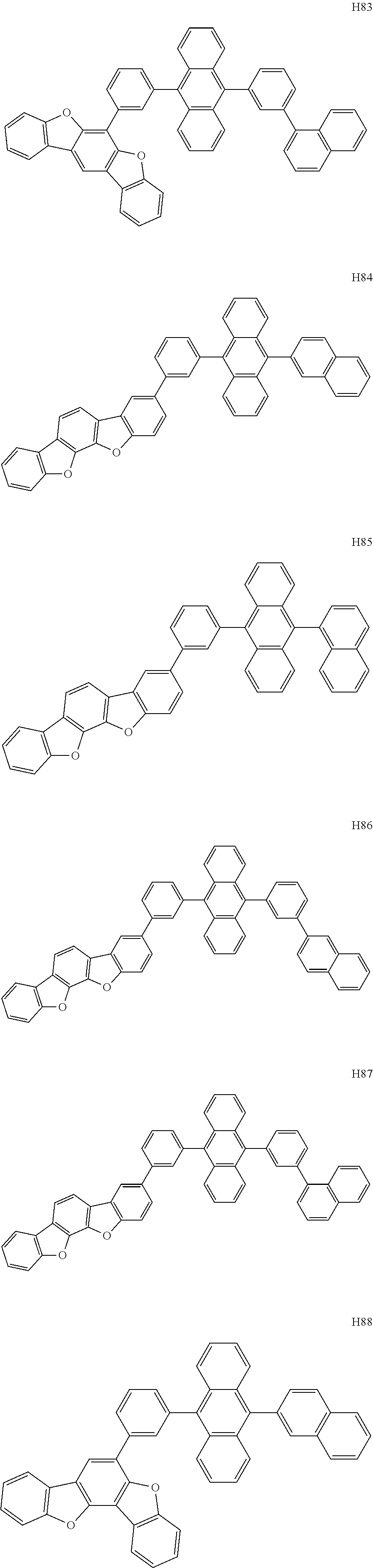

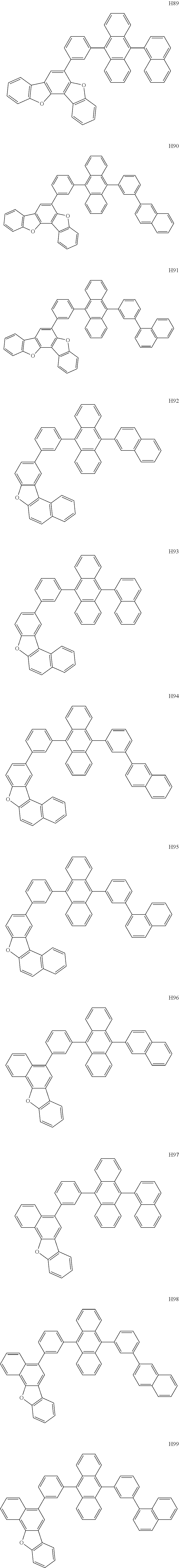

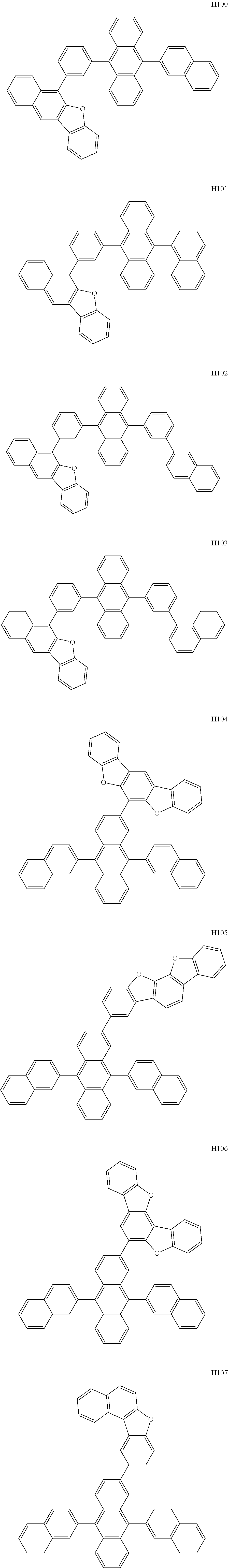

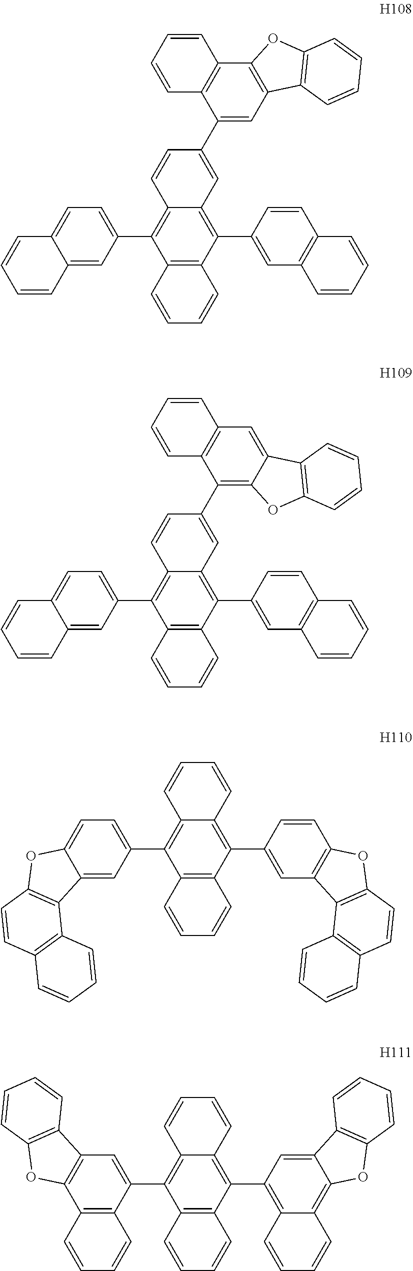 [SMILES: c1ccc2cc(-c3c4ccccc4c(-c4ccc5ccccc5c4)c4cc(-c5cc6c7ccccc7oc6c6ccccc56)ccc34)ccc2c1 ; c1ccc2cc(-c3c4ccccc4c(-c4ccc5ccccc5c4)c4cc(-c5c6ccccc6cc6c5oc5ccccc56)ccc34)ccc2c1 ; c1ccc2c(c1)ccc1oc3ccc(-c4c5ccccc5c(-c5ccc6oc7ccc8ccccc8c7c6c5)c5ccccc45)cc3c12 ; c1ccc2c(c1)oc1c3ccccc3c(-c3c4ccccc4c(-c4cc5c6ccccc6oc5c5ccccc45)c4ccccc34)cc21]

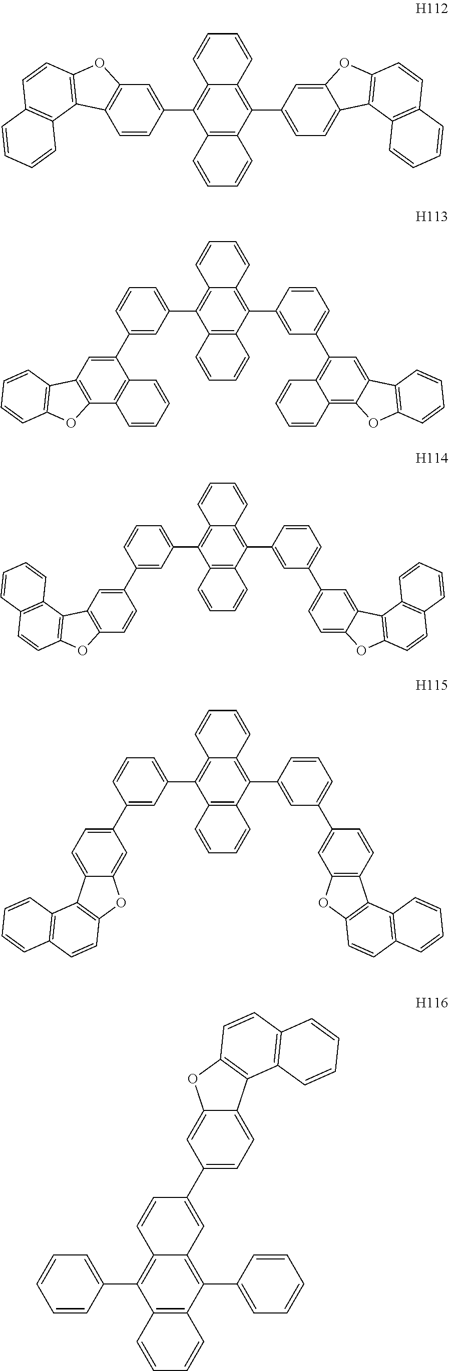 [SMILES: c1ccc2c(c1)ccc1oc3cc(-c4c5ccccc5c(-c5ccc6c(c5)oc5ccc7ccccc7c56)c5ccccc45)ccc3c12 ; c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1]

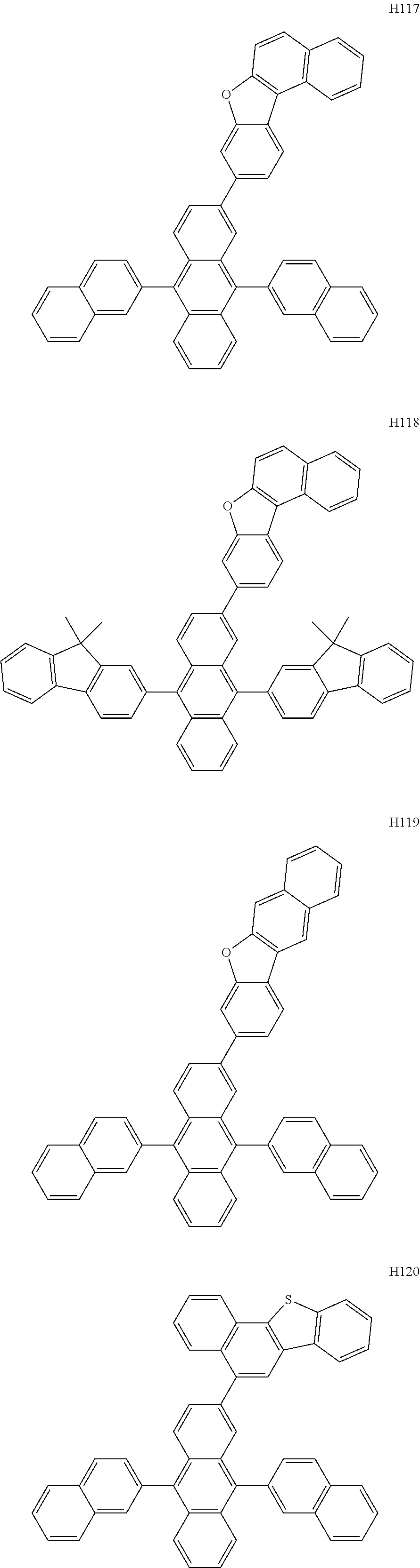 [SMILES: c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21 ; c1ccc2cc(-c3c4ccccc4c(-c4ccc5ccccc5c4)c4cc(-c5ccc6c(c5)oc5cc7ccccc7cc56)ccc34)ccc2c1 ; c1ccc2cc(-c3c4ccccc4c(-c4ccc5ccccc5c4)c4cc(-c5cc6c7ccccc7sc6c6ccccc56)ccc34)ccc2c1]

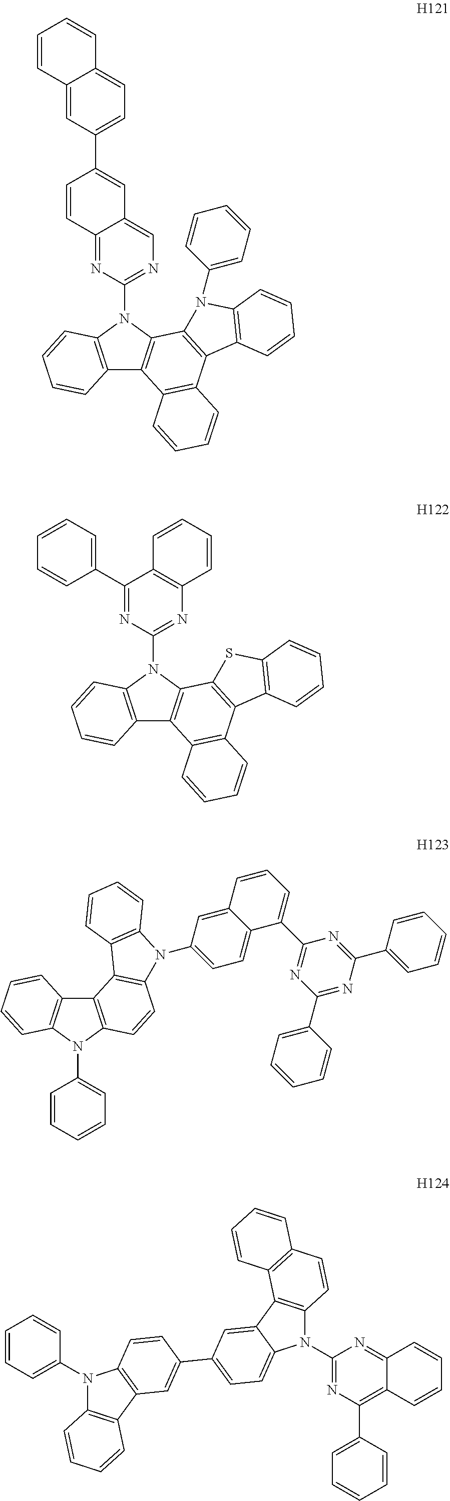 [SMILES: c1ccc(-n2c3ccccc3c3c4ccccc4c4c5ccccc5n(-c5ncc6cc(-c7ccc8ccccc8c7)ccc6n5)c4c32)cc1 ; c1ccc(-c2nc(-n3c4ccccc4c4c5ccccc5c5c6ccccc6sc5c43)nc3ccccc23)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

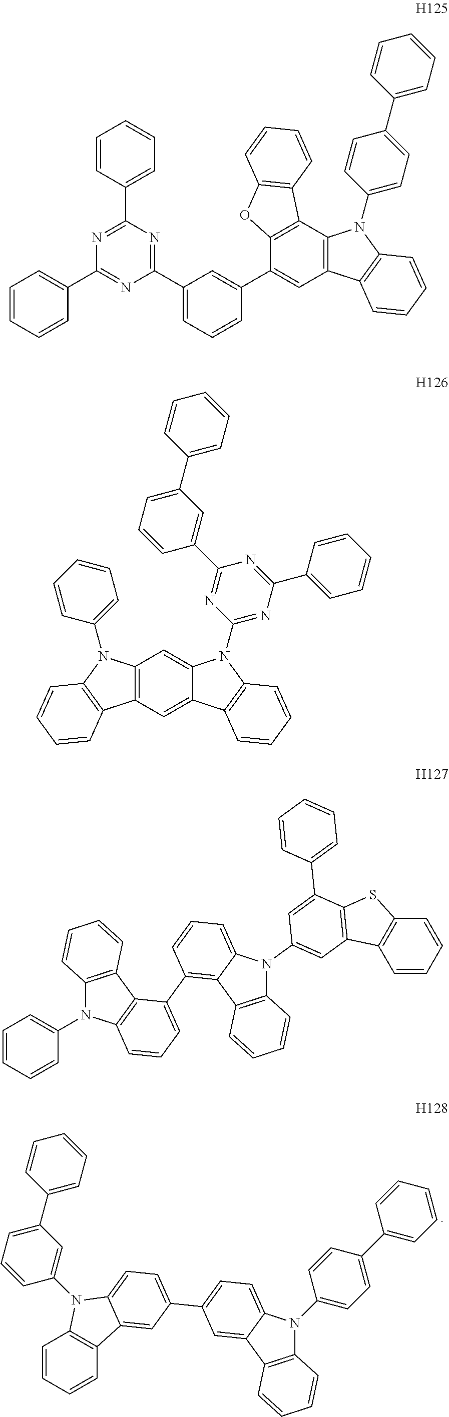 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

### Phosphorescent Dopant

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

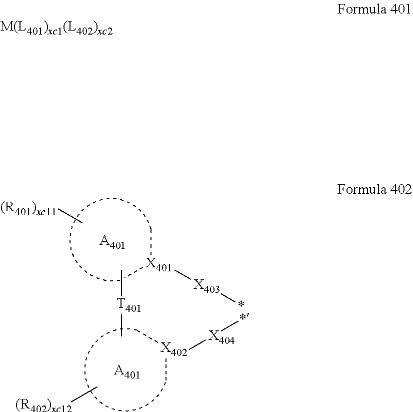

wherein, in Formulae 401 and 402,
M may be a transition metal (e.g., iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein, when xc1 is 2 or more, two or more of L₄₀₁(s) may be identical to or different from each other,
L₄₀₂ may be an organic ligand, and xc2 may be 0, 1, 2, 3, or 4, wherein, when xc2 is 2 or more, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)=C(Q₄₁₂)-*′, *—C(Q₄₁₁)=*′, or *═C═*′,
X₄₀₃ and X₄₀₄ may each independently be a chemical bond (e.g., a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each be as defined herein in connection with Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each be as defined herein in connection with Q₁,
xc11 and xc12 may each independently be an integer from 0 to 10, and
* and *′ in Formula 402 may each indicate a binding site to M in Formula 401.

For example, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 401 is 2 or more, two ring A₄₀₁(s) in two or more of L₄₀₁(s) may optionally be linked to each other via T₄₀₂, which is a linking group, and/or two ring A₄₀₂(s) may optionally be linked to each other via T₄₀₃, which is a linking group (see e.g., Compounds PD1 to PD4 and PD7). T₄₀₂ and T₄₀₃ may each be as defined herein in connection with T₄₀₁.

L₄₀₂ in Formula 401 may be an organic ligand. For example, L₄₀₂ may include a halogen group, a diketone group (e.g., an acetylacetonate group), a carboxylic acid group (e.g., a picolinate group), —C(═O), an isonitrile group, a —CN group, a phosphorus group (e.g., a phosphine group, a phosphite group, etc.), or any combination thereof.

The phosphorescent dopant may include, for example, one of Compounds PD1 to PD39 or any combination thereof:

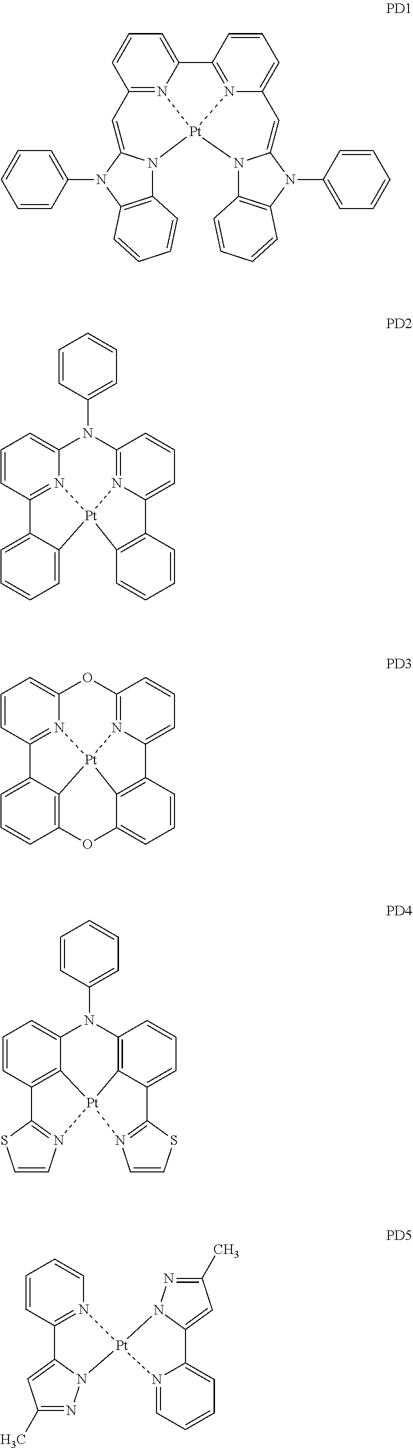

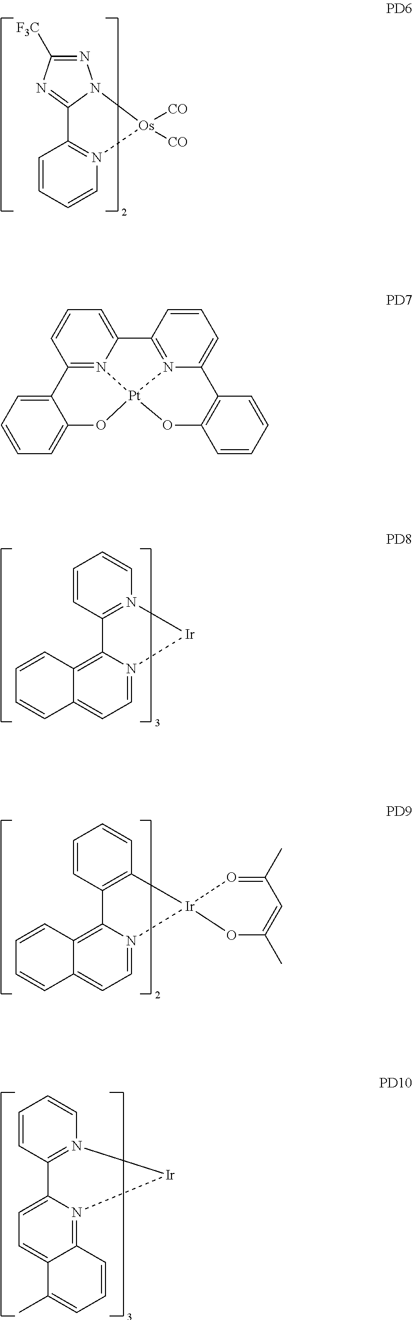

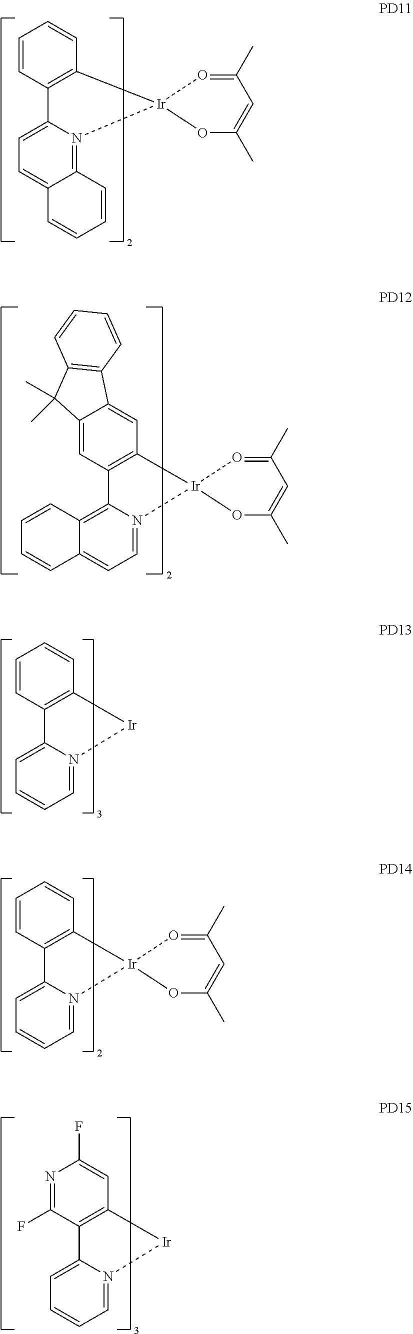

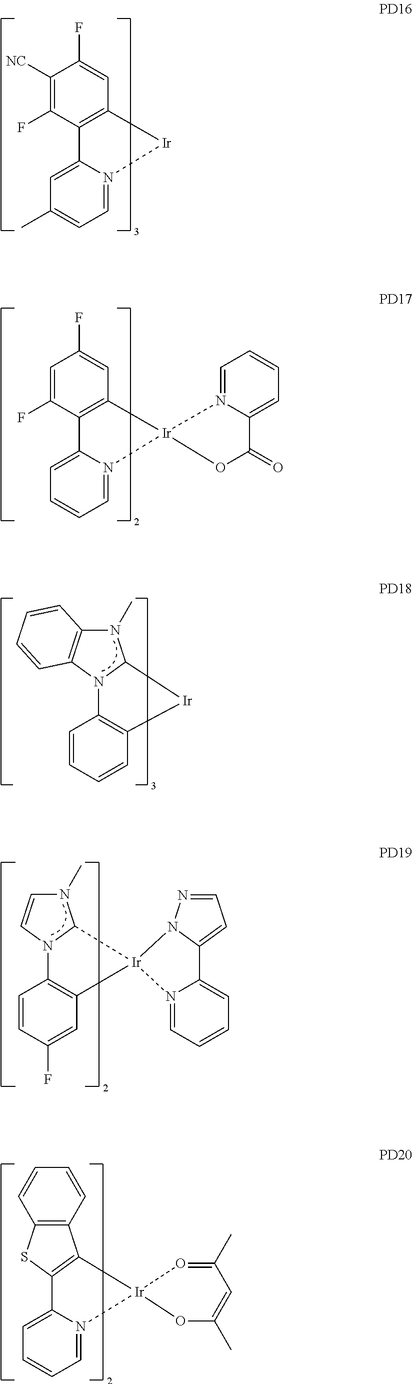

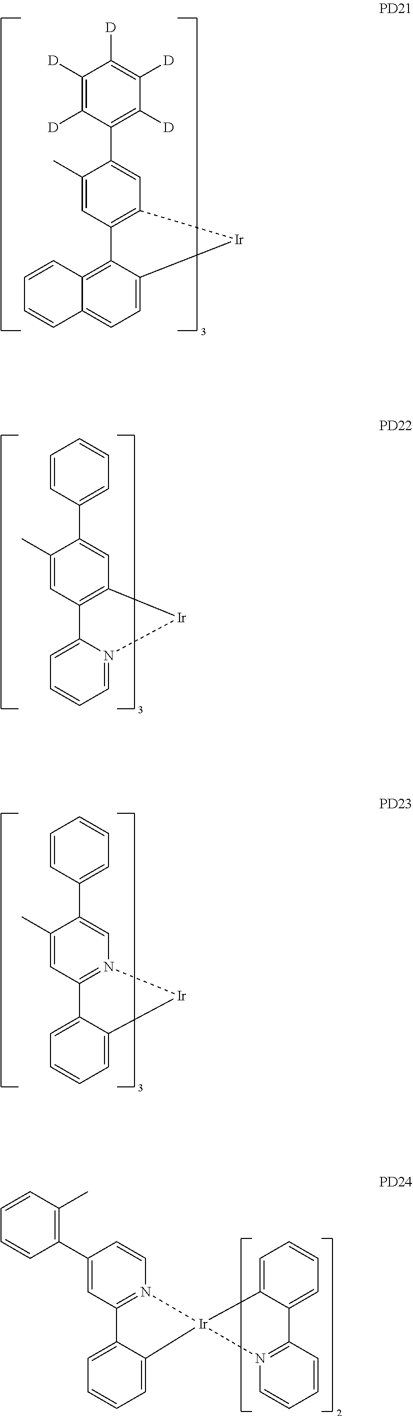

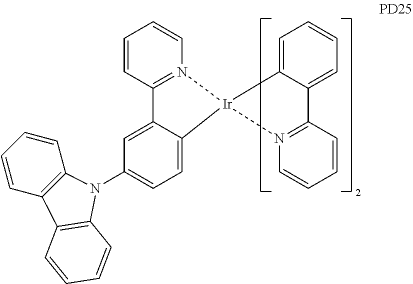

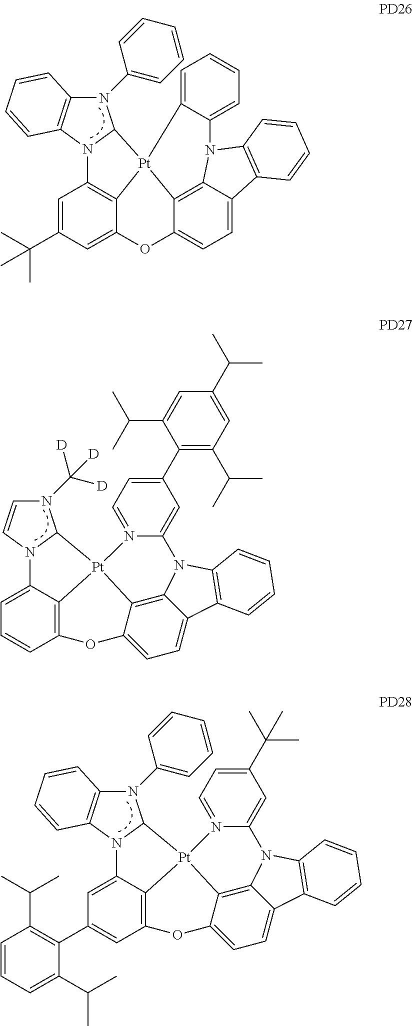

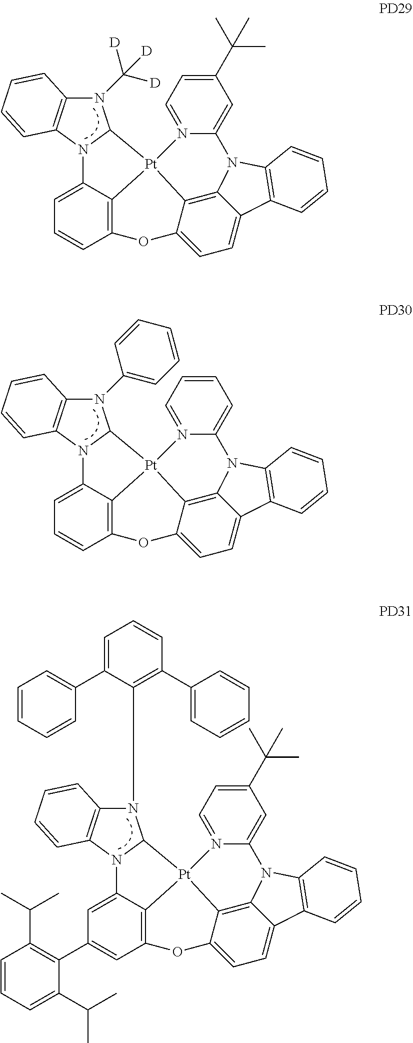

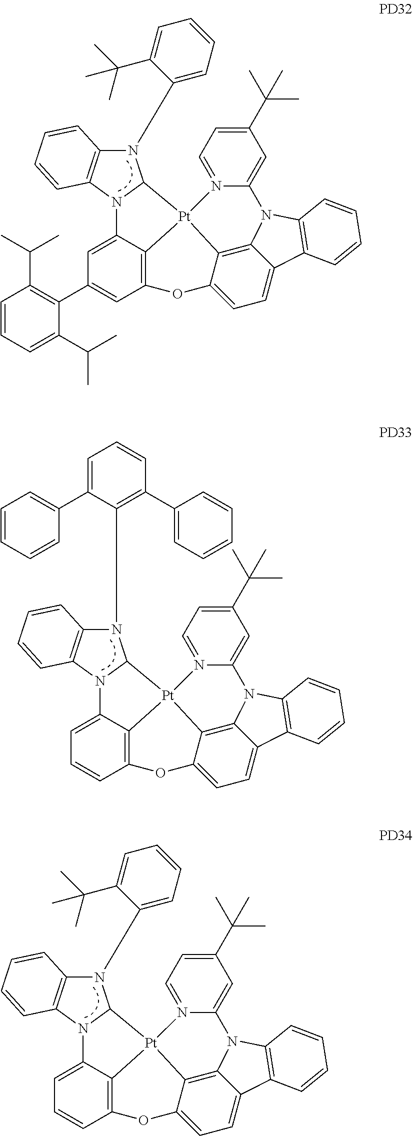

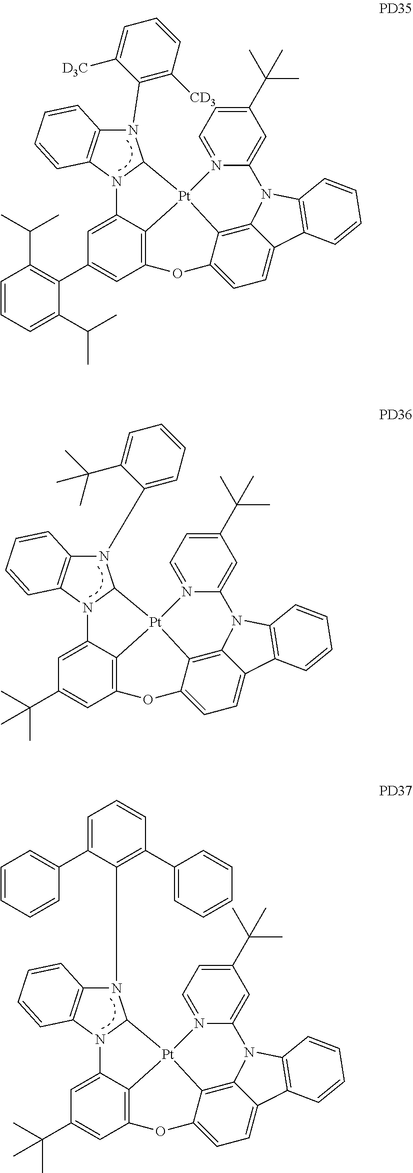

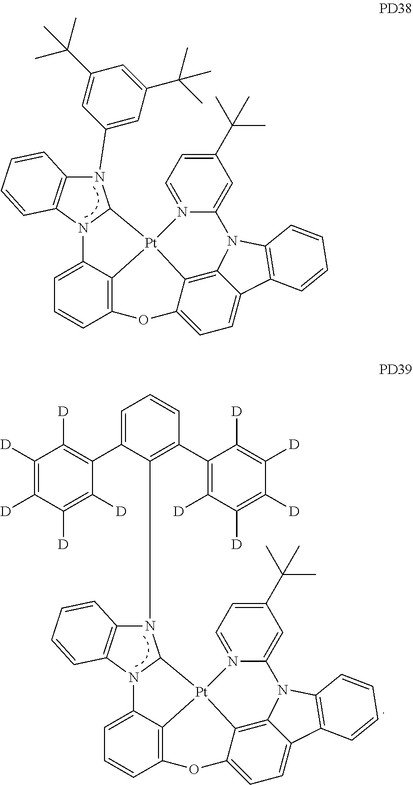

### Fluorescent Dopant

The fluorescent dopant may include an amine group-containing compound, a styryl group-containing compound, or any combination thereof.

For example, the fluorescent dopant may include a compound represented by Formula 501:

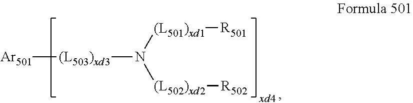

wherein, in Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

For example, Ar₅₀₁ in Formula 501 may be a condensed cyclic group (e.g., an anthracene group, a chrysene group, a pyrene group, etc.) in which three or more monocyclic groups are condensed with each other.

In one or more embodiments, xd4 in Formula 501 may be 2.

For example, the fluorescent dopant may include one or more of Compounds FD1 to FD37, DPVBi, DPAVBi, or any combination thereof:

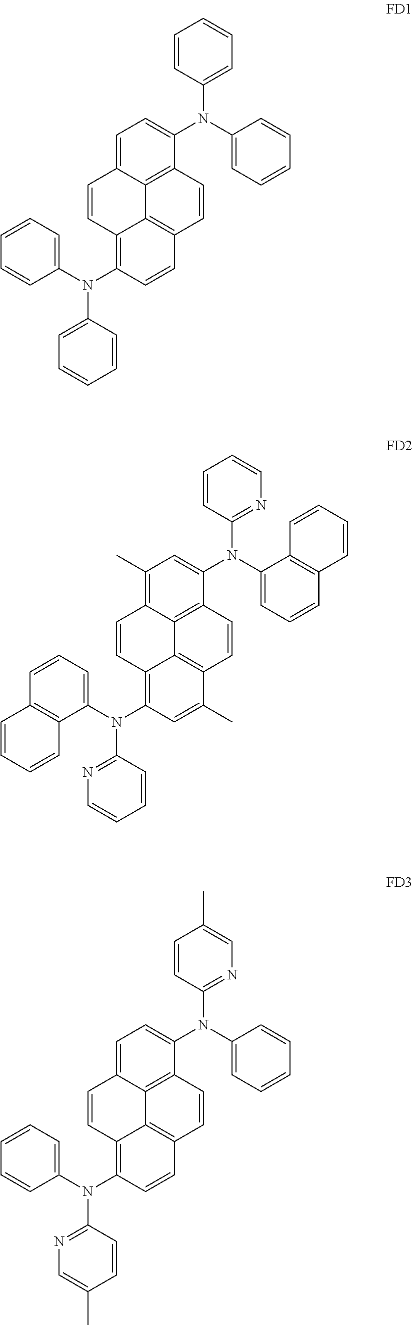 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1]

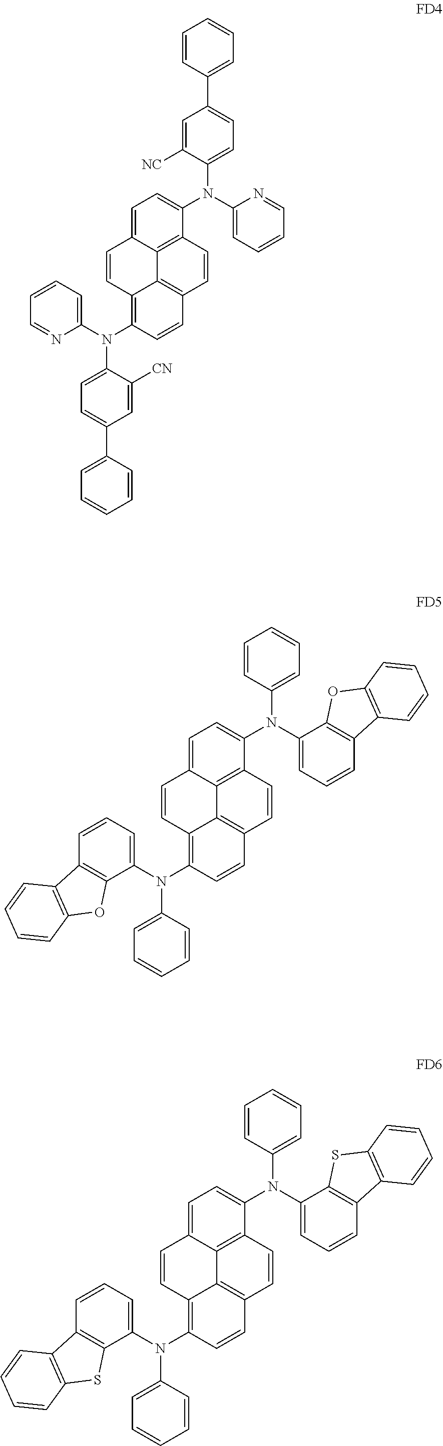 [SMILES: N#Cc1cc(-c2ccccc2)ccc1N(c1ccccn1)c1ccc2ccc3c(N(c4ccccn4)c4ccc(-c5ccccc5)cc4C#N)ccc4ccc1c2c43 ; c1ccc(N(c2ccc3ccc4c(N(c5ccccc5)c5cccc6c5oc5ccccc56)ccc5ccc2c3c54)c2cccc3c2oc2ccccc23)cc1 ; c1ccc(N(c2ccc3ccc4c(N(c5ccccc5)c5cccc6c5sc5ccccc56)ccc5ccc2c3c54)c2cccc3c2sc2ccccc23)cc1]

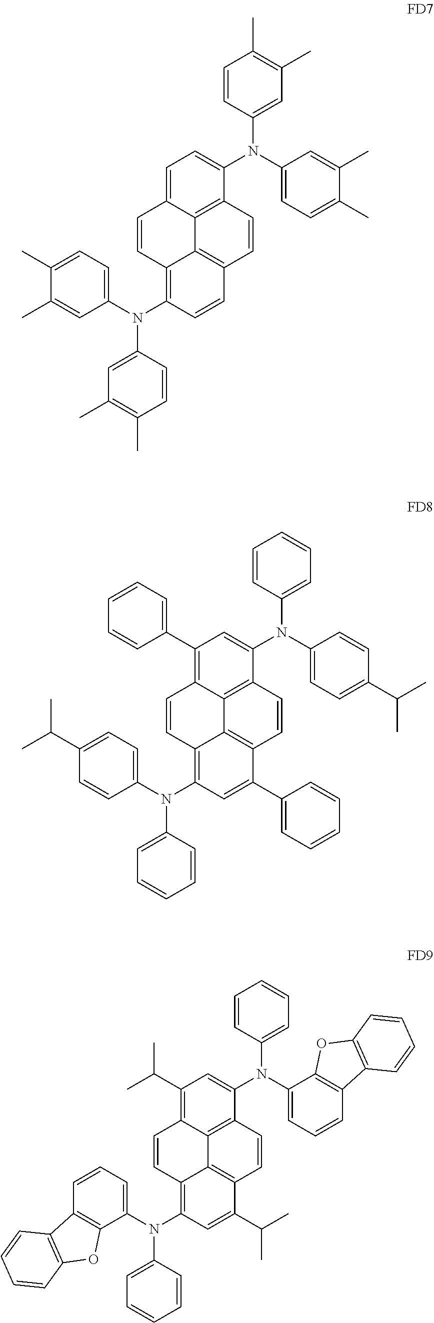

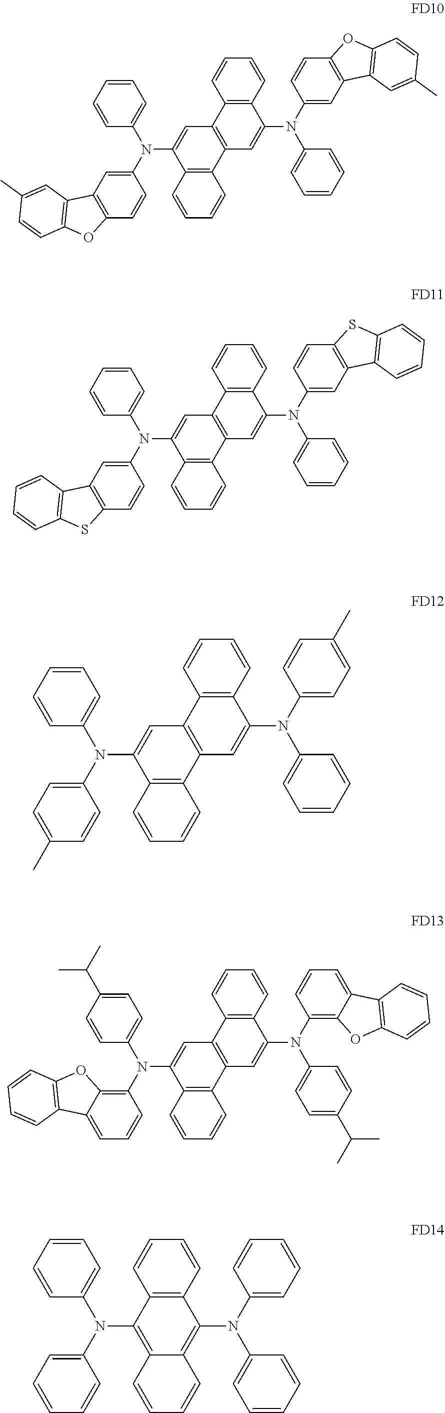

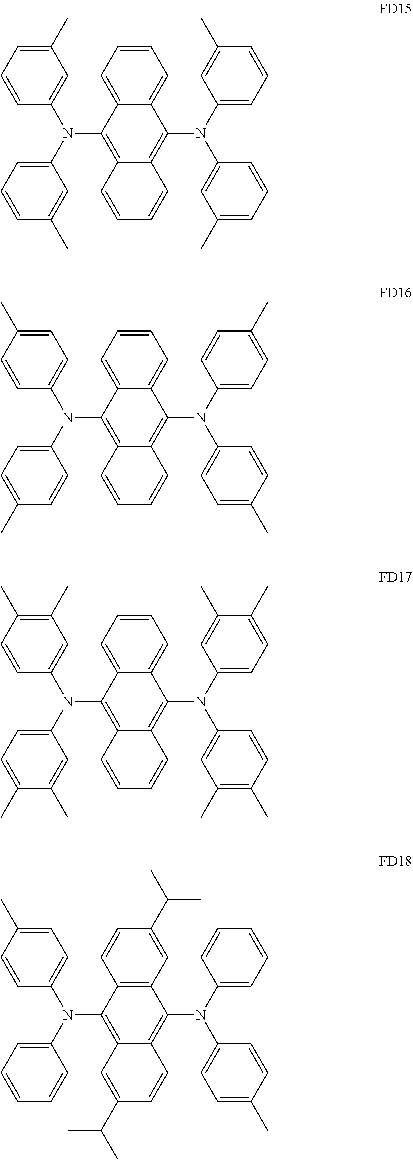 [SMILES: Cc1cccc(N(c2cccc(C)c2)c2c3ccccc3c(N(c3cccc(C)c3)c3cccc(C)c3)c3ccccc23)c1 ; Cc1ccc(N(c2ccc(C)cc2)c2c3ccccc3c(N(c3ccc(C)cc3)c3ccc(C)cc3)c3ccccc23)cc1 ; Cc1ccc(N(c2ccc(C)c(C)c2)c2c3ccccc3c(N(c3ccc(C)c(C)c3)c3ccc(C)c(C)c3)c3ccccc23)cc1C ; Cc1ccc(N(c2ccccc2)c2c3ccc(C(C)C)cc3c(N(c3ccccc3)c3ccc(C)cc3)c3ccc(C(C)C)cc23)cc1]

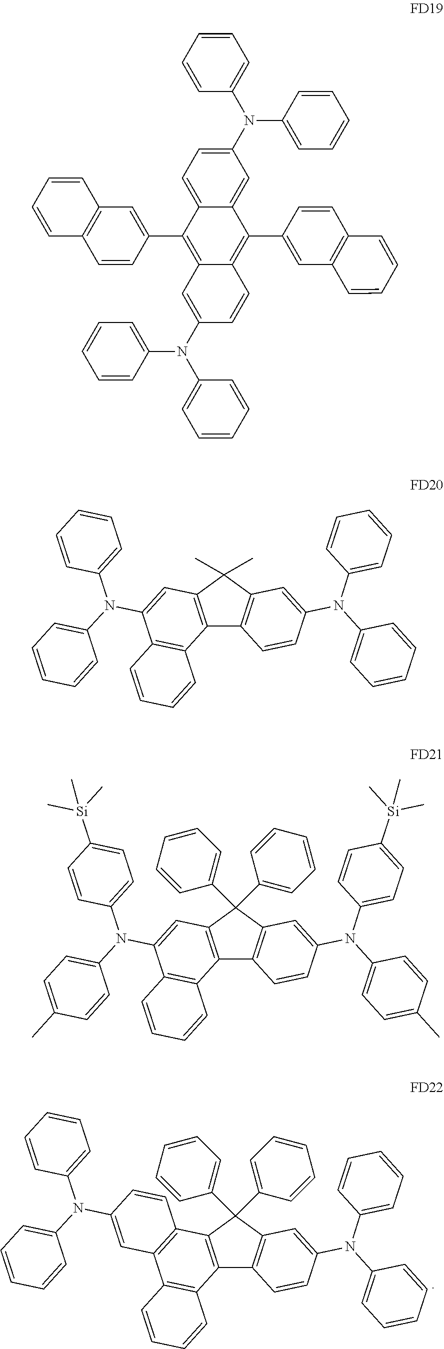

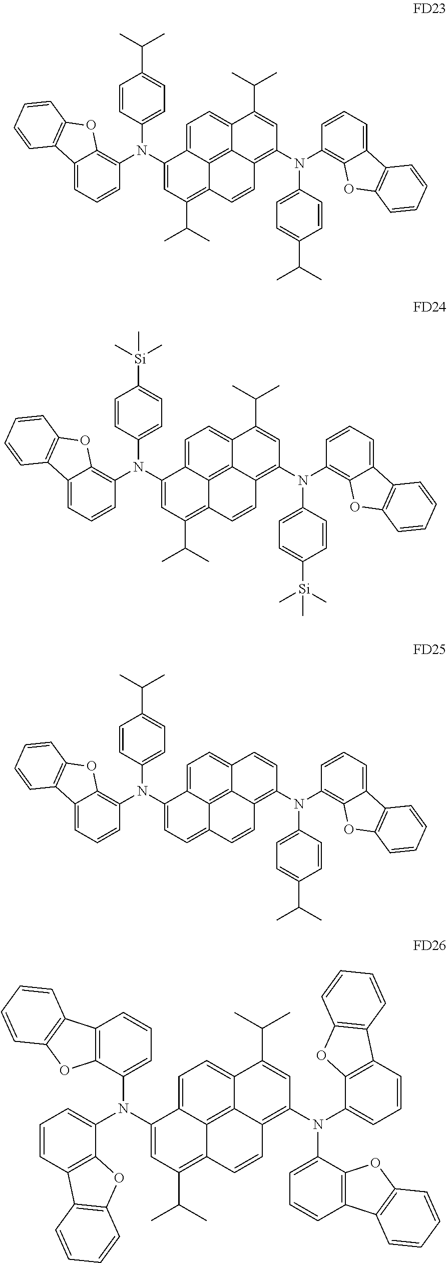 [SMILES: CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1 ; CC(C)c1cc(N(c2ccc([Si](C)(C)C)cc2)c2cccc3c2oc2ccccc23)c2ccc3c(C(C)C)cc(N(c4ccc([Si](C)(C)C)cc4)c4cccc5c4oc4ccccc45)c4ccc1c2c34 ; CC(C)c1ccc(N(c2ccc3ccc4c(N(c5ccc(C(C)C)cc5)c5cccc6c5oc5ccccc56)ccc5ccc2c3c54)c2cccc3c2oc2ccccc23)cc1 ; CC(C)c1cc(N(c2cccc3c2oc2ccccc23)c2cccc3c2oc2ccccc23)c2ccc3c(C(C)C)cc(N(c4cccc5c4oc4ccccc45)c4cccc5c4oc4ccccc45)c4ccc1c2c34]

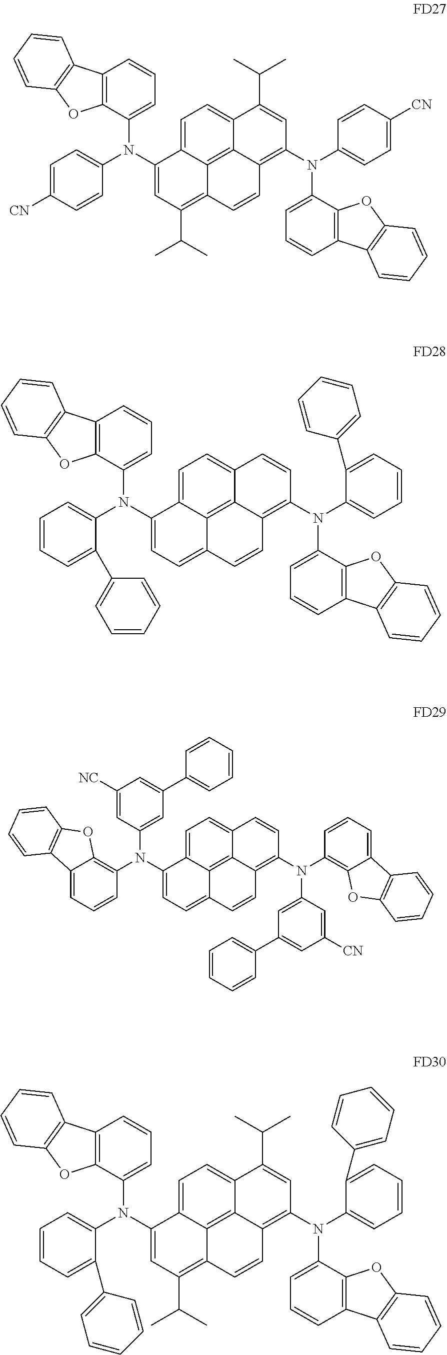

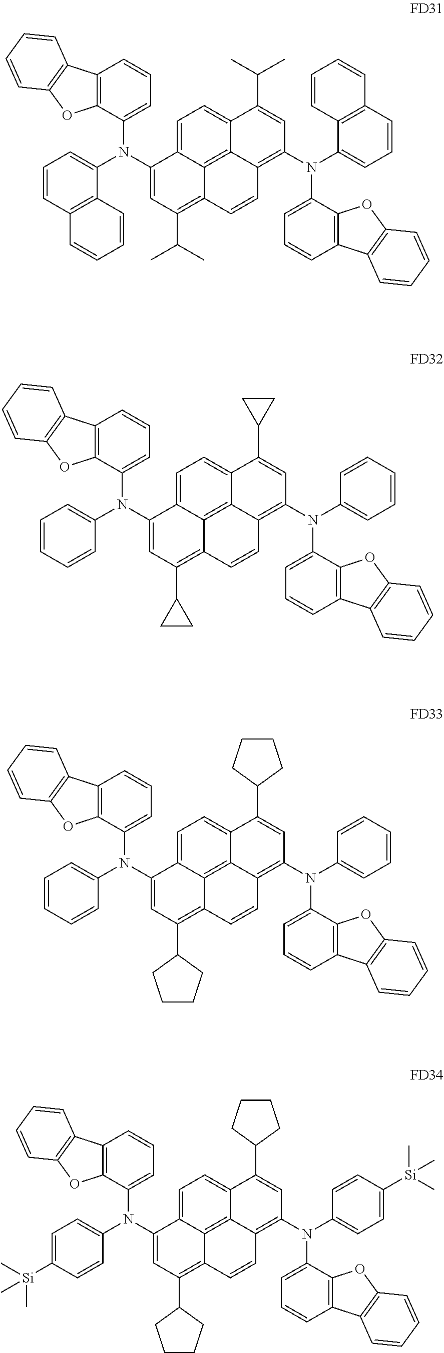

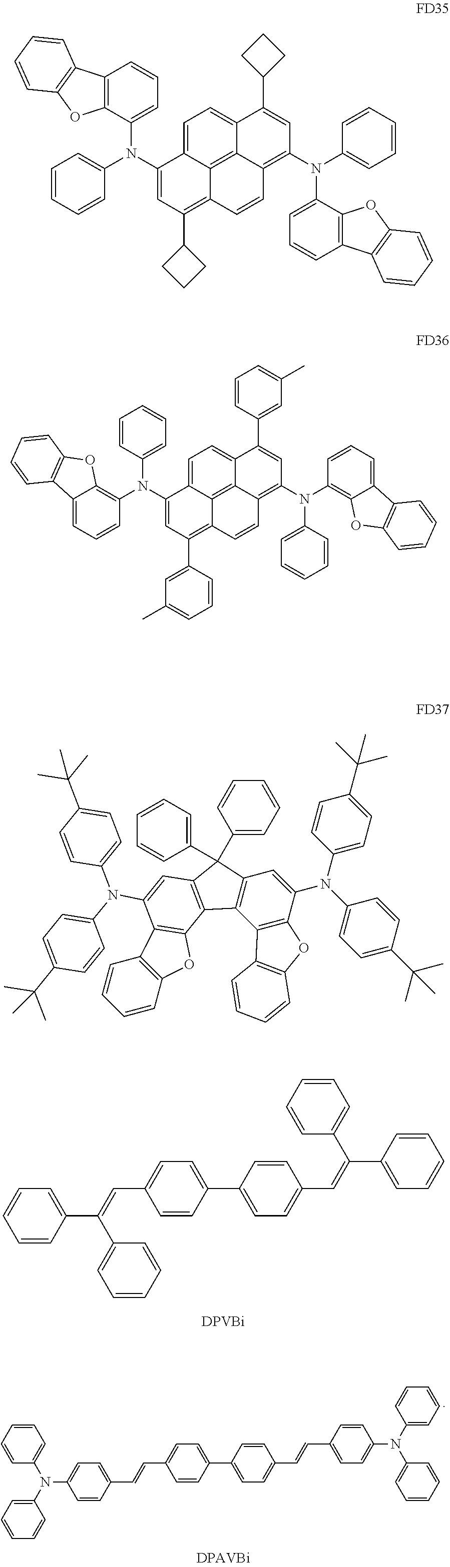

### Delayed Fluorescence Material

The emission layer 130 may include a delayed fluorescence material.

In the present specification, the delayed fluorescence material may be selected from compounds capable of emitting delayed fluorescent light based on a delayed fluorescence emission mechanism.

The delayed fluorescence material included in the emission layer 130 may serve as a host or a dopant depending on the type or kind of other materials included in the emission layer 130.

In one or more embodiments, a difference between a triplet energy level (eV) of the delayed fluorescence material and a singlet energy level (eV) of the delayed fluorescence material may be equal to or greater than 0 eV and equal to or less than 0.5 eV. When the difference between the triplet energy level (eV) of the delayed fluorescence material and the singlet energy level (eV) of the delayed fluorescence material satisfies the above-described range, up-conversion from the triplet state to the singlet state of the delayed fluorescence materials may effectively or suitably occur, and thus, the luminescence efficiency of the light-emitting device 10 may be improved.

For example, the delayed fluorescence material may include i) a material including at least one electron donor (e.g., a π electron-rich C₃-C₆₀ cyclic group, such as a carbazole group, etc.) and at least one electron acceptor (e.g., a sulfoxide group, a cyano group, a π electron-deficient nitrogen-containing C₁-C₆₀ cyclic group, etc.), and ii) a material including a C₈-C₆₀ polycyclic group in which two or more cyclic groups are condensed together while sharing boron (B).

Examples of the delayed fluorescence material may include at least one of Compounds DF1 to DF14:

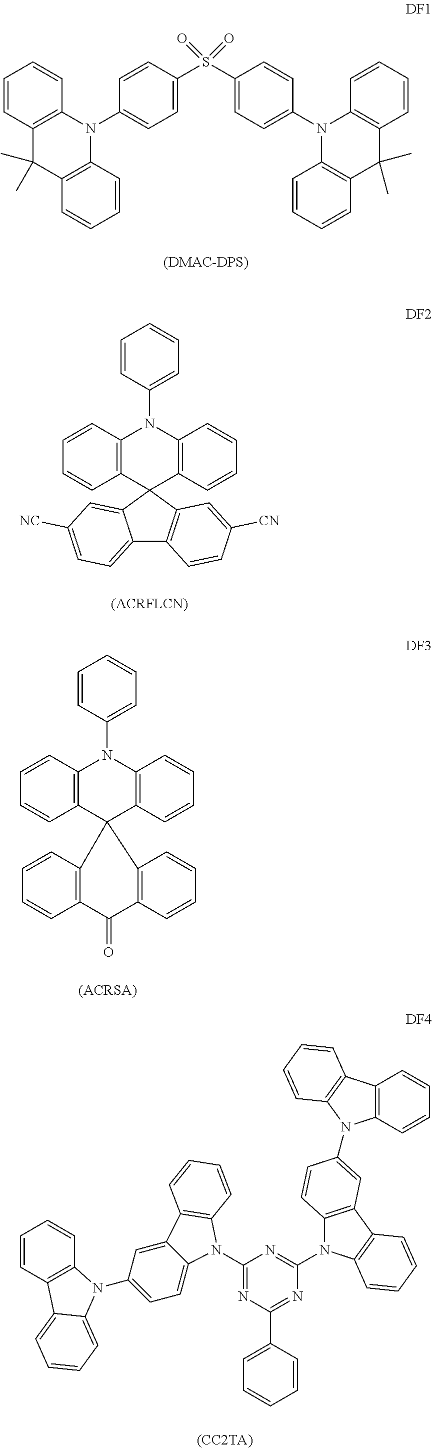

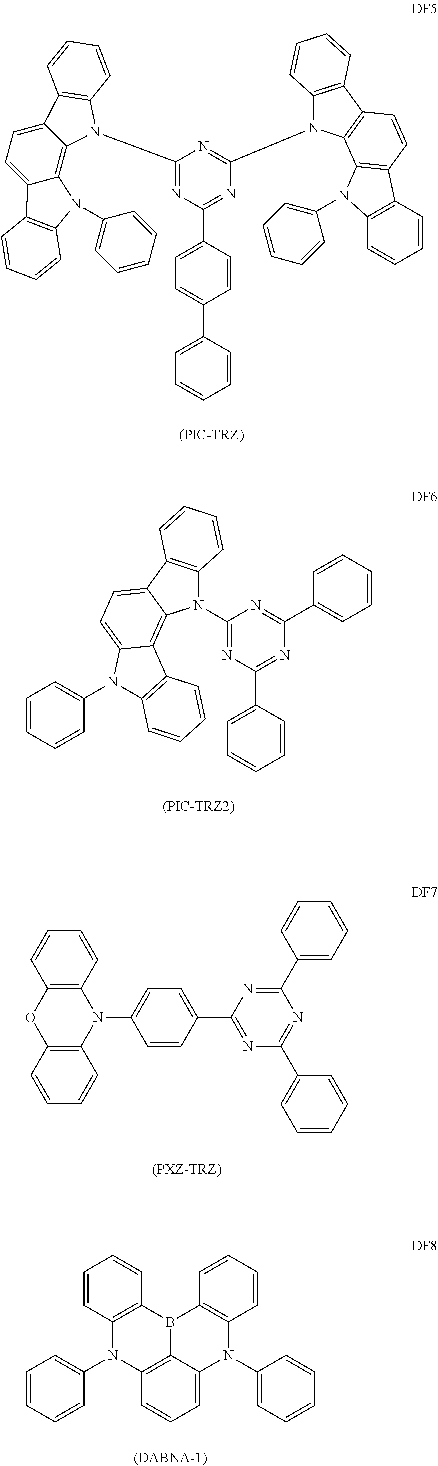

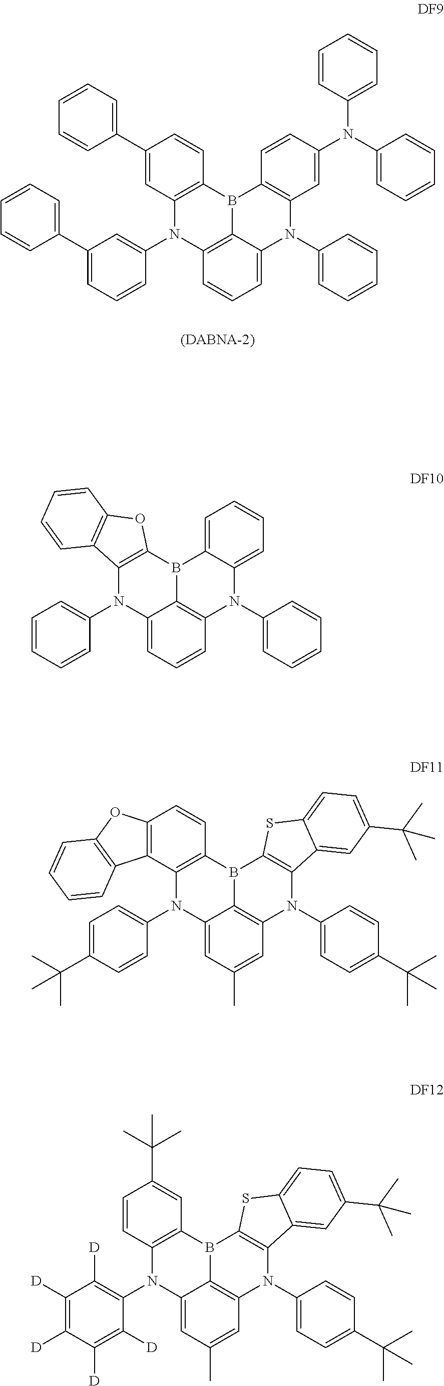

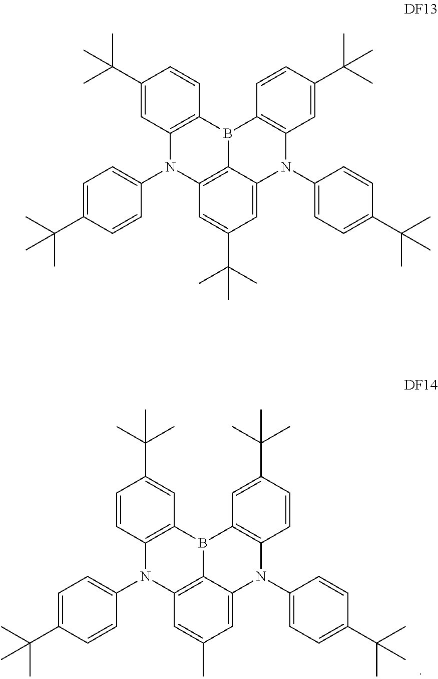

### Quantum Dot

The emission layer 130 may include a quantum dot.

The term “quantum dot” as utilized herein refers to a crystal of a semiconductor compound, and may include any material capable of emitting light of one or more suitable emission wavelengths according to the size of the crystal.

A diameter of the quantum dot may be, for example, in a range of about 1 nm to about 10 nm. In the present disclosure, when particles are spherical, “diameter” indicates a particle diameter or an average particle diameter, and when the particles are non-spherical, the “diameter” indicates a major axis length or an average major axis length. The diameter (or length) of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter (or length) is referred to as D50. D50 refers to the average diameter (or length) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

The quantum dot may be synthesized by a wet chemical process, a metal organic chemical vapor deposition process, a molecular beam epitaxy process, or any suitable process similar thereto.

The wet chemical process is a method including mixing a precursor material with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled or selected through a process which costs lower, and is easier than vapor deposition methods, such as metal organic chemical vapor deposition (MOCVD) and/or molecular beam epitaxy (MBE).

The quantum dot may include a Group II-VI semiconductor compound, a Group III-V semiconductor compound, a Group III-VI semiconductor compound, a Group I-III-VI semiconductor compound, a Group IV-VI semiconductor compound, a Group IV element or compound, or any combination thereof.

Examples of the Group II-VI semiconductor compound may include: a binary compound, such as CdS, CdSe, CdTe, ZnS, ZnSe, ZnTe, ZnO, HgS, HgSe, HgTe, MgSe, and/or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound, such as CdZnSeS, CdZnSeTe, CdZnSTe, CdHgSeS, CdHgSeTe, CdHgSTe, HgZnSeS, HgZnSeTe, and/or HgZnSTe; or any combination thereof.

Examples of the Group III-V semiconductor compound may include: a binary compound, such as GaN, GaP, GaAs, GaSb, AlN, AlP, AlAs, AlSb, InN, InP, InAs, and/or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, and/or InPSb; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, and/or InAlPSb; or any combination thereof. In one or more embodiments, the Group III-V semiconductor compound may further include a Group II element. Examples of the Group III-V semiconductor compound further including a Group II element may include InZnP, InGaZnP, InAlZnP, and/or the like.

Examples of the Group III-VI semiconductor compound may include: a binary compound, such as GaS, GaSe, Ga₂Se₃, GaTe, InS, InSe, In₂S₃, In₂Se₃, and/or InTe; a ternary compound, such as InGaS₃ and/or InGaSe₃; or any combination thereof.

Examples of the Group I-III-VI semiconductor compound may include: a ternary compound, such as AgInS, AgInS₂, CuInS, CuInS₂, CuGaO₂, AgGaO₂, and/or AgAlO₂; a quaternary compound, such as AgInGaS and/or AgInGaS₂; or any combination thereof.

Examples of the Group IV-VI semiconductor compound may include: a binary compound, such as SnS, SnSe, SnTe, PbS, PbSe, and/or PbTe; a ternary compound, such as SnSeS, SnSeTe, SnSTe, PbSeS, PbSeTe, PbSTe, SnPbS, SnPbSe, and/or SnPbTe; a quaternary compound, such as SnPbSSe, SnPbSeTe, and/or SnPbSTe; or any combination thereof.

The Group IV element or compound may include: a single element, such as Si and/or Ge; a binary compound, such as SiC and/or SiGe; or any combination thereof.

Each element included in a multi-element compound, such as the binary compound, the ternary compound, and/or the quaternary compound, may be present at a substantially uniform concentration or non-substantially uniform concentration in a particle.

In one or more embodiments, the quantum dot may have a single structure in which the concentration of each element in the quantum dot is substantially uniform, or a core-shell dual structure. For example, a material included in the core and a material included in the shell may be different from each other.

The shell of the quantum dot may act as a protective layer that prevents or reduces chemical degeneration of the core to maintain semiconductor characteristics, and/or as a charging layer that imparts electrophoretic characteristics to the quantum dot. The shell may be a single layer or a multi-layer. An interface between the core and the shell may have a concentration gradient in which the concentration of an element existing in the shell decreases toward the center of the core.

Examples of the shell of the quantum dot may include an oxide of metal, an oxide of metalloid, and/or an oxide of non-metal, a semiconductor compound, or any combination thereof. Examples of the oxide of metal, an oxide of metalloid, and/or an oxide of non-metal may include: a binary compound, such as SiO₂, Al₂O₃, TiO₂, ZnO, MnO, Mn₂O₃, Mn₃O₄, CuO, FeO, Fe₂O₃, Fe₃O₄, COO, Co₃O₄, and/or NiO; a ternary compound, such as MgAl₂O₄, CoFe₂O₄, NiFe₂O₄, and/or CoMn₂O₄; or any combination thereof. Examples of the semiconductor compound may include, as described herein, a Group II-VI semiconductor compound, a Group III-V semiconductor compound, a Group III-VI semiconductor compound, a Group I-III-VI semiconductor compound, a Group IV-VI semiconductor compound, or any combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of an emission wavelength spectrum of the quantum dot may be about 45 nm or less, for example, about 40 nm or less, for example, about 30 nm or less, and within any of these ranges, color purity and/or color reproducibility may be improved. In some embodiments, because the light emitted through the quantum dot is emitted in all directions, the viewing angle of light may be improved.

In some embodiments, the quantum dot may be in the form of a spherical particle, a pyramidal particle, a multi-arm particle, a cubic nanoparticle, a nanotube particle, a nanowire particle, a nanofiber particle, and/or a nanoplate particle.

Because the energy band gap may be adjusted by controlling the size of the quantum dot, light having various wavelength bands may be obtained from an emission layer including the quantum dot. Accordingly, by utilizing quantum dots of different sizes, a light-emitting device that emits light of one or more suitable wavelengths may be implemented. In some embodiments, the size of the quantum dot may be selected to emit red, green and/or blue light. In some embodiments, the size of the quantum dot may be configured to emit white light by combining light of various colors.

### Photoactive Layer 135

The opto-electronic device 30 may include the photoactive layer 135 arranged on the hole transport region 120. The photoactive layer 135 may be arranged between the hole transport region 120 and the electron transport region 140. In one or more embodiments, the photoactive layer 135 may be arranged between the hole transport layer included in the hole transport region 120 and the buffer layer included in the electron transport region 140. In one or more embodiments, the photoactive layer 135 may be arranged between the emission auxiliary layer included in the hole transport region 120 and the buffer layer included in the electron transport region 140.

The photoactive layer 135 may include the organic compound and the electron accepting compound. In some embodiments, the organic compound and the electron accepting compound may be mixed and included in the photoactive layer 135. For example, the photoactive layer 135 may be a single layer including the organic compound and the electron accepting compound.

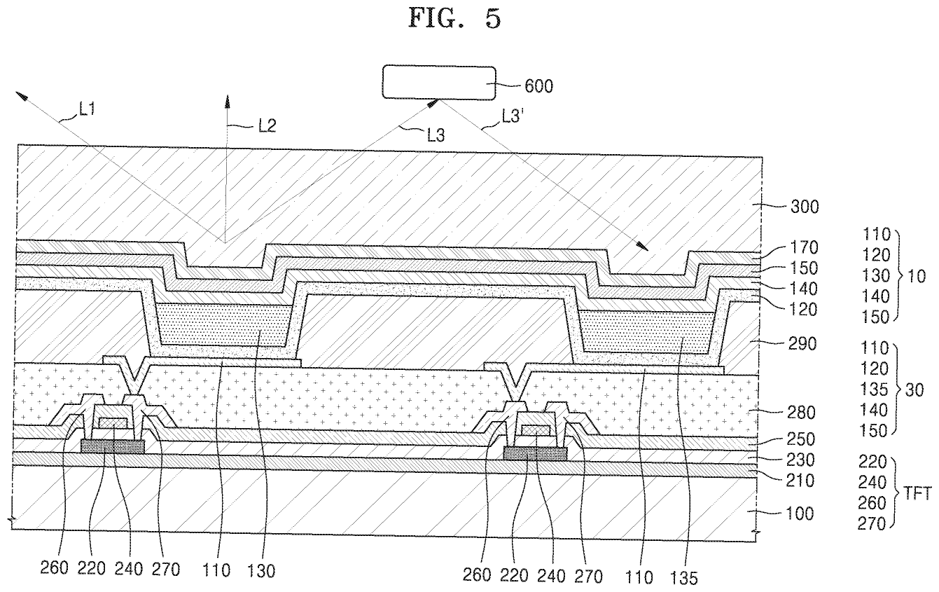

The photoactive layer 135 may absorb light incident to the electronic apparatus to form excitons. The excitons may generate holes and electrons. For example, the photoactive layer 135 may absorb light to generate an electrical signal. In one or more embodiments, the organic compound included in the photoactive layer 135 may serve as a donor for supplying electrons, and the electron accepting compound included in the photoactive layer 135 may serve as an acceptor for receiving electrons. Accordingly, the opto-electronic device 30 including the photoactive layer 135 may serve as a photosensor. For example, the opto-electronic device 30 may serve as a fingerprint recognition sensor, which will be described in more detail herein below with reference to FIG. 5.

### Electron Transport Region 140

The electron transport region 140 may have i) a single-layered structure including (e.g., consisting of) a single layer including (e.g., consisting of) a single material, ii) a single-layered structure including (e.g., consisting of) a single layer including a plurality of different materials, or iii) a multi-layered structure including a plurality of layers including different materials.

The electron transport region 140 may include a buffer layer, a hole blocking layer, an electron control layer, an electron transport layer, an electron injection layer, or any combination thereof.

For example, the electron transport region 140 may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, the layers of each structure being sequentially stacked from the emission layer 130.

The electron transport region 140 (e.g., the buffer layer, the hole blocking layer, the electron control layer, and/or the electron transport layer in the electron transport region 140) may include a metal-free compound including at least one π electron-deficient nitrogen-containing C₁-C₆₀ cyclic group.

For example, the electron transport region 140 may include a compound represented by Formula 601:

[Ar₆₀₁]ₓₑ₁₁-[(L₆₀₁)ₓₑ₁-R₆₀₁]ₓₑ₂₁, Formula 601
wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each be as defined herein in connection with Q₁,
xe21 may be 1, 2, 3, 4, or 5, and
at least one selected from Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In one or more embodiments, Ar₆₀₁ in Formula 601 may be a substituted or unsubstituted anthracene group.

In one or more embodiments, the electron transport region 140 may include a compound represented by Formula 601-1:

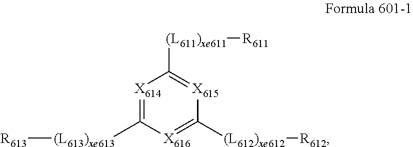

wherein, in Formula 601-1,
X₆₁₄ may be N or C(R⁶¹⁴), X₆₀₅ may be N or C(R₆₁₅), X₆₁₆ may be N or C(R₆₁₆), and at least one of X₆₁₄ to X₆₁₆ may be N,
L₆₁₁ to L₆₁₃ may each be as defined herein in connection with L₆₀₁,
xe611 to xe613 may each be as defined herein in connection with xe1,
R₆₁₁ to R₆₁₃ may each be as defined herein in connection with R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region 140 may include one or more of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

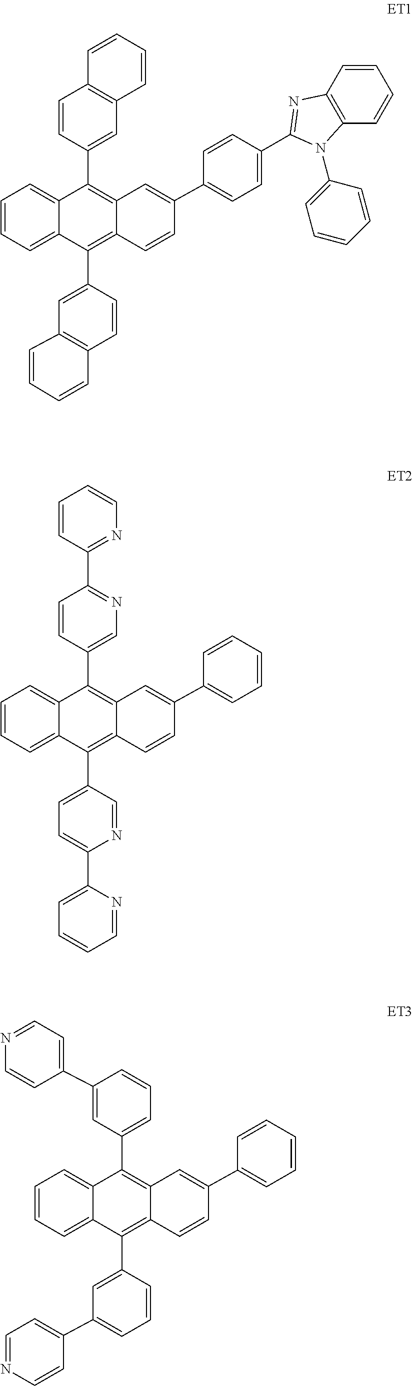 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

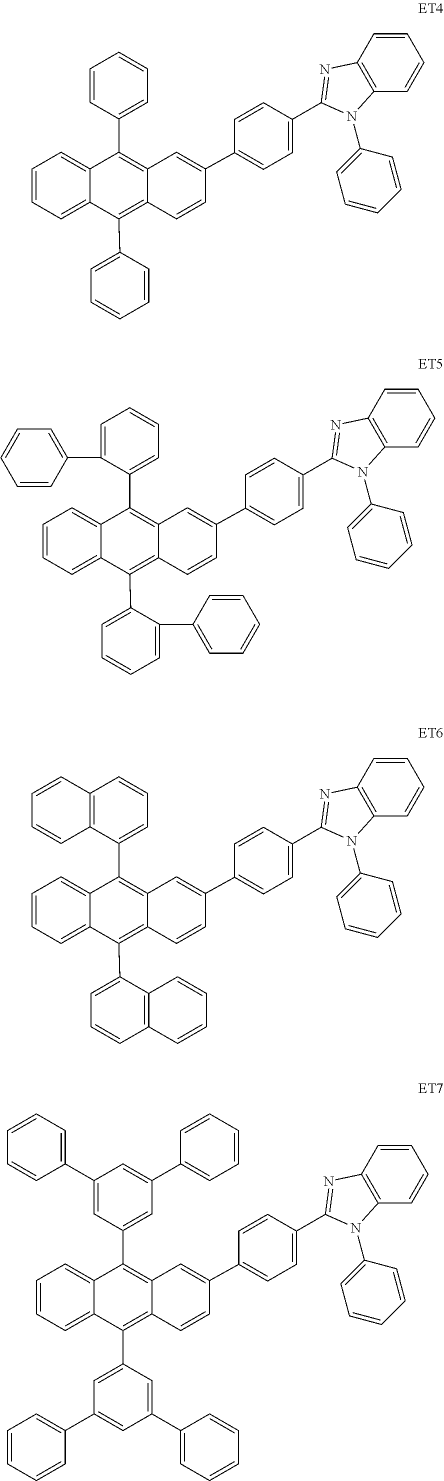 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

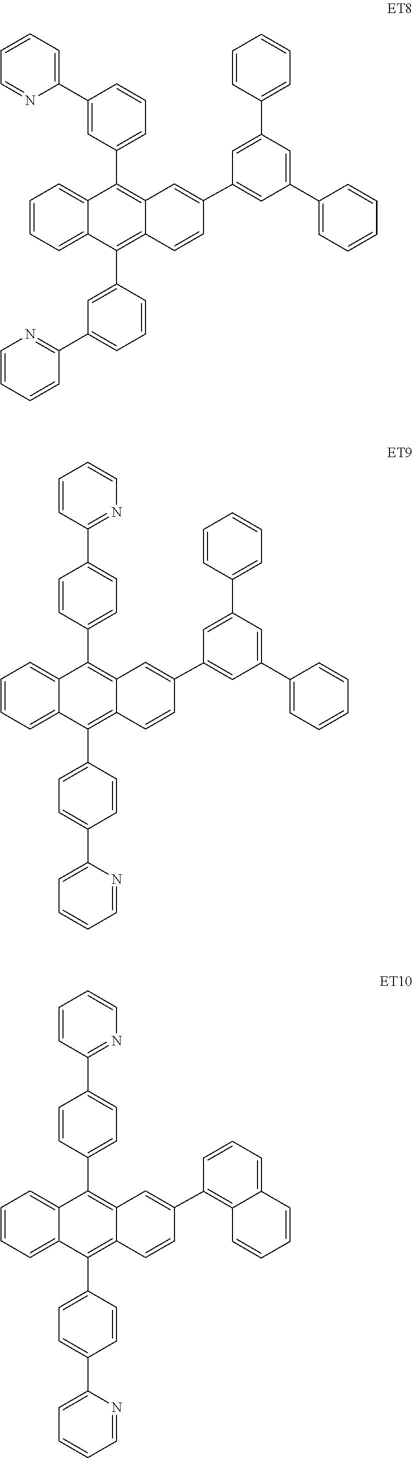 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

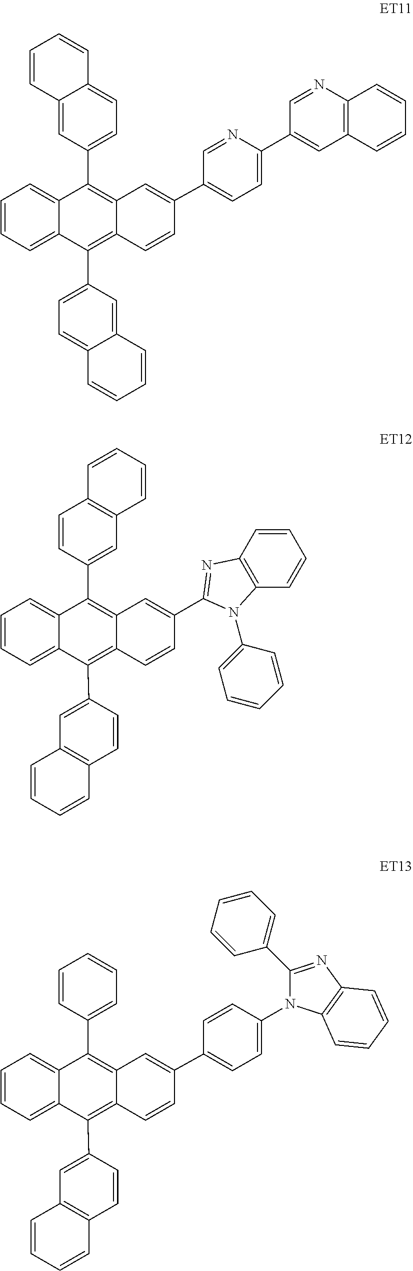 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

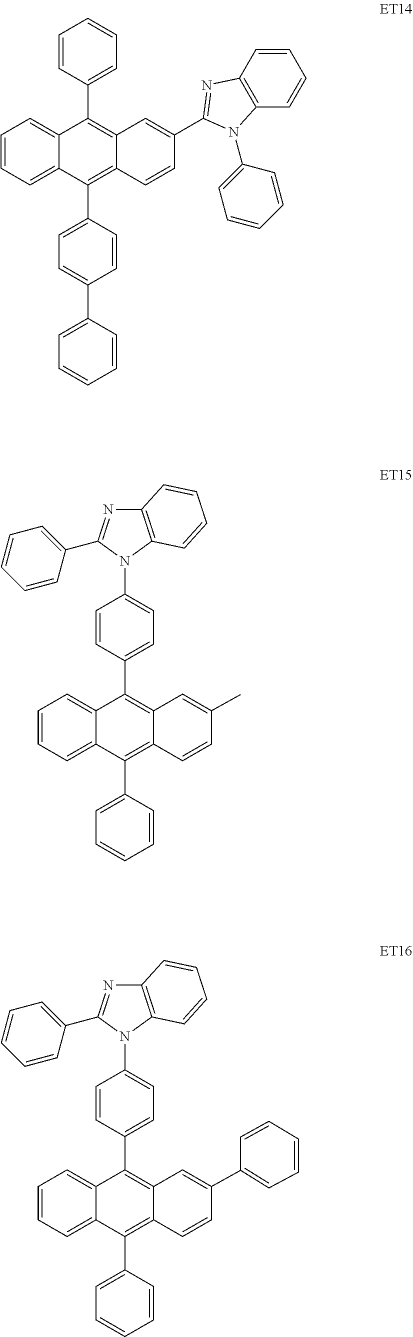 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

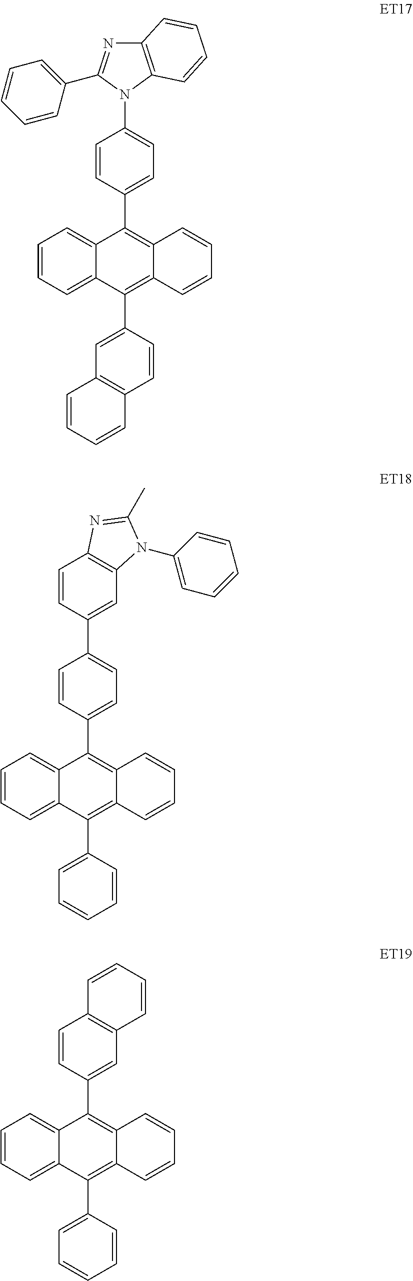

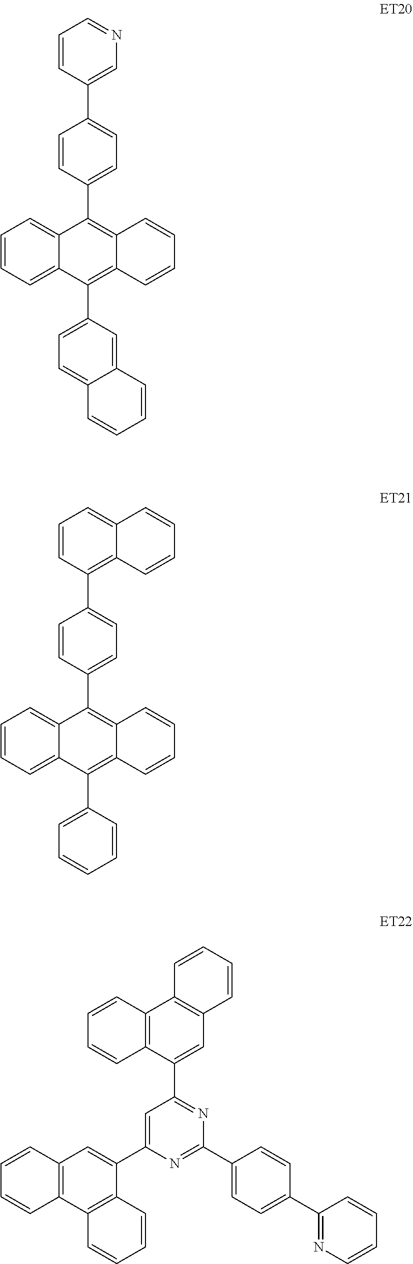

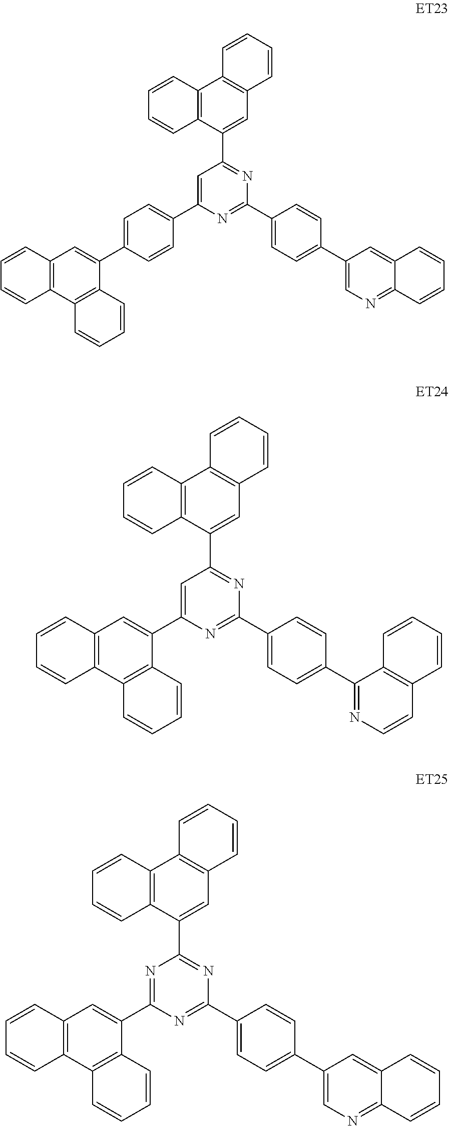 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

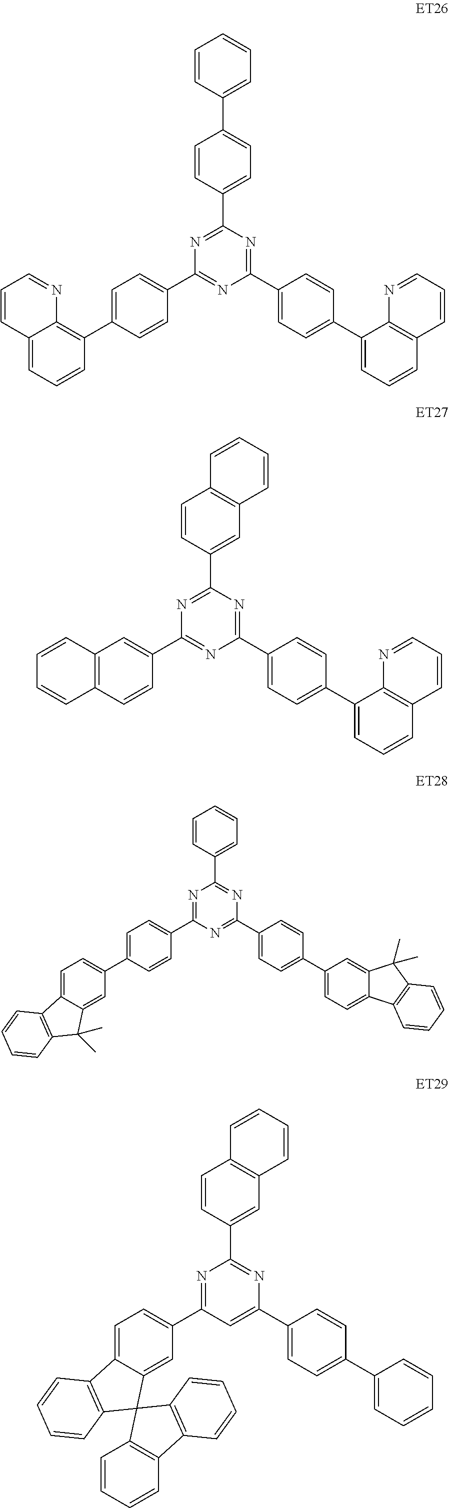 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

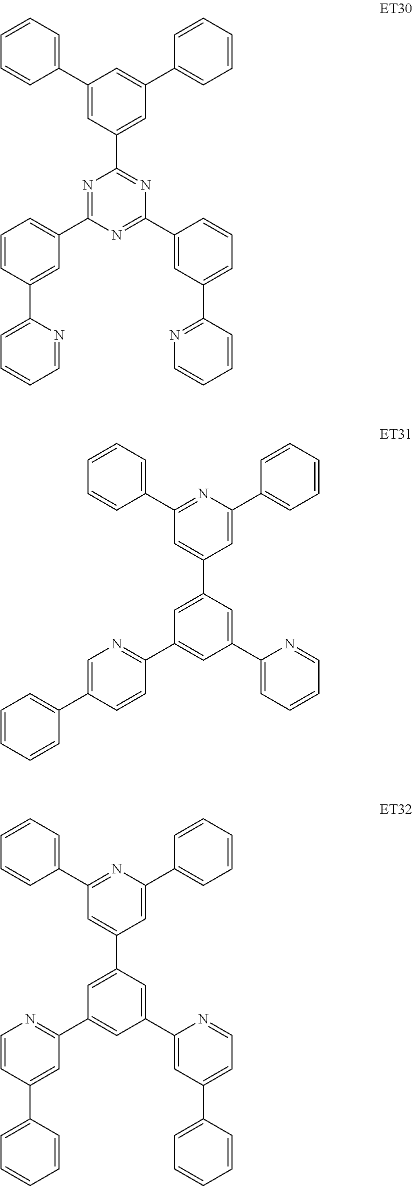 [SMILES: c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

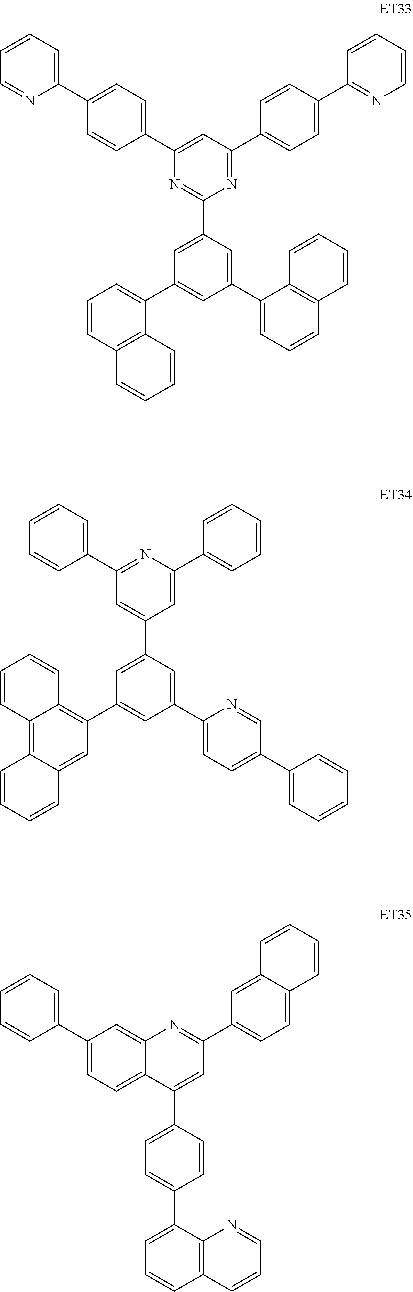 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1]

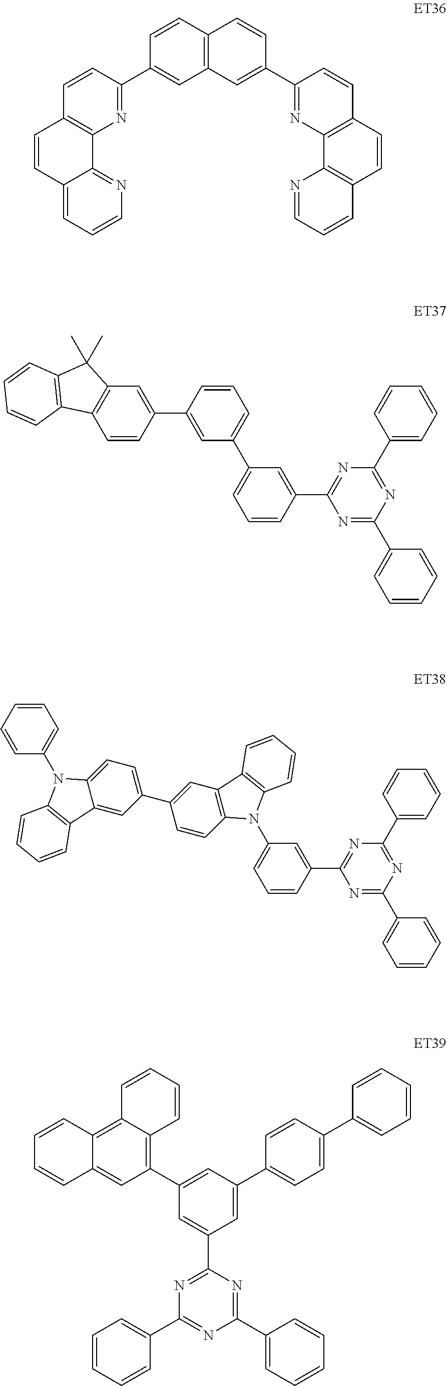 [SMILES: c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1]

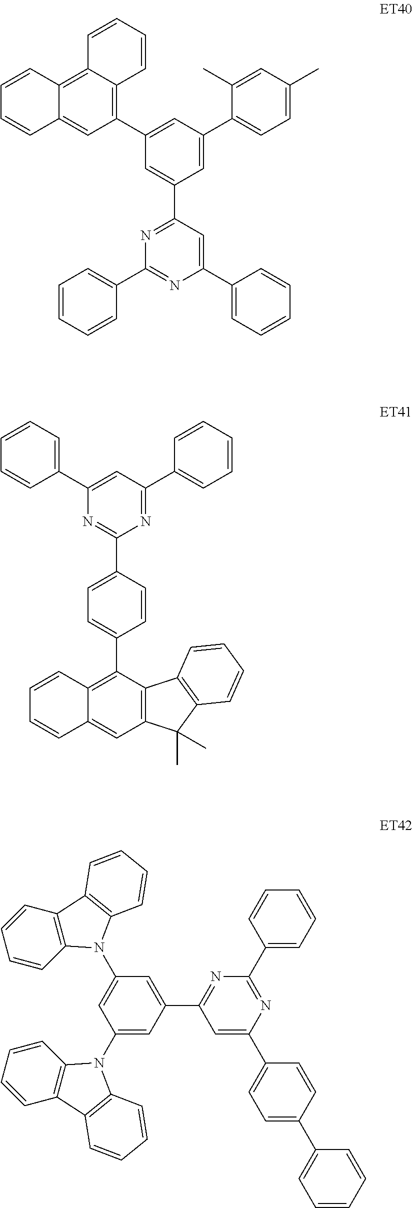 [SMILES: Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

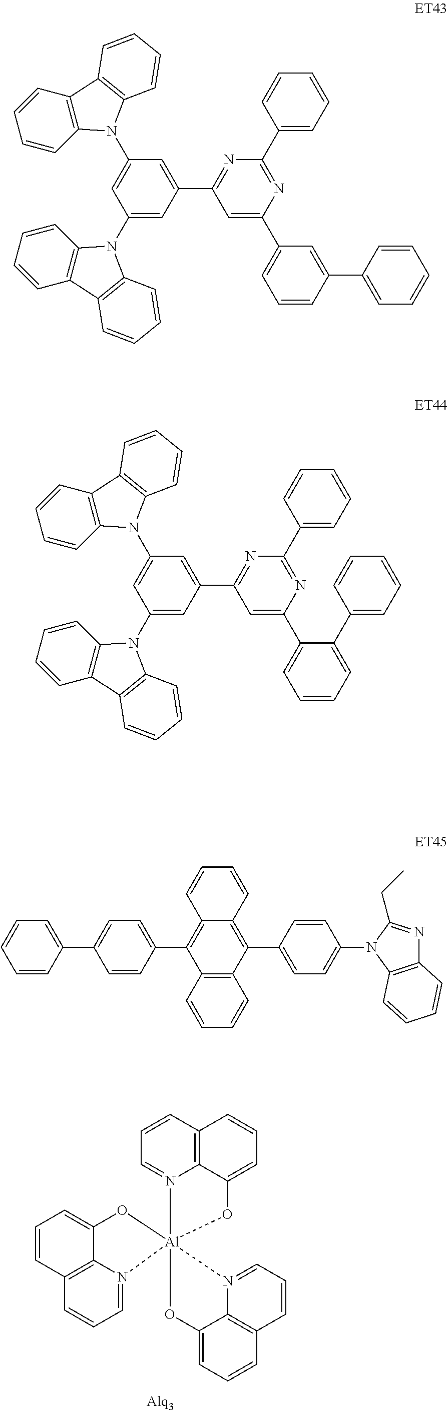

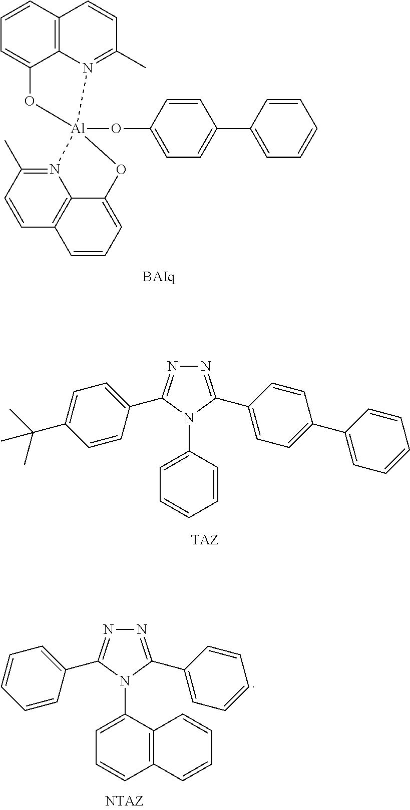

A thickness of the electron transport region 140 may be from about 100 Å to about 5,000 Å, for example, about 160 Å to about 4,000 Å. When the electron transport region 140 includes a buffer layer, a hole blocking layer, an electron control layer, an electron transport layer, or any combination thereof, a thickness of the buffer layer, the hole blocking layer, and/or the electron control layer may each independently be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. When the thicknesses of the buffer layer, the hole blocking layer, the electron control layer, the electron transport layer, and/or the electron transport region 140 are within any of their respective ranges, satisfactory or suitable electron transporting characteristics may be obtained without a substantial increase in driving voltage.

The electron transport region 140 (e.g., the electron transport layer in the electron transport region 140) may further include, in addition to the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, and/or a Cs ion, and a metal ion of the alkaline earth metal complex may be a Be ion, a Mg ion, a Ca ion, a Sr ion, and/or a Ba ion. A ligand coordinated with the metal ion of the alkali metal complex and/or the alkaline earth-metal complex may include hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or any combination thereof.

For example, the metal-containing material may include a Li complex. The Li complex may include, for example, Compound ET-D1 (LiQ) and/or Compound ET-D2:

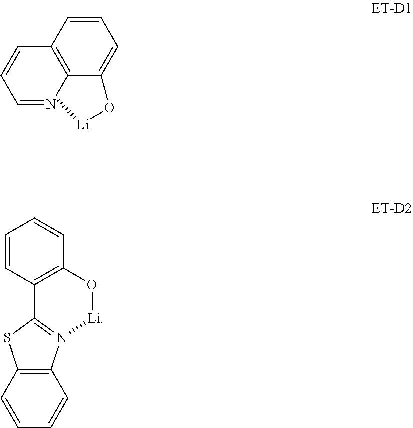

The electron transport region 140 may include an electron injection layer that facilitates injection of electrons from the second electrode 150. The electron injection layer may be in direct contact with the second electrode 150.

The electron injection layer may have i) a single-layered structure including (e.g., consisting of) a single layer including (e.g., consisting of) a single material, ii) a single-layered structure including (e.g., consisting of) a single layer including a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include oxides, halides (e.g., fluorides, chlorides, bromides, iodides, etc.), and/or tellurides of the alkali metal, the alkaline earth metal, and the rare earth metal, or any combination thereof.

The alkali metal-containing compound may include an alkali metal oxide, such as Li₂O, Cs₂O, and/or K₂O, an alkali metal halide, such as LiF, NaF, CsF, KF, LiI, NaI, CsI, and/or KI, or any combination thereof. The alkaline earth metal-containing compound may include an alkaline earth metal compound, such as BaO, SrO, CaO, BaₓSr₁₋ₓO (wherein x is a real number satisfying the condition of 0<x<1), and/or BaₓCa₁₋ₓO (wherein x is a real number satisfying the condition of 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include a lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and/or the like.

The alkali metal complex, the alkaline earth-metal complex, and the rare earth metal complex may include i) one of ions of the alkali metal, the alkaline earth metal, and the rare earth metal and ii), as a ligand linked to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (e.g., a compound represented by Formula 601).

In one or more embodiments, the electron injection layer may include (e.g., consist of) i) an alkali metal-containing compound (e.g., an alkali metal halide), or ii) a) an alkali metal-containing compound (e.g., an alkali metal halide); and b) an alkali metal, an alkaline earth metal, a rare earth metal, or any combination thereof. For example, the electron injection layer may be a KI:Yb co-deposited layer, a RbI:Yb co-deposited layer, a LiF:Yb co-deposited layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be substantially uniformly or substantially non-uniformly dispersed in a matrix including the organic material.

A thickness of the electron injection layer may be in a range of about 1 Å to about 100 Å, for example, about 3 Å to about 90 Å. When the thickness of the electron injection layer is within any of these ranges, satisfactory or suitable electron injection characteristics may be obtained without a substantial increase in driving voltage.

### Second Electrode 150

The second electrode 150 may be arranged on the electron transport region 140. The second electrode 150 may be a cathode, which is an electron injection electrode, and as the material for the second electrode 150, a metal, an alloy, an electrically conductive compound, or any combination thereof, each having a low work function, may be utilized.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

### Capping Layer

A first capping layer may be arranged outside the first electrode 110, and/or a second capping layer may be arranged outside the second electrode 150. In detail, the light-emitting device 10 may have a structure in which the first capping layer, the first electrode 110, the emission layer 130, and the second electrode 150 are sequentially stacked in the stated order, a structure in which the first electrode 110, the emission layer 130, the second electrode 150, and the second capping layer are sequentially stacked in the stated order, or a structure in which the first capping layer, the first electrode 110, the emission layer 130, the second electrode 150, and the second capping layer are sequentially stacked in the stated order.

Light generated in the emission layer 130 of the light-emitting device 10 may be extracted toward the outside through the first electrode 110, which is a semi-transmissive electrode or a transmissive electrode, and the first capping layer, or light generated in the emission layer 130 of the light-emitting device 10 may be extracted toward the outside through the second electrode 150, which is a semi-transmissive electrode or a transmissive electrode, and the second capping layer.

The first capping layer and the second capping layer may increase external luminescence efficiency according to the principle of constructive interference. Accordingly, the light extraction efficiency of the light-emitting device 10 may be increased, so that the luminescence efficiency of the light-emitting device 10 may be improved.

Each of the first capping layer and the second capping layer may include a material having a refractive index of about 1.6 or more (at 589 nm).

The first capping layer and the second capping layer may each independently be an organic capping layer including an organic material, an inorganic capping layer including an inorganic material, or an organic-inorganic composite capping layer including an organic material and an inorganic material.

At least one selected from the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and/or the amine group-containing compound may optionally be substituted with a substituent including O, N, S, Se, Si, F, Cl, Br, I, or any combination thereof. In one or more embodiments, at least one selected from the first capping layer and the second capping layer may each independently include an amine group-containing compound.

For example, at least one selected from the first capping layer and the second capping layer may each independently include a compound represented by Formula 201, a compound represented by Formula 202, or any combination thereof.

In one or more embodiments, at least one selected from the first capping layer and the second capping layer may each independently include one or more of Compounds HT28 to HT33, one or more of Compounds CP1 to CP6, β-NPB, or any combination thereof:

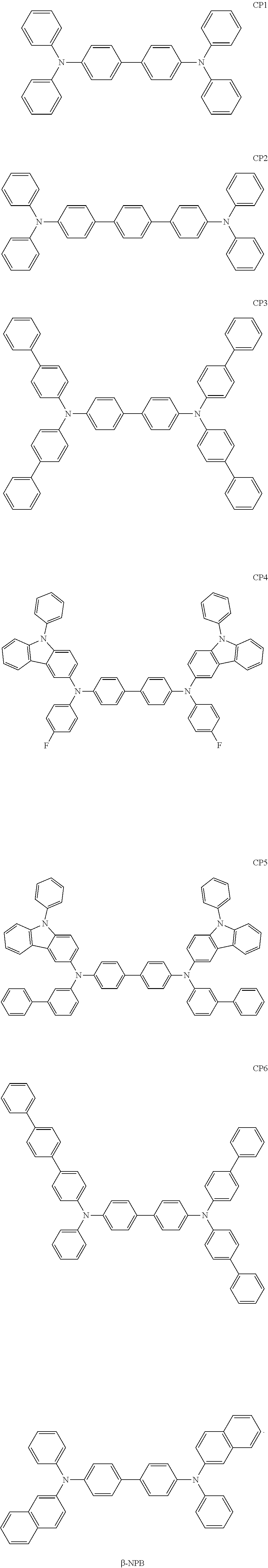

### Film

According to one or more embodiments of the disclosure, the electronic apparatus may include a film. The film may be, for example, an optical member (and/or a light control member) (e.g., a color filter, a color conversion member, a capping layer, a light extraction efficiency improvement layer, a selective light absorption layer, a polarizing layer, a quantum dot-containing layer, etc.), a light blocking member (e.g., a light reflection layer, a light absorption layer, etc.), a protection member (e.g. an insulating layer, a dielectric layer, etc.), and/or the like.

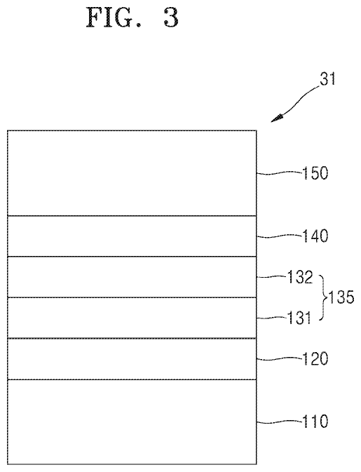

### Description for FIG. 3

FIG. 3 is a schematic view of an opto-electronic device 31 according to one or more embodiments.

The opto-electronic device 31 shown in FIG. 3 is the same as and/or similar to the opto-electronic device 30 shown in FIG. 1, except for the photoactive layer 135, and thus, descriptions of other components are omitted (e.g., are not provided).

The opto-electronic device 31 may include the photoactive layer 135 arranged between the hole transport region 120 and the electron transport region 140. In one or more embodiments, the photoactive layer 135 may be arranged between the hole transport layer included in the hole transport region 120 and the buffer layer included in the electron transport region 140. In one or more embodiments, the photoactive layer 135 may be arranged between the emission auxiliary layer included in the hole transport region 120 and the buffer layer included in the electron transport region 140.

The photoactive layer 135 may include a first layer 131 adjacent to the hole transport region 120 and a second layer 132 adjacent to the electron transport region 140. In one or more embodiments, the first layer 131 may be in direct contact with the second layer 132.

In one or more embodiments, the first layer 131 may be in direct contact with the hole transport layer included in the hole transport region 120. In one or more embodiments, the first layer 131 may be in direct contact with the emission auxiliary layer arranged on the hole transport layer.

In one or more embodiments, the second layer 132 may be in direct contact with the buffer layer included in the electron transport region 140.

The first layer 131 may include the organic compound. For example, the first layer 131 may not include (e.g., may exclude) the electron accepting compound.

The second layer 132 may include the electron accepting compound. For example, the second layer 132 may not include (e.g., may exclude) the organic compound.

For example, the photoactive layer 135 may have a multi-layered structure divided into the first layer 131 including the organic compound and the second layer 132 including the electron accepting compound.

The photoactive layer 135 may absorb incident light to form excitons. The excitons may generate holes and electrons. For example, the photoactive layer 135 may absorb light to generate an electrical signal. In some embodiments, the organic compound included in the first layer 131 may serve as a donor for supplying electrons, and the electron accepting compound included in the second layer 132 may serve as an acceptor for receiving electrons. Accordingly, the opto-electronic device 31 including the photoactive layer 135 may serve as a photosensor. For example, the opto-electronic device 31 may serve as a fingerprint recognition sensor, which will be described in more detail herein below with reference to FIG. 5.

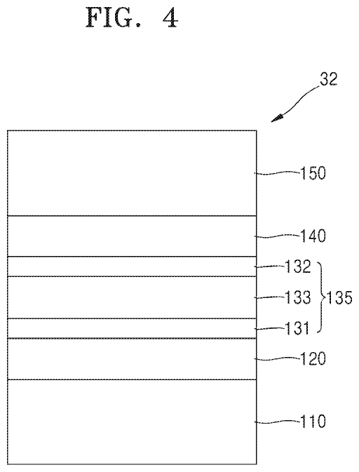

### Description of FIG. 4

FIG. 4 is a schematic view of an opto-electronic device 32 according to one or more embodiments.

The opto-electronic device 32 shown in FIG. 4 is the same as and/or similar to the opto-electronic device 31 shown in FIG. 3, except for the photoactive layer 135, and thus, descriptions of other components are omitted (e.g., are not provided).

The opto-electronic device 32 may include the photoactive layer 135 arranged between the hole transport region 120 and the electron transport region 140. In one or more embodiments, the photoactive layer 135 may be arranged between the hole transport layer included in the hole transport region 120 and the buffer layer included in the electron transport region 140. In one or more embodiments, the photoactive layer 135 may be arranged between the emission auxiliary layer included in the hole transport region 120 and the buffer layer included in the electron transport region 140.

The photoactive layer 135 may include the first layer 131 adjacent to the hole transport region 120, the second layer 132 adjacent to the electron transport region 140, and a third layer 133 arranged between the first layer 131 and the second layer 132. In one or more embodiments, the third layer 133 may be in direct contact with the first layer 131 and/or the second layer 132.

In one or more embodiments, the first layer 131 may be in direct contact with the hole transport layer included in the hole transport region 120. In one or more embodiments, the first layer 131 may be in direct contact with the emission auxiliary layer arranged on the hole transport layer.

In one or more embodiments, the second layer 132 may be in direct contact with the buffer layer included in the electron transport region 140.

The first layer 131 may include the organic compound. For example, the first layer 131 may not include (e.g., may exclude) the electron accepting compound.

The second layer 132 may include the electron accepting compound. For example, the second layer 132 may not include (e.g., may exclude) the organic compound.

The third layer 133 may include the organic compound and the electron accepting compound. For example, the organic compound and the electron accepting compound may be mixed and included in the third layer 133.

For example, the photoactive layer 135 may have a multi-layered structure divided into the first layer 131 including the organic compound, the third layer 133 including both (e.g., simultaneously) the organic compound and the electron accepting compound, and the second layer 132 including the electron accepting compound.

The photoactive layer 135 may absorb incident light to form excitons. The excitons may generate holes and electrons. For example, the photoactive layer 135 may absorb light to generate an electrical signal. In one or more embodiments, the organic compound included in each of the first layer 131 and the third layer 133 may serve as a donor for supplying electrons, and the electron accepting compound included in each of the second layer 132 and the third layer 133 may serve as an acceptor for receiving electrons. Accordingly, the opto-electronic device 32 including the photoactive layer 135 may serve as a photosensor. For example, the opto-electronic device 32 may serve as a fingerprint recognition sensor, which will be described in more detail herein below with reference to FIG. 5.

### Electronic Apparatus

The light-emitting device 10 and the opto-electronic device 30, 31, or 32 may be included in one or more suitable electronic apparatuses. For example, the electronic apparatus may be a light-emitting apparatus, an authentication apparatus, and/or the like.

The electronic apparatus (e.g., a light-emitting apparatus) may further include, in addition to the light-emitting device 10 and the opto-electronic device 30, 31, or 32, i) a color filter, ii) a color conversion layer, or iii) a color filter and a color conversion layer. The color filter and/or the color conversion layer may be arranged in at least one direction in which light emitted from the light-emitting device 10 travels. For example, the light emitted from the light-emitting device 10 may be blue light or white light. More details on the light-emitting device 10 may be as described above. In one or more embodiments, the color conversion layer may include a quantum dot. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel defining layer may be arranged among the subpixel areas to define each of the subpixel areas.

The color filter may further include a plurality of color filter areas and light-shielding patterns arranged among the color filter areas, and the color conversion layer may further include a plurality of color conversion areas and light-shielding patterns arranged among the color conversion areas.

The color filter areas (or the color conversion areas) may include a first area emitting (e.g., configured to emit) first color light, a second area emitting (e.g., configured to emit) second color light, and/or a third area emitting (e.g., configured to emit) third color light, and the first color light, the second color light, and/or the third color light may have different maximum emission wavelengths. For example, the first color light may be red light, the second color light may be green light, and the third color light may be blue light. For example, the color filter areas (or the color conversion areas) may include quantum dots. In one or more embodiments, the first area may include a red quantum dot, the second area may include a green quantum dot, and the third area may not include (e.g., may exclude) a quantum dot. More details on the quantum dot may be as described herein. The first area, the second area, and/or the third area may each further include a scatterer.

For example, the light-emitting device 10 may emit first light, the first area may absorb the first light to emit first-first color light, the second area may absorb the first light to emit second-first color light, and the third area may absorb the first light to emit third-first color light. In this regard, the first-first color light, the second-first color light, and the third-first color light may have different maximum emission wavelengths. In one or more embodiments, the first light may be blue light, the first-first color light may be red light, the second-first color light may be green light, and the third-first color light may be blue light.

The electronic apparatus may further include a thin-film transistor, in addition to the opto-electronic device 30, 31, or 32 and the light-emitting device 10 as described above. The thin-film transistor may include a source electrode, a drain electrode, and an active layer, and the source electrode or the drain electrode may be electrically connected to the first electrode 110 or the second electrode 150 of the light-emitting device 10.

The thin-film transistor may further include a gate electrode, a gate insulating film, and/or the like.

The active layer may include crystalline silicon, amorphous silicon, an organic semiconductor, an oxide semiconductor, and/or the like.

The electronic apparatus may further include a sealing portion for sealing the opto-electronic device 30, 31, or 32 and the light-emitting device 10. The sealing portion may be arranged between the color filter and/or the color conversion layer and the light-emitting device 10. The sealing portion may allow light from the light-emitting device 10 to be extracted to the outside, and may concurrently (e.g., simultaneously) prevent or reduce ambient air and/or moisture from penetrating into the opto-electronic device 30, 31, or 32 and the light-emitting device 10. The sealing portion may be a sealing substrate including a transparent glass substrate and/or a plastic substrate. The sealing portion may be a thin-film encapsulation layer including at least one layer selected from an organic layer and an inorganic layer. When the sealing portion is a thin-film encapsulation layer, the electronic apparatus may be flexible.

One or more suitable functional layers may be additionally arranged on the sealing portion, in addition to the color filter and/or the color conversion layer, according to the desired use of the electronic apparatus. Examples of the functional layer may include a touch screen layer, a polarizing layer, and/or the like. The touch screen layer may be a pressure-sensitive touch screen layer, a capacitive touch screen layer, and/or an infrared touch screen layer. The authentication apparatus may be, for example, a biometric authentication apparatus that authenticates an individual by utilizing biometric information of a living body (e.g., fingertips, pupils, etc.).

The authentication apparatus may further include, in addition to the opto-electronic device 30, 31, or 32 and the light-emitting device 10 as described above, a biometric information collector.

The electronic apparatus may be applied to one or more suitable displays, light sources, lighting, personal computers (e.g., a mobile personal computer), mobile phones, digital cameras, electronic organizers, electronic dictionaries, electronic game machines, medical instruments (e.g., electronic thermometers, sphygmomanometers, blood glucose meters, pulse measurement devices, pulse wave measurement devices, electrocardiogram displays, ultrasonic diagnostic devices, and/or endoscope displays), fish finders, one or more suitable measuring instruments, meters (e.g., meters for a vehicle, an aircraft, and/or a vessel), projectors, and/or the like.

### Electronic Device

The opto-electronic device 30, 31, or 32 may be included in one or more suitable electronic devices.

For example, the electronic device including the opto-electronic device 30, 31, and/or 32 may be one selected from a flat panel display, a curved display, a computer monitor, a medical monitor, a television, a billboard, an indoor and/or outdoor lighting and/or signal light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a three-dimensional (3D) display, a virtual and/or augmented-reality display, a vehicle, a video wall including multiple displays tiled together, a theater and/or stadium screen, a phototherapy device, and a signboard.

Because the opto-electronic device 30, 31, and/or 32 has excellent or suitable photoelectric characteristics, the electronic device including the opto-electronic device 30, 31, and/or 32 may serve as an optical sensor, such as a fingerprint recognition sensor.

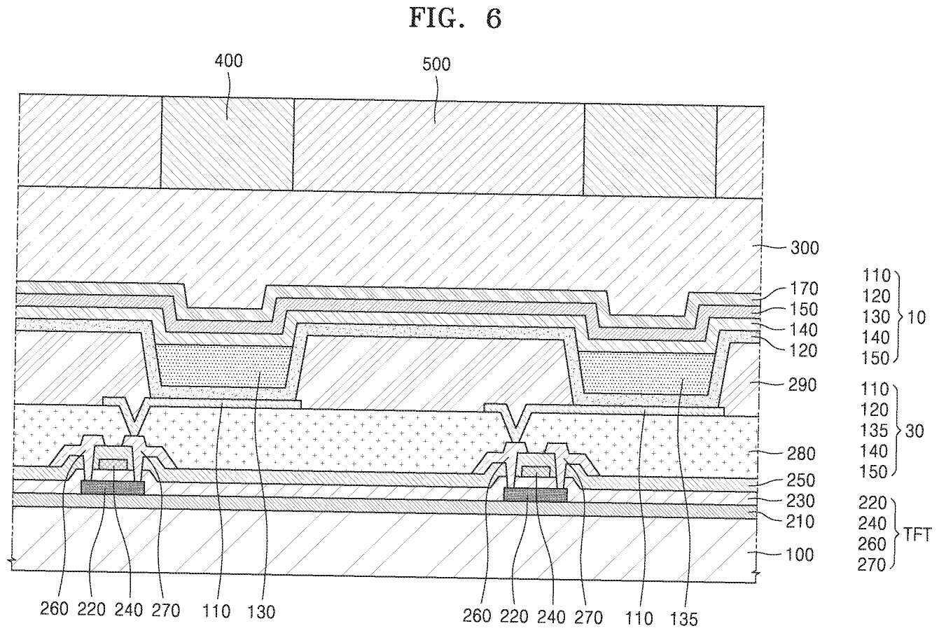

### Description of FIGS. 5 and 6

FIG. 5 is a cross-sectional view of an electronic apparatus according to one or more embodiments.

The electronic apparatus of FIG. 5 may include a substrate 100, a thin-film transistor TFT, a light-emitting device 10, an opto-electronic device 30, and an encapsulation portion 300. The opto-electronic device 30 of FIG. 5 may be the opto-electronic device 30 described above with reference to FIG. 1, but is not limited thereto. For example, the opto-electronic device 30 of FIG. 5 may be the opto-electronic device 31 of FIG. 3 or the opto-electronic device 32 of FIG. 4.

The substrate 100 may be a flexible substrate, a glass substrate, and/or a metal substrate. A buffer layer 210 may be arranged on the substrate 100. The buffer layer 210 may prevent or reduce penetration of impurities through the substrate 100 and may provide a substantially or suitably flat surface on the substrate 100.

The thin-film transistor TFT may be arranged on the buffer layer 210. The thin-film transistor TFT may include an active layer 220, a gate electrode 240, a source electrode 260, and a drain electrode 270.

The active layer 220 may include an inorganic semiconductor, such as silicon and/or polysilicon, an organic semiconductor, and/or an oxide semiconductor, and may include a source region, a drain region, and a channel region.

A gate insulating film 230 for insulating the active layer 220 from the gate electrode 240 may be arranged on the active layer 220, and the gate electrode 240 may be arranged on the gate insulating film 230.

An interlayer insulating film 250 may be arranged on the gate electrode 240. The interlayer insulating film 250 may be arranged between the gate electrode 240 and the source electrode 260 to insulate the gate electrode 240 from the source electrode 260, and between the gate electrode 240 and the drain electrode 270 to insulate the gate electrode 240 from the drain electrode 270.

The source electrode 260 and the drain electrode 270 may be arranged on the interlayer insulating film 250. The interlayer insulating film 250 and the gate insulating film 230 may be formed to expose the source region and the drain region of the active layer 220, and the source electrode 260 and the drain electrode 270 may be in contact with the exposed portions of the source region and the drain region of the active layer 220.

The light-emitting device 10 and the opto-electronic device 30 may be arranged on the thin-film transistor TFT.

The thin-film transistor TFT electrically connected to the light-emitting device 10 may transmit an electrical signal for driving the light-emitting device 10. The thin-film transistor TFT electrically connected to the opto-electronic device 30 may transmit an electrical signal generated by the opto-electronic device 30. The thin-film transistor TFT may be covered and protected by a passivation layer 280. The passivation layer 280 may include an inorganic insulating film, an organic insulating film, or any combination thereof. The light-emitting device 10 and the opto-electronic device 30 may be provided on the passivation layer 280.

The light-emitting device 10 may include a first electrode 110, a hole transport region 120, an emission layer 130, an electron transport region 140, and a second electrode 150. The opto-electronic device 30 may include a first electrode 110, a hole transport region 120, a photoactive layer 135, an electron transport region 140, a second electrode 150.

The first electrode 110 may be arranged on the passivation layer 280. The passivation layer 280 may expose certain regions of the source electrode 260 and the drain electrode 270 without completely covering the source electrode 260 and the drain electrode 270, and the first electrode 110 may be connected to the exposed regions of the source electrode 260 and the drain electrode 270.

A pixel defining layer 290 including an insulating material may be arranged on the first electrode 110. The pixel defining layer 290 may expose a certain region of the first electrode 110. The pixel defining layer 290 may be a polyimide and/or polyacrylic organic film.

The hole transport region 120 may be arranged on the pixel defining layer 290. The hole transport region 120 included in the light-emitting device 10 and the hole transport region 120 included in the opto-electronic device 30 may be integrally formed as a single body. The hole transport region 120 included in the light-emitting device 10 and the hole transport region 120 included in the opto-electronic device 30 may be arranged on the pixel defining layer 290, may be connected to each other (e.g., physically and/or electrically connected), may include substantially the same material, and may be formed substantially at the same time.

Each of the emission layer 130 and the photoactive layer 135 may be arranged on the hole transport region 120. Each of the emission layer 130 and the photoactive layer 135 may overlap the certain region of the first electrode 110 that is exposed by the pixel defining layer 290.

The electron transport region 140 may be arranged on the emission layer 130 and the photoactive layer 135. The electron transport region 140 included in the light-emitting device 10 and the electron transport region 140 included in the opto-electronic device 30 may be integrally formed as a single body. The electron transport region 140 included in the light-emitting device 10 and the electron transport region 140 included in the opto-electronic device 30 may be arranged on the pixel defining layer 290, may be connected to each other (e.g., physically and/or electrically connected), may include substantially the same material, and may be formed substantially at the same time.

The second electrode 150 may be arranged on the electron transport region 140. The second electrode 150 included in the light-emitting device 10 and the second electrode 150 included in the opto-electronic device 30 may be integrally formed as a single body. The second electrode 150 included in the light-emitting device 10 and the second electrode 150 included in the opto-electronic device 30 may be arranged on the pixel defining layer 290, may be connected to each other (e.g., physically and/or electrically connected), may include substantially the same material, and may be formed substantially at the same time.

A capping layer 170 may be additionally formed on the second electrode 150. The capping layer 170 may be formed to cover the second electrode 150.

The encapsulation portion 300 may be arranged on the capping layer 170. The encapsulation portion 300 may be arranged on the light-emitting device 10 and the opto-electronic device 30 to protect the light-emitting device 10 and the opto-electronic device 30 from moisture and/or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (e.g., polymethyl methacrylate, polyacrylic acid, etc.), an epoxy-based resin (e.g., aliphatic glycidyl ether (AGE), etc.), or any combination thereof; or a combination of the inorganic film(s) and/or the organic film(s).

The light-emitting device 10 may emit lights L1, L2, and L3. For example, the lights L1, L2, and L3 may each be green light.

The light L3 from among the lights L1, L2, and L3 that have been emitted may be incident on an object 600 outside the electronic apparatus. For example, the object 600 may be a finger of a user of the electronic apparatus. A light L3′ reflected by the object 600 may be incident on the opto-electronic device 30.

The photoactive layer 135 may absorb the incident light L3′ to form excitons. The excitons may generate holes and electrons. For example, the photoactive layer 135 may absorb light to generate an electrical signal. For example, the organic compound included in the photoactive layer 135 may serve as a donor for supplying electrons, and the electron accepting compound included in the photoactive layer 135 may serve as an acceptor for receiving electrons. The opto-electronic device 30 may detect energy of the light L3′ and convert the detected energy into an electrical signal. Accordingly, the opto-electronic device 30 may recognize the object 600 that has come into contact with (or approached) the electronic apparatus. Accordingly, the opto-electronic device 30 including the photoactive layer 135 may serve as an optical sensor (e.g., a fingerprint recognition sensor).

FIG. 6 is a cross-sectional view of an electronic apparatus according to one or more embodiments.

The electronic apparatus of FIG. 6 is the same as the electronic apparatus of FIG. 5, except that a light shielding pattern 500 and a functional region 400 are additionally arranged on the encapsulation portion 300. The functional region 400 may be i) a color filter area, ii) a color conversion area, or iii) a combination of the color filter area and the color conversion area. In one or more embodiments, the light-emitting device included in the electronic apparatus of FIG. 6 may be a tandem light-emitting device.

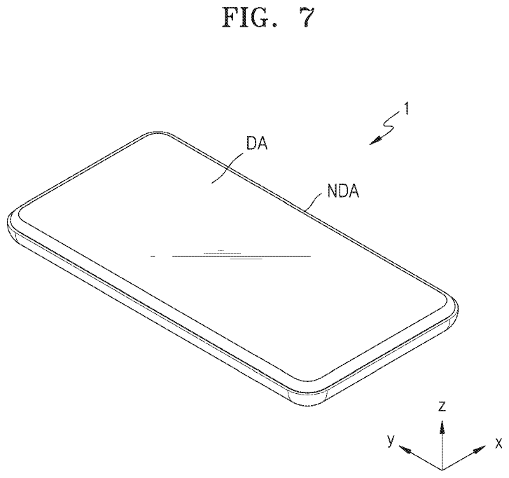

### Description of FIG. 7

FIG. 7 is a schematic perspective view of an electronic device 1 including an opto-electronic device according to one or more embodiments. The electronic device 1 may be an apparatus that displays a moving image and/or a still image, and may include a portable electronic device, such as a mobile phone, a smart phone, a tablet personal computer (PC), a mobile communication terminal, an electronic notebook, an electronic book, a portable multimedia player (PMP), a navigation device, and/or an ultra mobile PC (UMPC), as well as one or more suitable products, such as a television, a laptop, a monitor, a billboard, an internet of things (IOT) device, and/or a part thereof. In some embodiments, the electronic device 1 may be a wearable device, such as a smart watch, a watch phone, a glasses-type or kind display, a head mounted display (HMD), and/or a part thereof. However, embodiments are not limited thereto. For example, the electronic device 1 may be an instrument panel of a vehicle, a center information display (CID) arranged on a center fascia or a dashboard of a vehicle, a room mirror display that replaces a side mirror of a vehicle, an entertainment display for a rear seat of a vehicle and/or a display arranged on a rear surface of a front seat, a head up display (HUD) installed at a front of a vehicle and/or projected on a front window glass, and/or a computer generated hologram augmented reality head up display (CGH AR HUD). FIG. 7 shows a case in which the electronic device 1 is a smart phone, for convenience of description.

The electronic device 1 may include a display area DA and a non-display area NDA outside the display area DA. The electronic device 1 may implement an image through an array of a plurality of pixels that are two-dimensionally arranged in the display area DA.

The non-display area NDA is an area in which an image is not displayed, and may entirely surround the display area DA. A driver for providing electrical signals and/or power to display elements arranged in the display area DA may be arranged in the non-display area NDA. A pad, which is an area to which an electronic element and/or a printed circuit board may be electrically connected, may be arranged in the non-display area NDA.

The electronic device 1 may have different lengths in the x-axis direction and in the y-axis direction. For example, as shown in FIG. 7, the length in the x-axis direction may be shorter than the length in the y-axis direction. In one or more embodiments, the length in the x-axis direction may be the same as the length in the y-axis direction. In one or more embodiments, the length in the x-axis direction may be longer than the length in the y-axis direction.

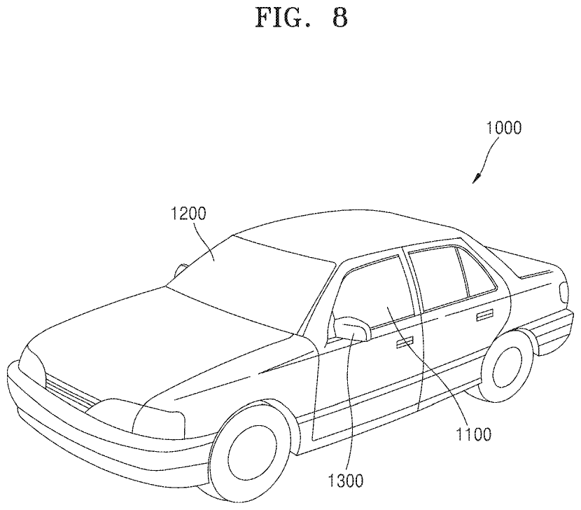

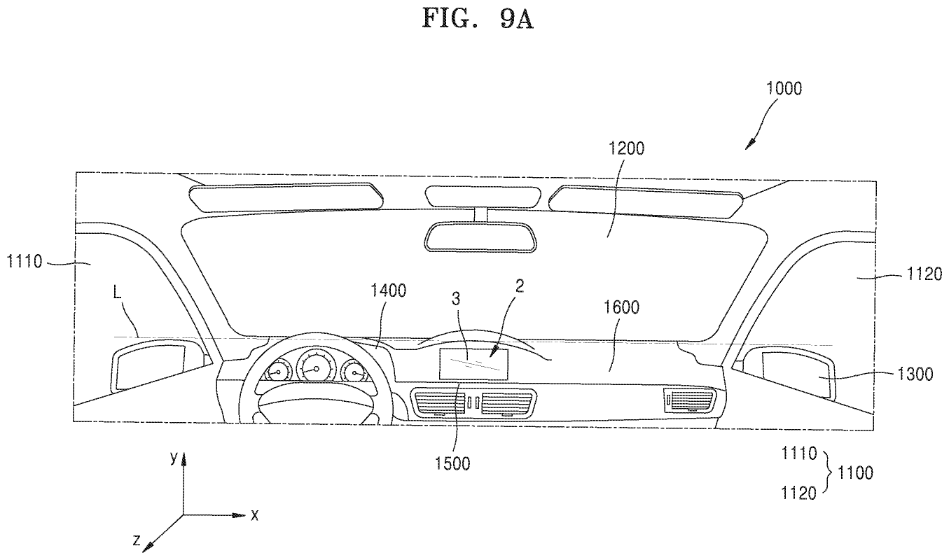

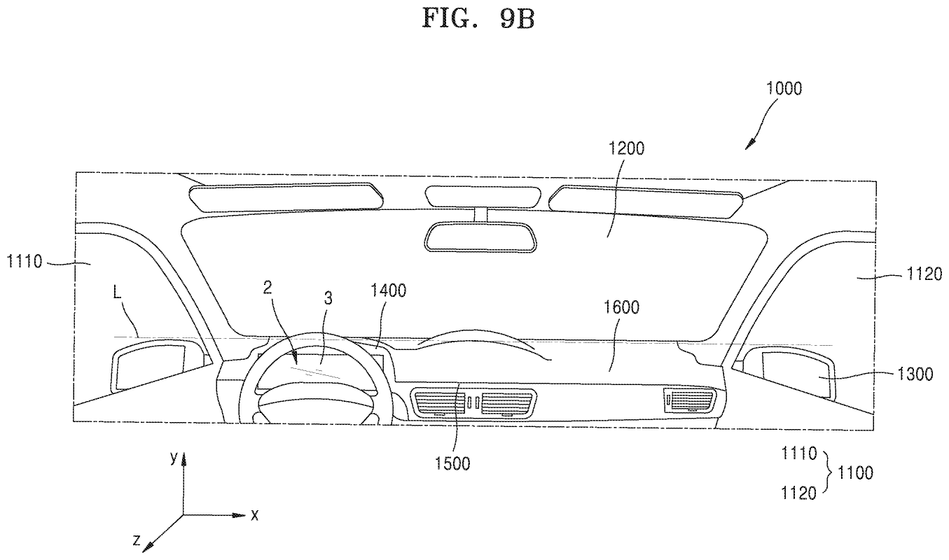

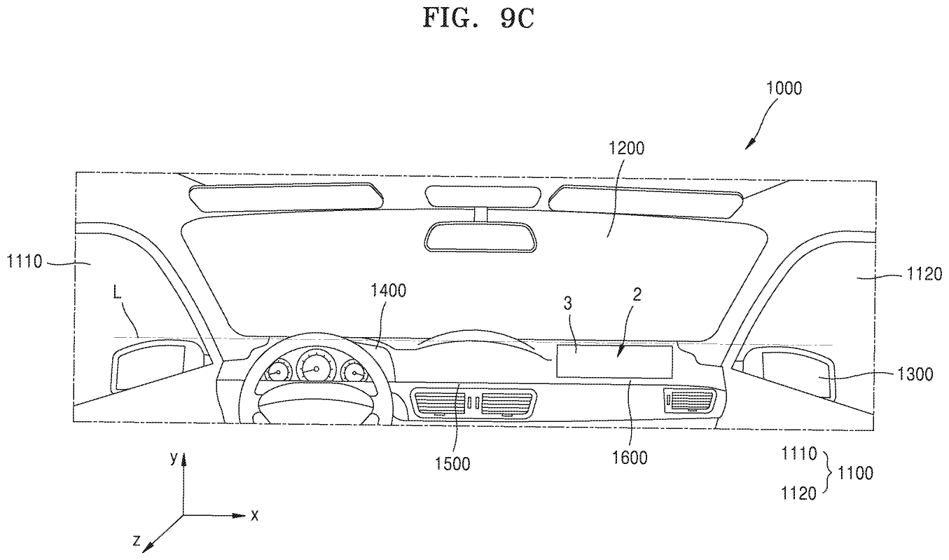

### Description of FIGS. 8 and 9A to 9C

FIG. 8 is a schematic view of the exterior of a vehicle 1000 as an electronic device including an opto-electronic device according to one or more embodiments. FIGS. 9A to 9C are schematic views each being of an interior of the vehicle 1000 according to one or more suitable embodiments.

Referring to FIGS. 8, 9A, 9B, and 9C, the vehicle 1000 may refer to one or more suitable apparatuses for moving a subject to be transported, such as a human, an object, and/or an animal, from a departure point to a destination. The vehicle 1000 may include a vehicle traveling on a road and/or a track, a vessel moving over the sea and/or a river, an airplane flying in the sky by utilizing the action of air, and/or the like.

The vehicle 1000 may travel on a road and/or a track. The vehicle 1000 may move in a certain direction according to rotation of at least one wheel. For example, the vehicle 1000 may include a three-wheeled or four-wheeled vehicle, a construction machine, a two-wheeled vehicle, a prime mover apparatus, a bicycle, and/or a train running on a track.

The vehicle 1000 may include a body having an interior and an exterior, and a chassis in which mechanical apparatuses necessary for driving are installed as the remaining parts except for the body. The exterior of the body may include a front panel, a bonnet, a roof panel, a rear panel, a trunk, and a pillar provided at a boundary between doors. The chassis of the vehicle 1000 may include a power generating apparatus, a power transmitting apparatus, a driving apparatus, a steering apparatus, a braking apparatus, a suspension apparatus, a transmission apparatus, a fuel apparatus, front and rear wheels, left and right wheels, and/or the like.

The vehicle 1000 may include a side window glass 1100, a front window glass 1200, a side mirror 1300, a cluster 1400, a center fascia 1500, a passenger seat dashboard 1600, and a display apparatus 2.

The side window glass 1100 and the front window glass 1200 may be partitioned by a pillar arranged between the side window glass 1100 and the front window glass 1200.

The side window glass 1100 may be installed on the side of the vehicle 1000. In one or more embodiments, the side window glass 1100 may be installed on a door of the vehicle 1000. A plurality of side window glasses 1100 may be provided and may face each other. In one or more embodiments, the side window glass 1100 may include a first side window glass 1110 and a second side window glass 1120. In one or more embodiments, the first side window glass 1110 may be arranged adjacent to the cluster 1400. The second side window glass 1120 may be arranged adjacent to the passenger seat dashboard 1600.

In one or more embodiments, the side window glasses 1100 may be apart (e.g., spaced) from each other in the x-direction and/or the −x-direction. For example, the first side window glass 1110 and the second side window glass 1120 may be apart (e.g., spaced) from each other in the x direction and/or the −x direction. In some embodiments, an imaginary straight line L connecting the side window glasses 1100 to each other may extend in the x direction and/or the −x direction. For example, the imaginary straight line L connecting the first side window glass 1110 and the second side window glass 1120 to each other may extend in the x direction and/or the −x direction.

The front window glass 1200 may be installed in the front of the vehicle 1000. The front window glass 1200 may be arranged between the side window glasses 1100 facing each other.

The side mirror 1300 may provide a view of the rear of the vehicle 1000. The side mirror 1300 may be installed on the exterior of the body. In one or more embodiments, a plurality of side mirrors 1300 may be provided. One of the plurality of side mirrors 1300 may be arranged outside the first side window glass 1110. Another one of the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge, a turn indicator, a high beam indicator, a warning lamp, a seat belt warning lamp, an odometer, a hodometer, an automatic transmission selection lever indicator, a door open warning lamp, an engine oil warning lamp, and/or a low fuel warning lamp.

The center fascia 1500 may include a control panel on which a plurality of buttons for adjusting an audio apparatus, an air conditioning apparatus, and a heater of a seat are arranged. The center fascia 1500 may be arranged on one side of the cluster 1400.

The passenger seat dashboard 1600 may be apart (e.g., spaced) from the cluster 1400 with the center fascia 1500 therebetween. In one or more embodiments, the cluster 1400 may be arranged to correspond to a driver seat, and the passenger seat dashboard 1600 may be arranged to correspond to a passenger seat. In one or more embodiments, the cluster 1400 may be adjacent to the first side window glass 1110, and the passenger seat dashboard 1600 may be adjacent to the second side window glass 1120.

In one or more embodiments, the display apparatus 2 may include a display panel 3, and the display panel 3 may display an image. The display apparatus 2 may be arranged inside the vehicle 1000. In one or more embodiments, the display apparatus 2 may be arranged between the side window glasses 1100 facing each other. The display apparatus 2 may be arranged on at least one selected from the cluster 1400, the center fascia 1500, and the passenger seat dashboard 1600.

The display apparatus 2 may include an organic light-emitting display apparatus, an inorganic electroluminescent (EL) display apparatus, a quantum dot display apparatus, and/or the like. Hereinafter, as the display apparatus 2 according to one or more embodiments, an organic light-emitting display apparatus including the light-emitting device according to one or more embodiments will be described as an example, but one or more suitable types (kinds) of display apparatuses as described above may be utilized in embodiments.

Referring to FIG. 9A, the display apparatus 2 may be arranged on the center fascia 1500. In one or more embodiments, the display apparatus 2 may display navigation information. In one or more embodiments, the display apparatus 2 may display audio, video, and/or information regarding vehicle settings.

Referring to FIG. 9B, the display apparatus 2 may be arranged on the cluster 1400. In this case, the cluster 1400 may display driving information and/or the like through the display apparatus 2. For example, the cluster 1400 may be digitally implemented. The digital cluster 1400 may display vehicle information and/or driving information as images. For example, a needle and a gauge of a tachometer and one or more suitable warning lamp icons may be displayed by digital signals.

Referring to FIG. 9C, the display apparatus 2 may be arranged on the passenger seat dashboard 1600. The display apparatus 2 may be embedded in the passenger seat dashboard 1600 or arranged on the passenger seat dashboard 1600. In one or more embodiments, the display apparatus 2 arranged on the passenger seat dashboard 1600 may display an image related to information displayed on the cluster 1400 and/or information displayed on the center fascia 1500. In one or more embodiments, the display apparatus 2 arranged on the passenger seat dashboard 1600 may display information that is different from information displayed on the cluster 1400 and/or information displayed on the center fascia 1500.

### Manufacturing Method

Respective layers included in the hole transport region 120, the emission layer 130, the photoactive layer 135, and respective layers included in the electron transport region 140 may be formed in a certain region by utilizing one or more suitable methods selected from vacuum deposition, spin coating, casting, Langmuir-Blodgett (LB) deposition, ink-jet printing, laser-printing, laser-induced thermal imaging, and/or the like.

When respective layers included in the hole transport region 120, the emission layer 130, the photoactive layer 135, and respective layers included in the electron transport region 140 are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group” as utilized herein refers to a cyclic group that has 3 to 60 carbon atoms and includes (e.g., consists of) only carbon atoms as ring-forming atoms. The term “C₁-C₆₀ heterocyclic group” as utilized herein refers to a cyclic group that has 1 to 60 carbon atoms and further includes, in addition to carbon atoms, at least one heteroatom as a ring-forming atom. The C₃-C₆₀ carbocyclic group and the C₁-C₆₀ heterocyclic group may each be a monocyclic group including (e.g., consisting of) one ring or a polycyclic group in which two or more rings are condensed with each other. For example, the C₁-C₆₀ heterocyclic group may have 3 to 61 ring-forming atoms.

The term “cyclic group” as utilized herein may include both the C₃-C₆₀ carbocyclic group and the C₁-C₆₀ heterocyclic group.

The term “π electron-rich C₃-C₆₀ cyclic group” as utilized herein refers to a cyclic group that has 3 to 60 carbon atoms and does not include *—N═*′ as a ring-forming moiety, where * and *′ are binding sites to corresponding neighboring atoms.

The term “π electron-deficient nitrogen-containing C₁-C₆₀ cyclic group” as utilized herein refers to a heterocyclic group that has 1 to 60 carbon atoms and includes *—N═*′ as a ring-forming moiety, where * and *′ are binding sites to corresponding neighboring atoms.

For example,
the C₃-C₆₀ carbocyclic group may be i) a group T1 or ii) a condensed cyclic group in which two or more groups T1 are condensed with each other (e.g., the C₃-C₆₀ carbocyclic group may be a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) a group T2, ii) a condensed cyclic group in which two or more groups T2 are condensed with each other, or iii) a condensed cyclic group in which at least one group T2 and at least one group T1 are condensed with each other (e.g., the C₁-C₆₀ heterocyclic group may be a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
the π electron-rich C₃-C₆₀ cyclic group may be i) a group T1, ii) a condensed cyclic group in which two or more groups T1 are condensed with each other, iii) a group T3, iv) a condensed cyclic group in which two or more groups T3 are condensed with each other, or v) a condensed cyclic group in which at least one group T3 and at least one group T1 are condensed with each other (e.g., the π electron-rich C₃-C₆₀ cyclic group may be the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.), and
the π electron-deficient nitrogen-containing C₁-C₆₀ cyclic group may be i) a group T4, ii) a condensed cyclic group in which two or more groups T4 are condensed with each other, iii) a condensed cyclic group in which at least one group T4 and at least one group T1 are condensed with each other, iv) a condensed cyclic group in which at least one group T4 and at least one group T3 are condensed with each other, or v) a condensed cyclic group in which at least one group T4, at least one group T1, and at least one group T3 are condensed with each other (e.g., the π electron-deficient nitrogen-containing C₁-C₆₀ cyclic group may be a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.).

The group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group.

The group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group.

The group T3 may be a furan group, a thiophene group, a 1H-pyrrole group, a silole group, or a borole group.

The group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The term “cyclic group,” “C₃-C₆₀ carbocyclic group,” “C₁-C₆₀ heterocyclic group,” “π electron-rich C₃-C₆₀ cyclic group,” and/or “π electron-deficient nitrogen-containing C₁-C₆₀ cyclic group” as utilized herein refers to a group condensed with any suitable cyclic group, a monovalent group, or a polyvalent group (e.g., a divalent group, a trivalent group, a tetravalent group, etc.), depending on the structure of a formula in connection with which the terms are utilized.

For example, the “benzene group” may be a benzo group, a phenyl group, a phenylene group, and/or the like, which may be easily understood by one of ordinary skill in the art according to the structure of a formula including the “benzene group.”

Examples of the monovalent C₃-C₆₀ carbocyclic group and the monovalent C₁-C₆₀ heterocyclic group may include a C₃-C₁ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

Examples of the polyvalent (e.g., divalent) C₃-C₆₀ carbocyclic group and the polyvalent (e.g., divalent) C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a polyvalent (e.g., divalent) non-aromatic condensed polycyclic group, and a polyvalent (e.g., divalent) non-aromatic condensed heteropolycyclic group.

The term “C₁-C₆₀ alkyl group” as utilized herein refers to a linear or branched aliphatic hydrocarbon monovalent group that has 1 to 60 carbon atoms, and examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, and/or the like.

The term “C₁-C₆₀ alkylene group” as utilized herein refers to a polyvalent (e.g., divalent) group having the same structure as the C₁-C₆₀ alkyl group.

The term “C₂-C₆₀ alkenyl group” as utilized herein refers to a monovalent hydrocarbon group having at least one carbon-carbon double bond in the middle and/or at the terminus of the C₂-C₆₀ alkyl group, and examples thereof may include an ethenyl group, a propenyl group, a butenyl group, and/or the like.

The term “C₂-C₆₀ alkenylene group” as utilized herein refers to a polyvalent (e.g., divalent) group having the same structure as the C₂-C₆₀ alkenyl group.

The term “C₂-C₆₀ alkynyl group” as utilized herein refers to a monovalent hydrocarbon group having at least one carbon-carbon triple bond in the middle and/or at the terminus of the C₂-C₆₀ alkyl group, and examples thereof may include an ethynyl group, a propynyl group, and/or the like.

The term “C₂-C₆₀ alkynylene group” as utilized herein refers to a polyvalent (e.g., divalent) group having the same structure as the C₂-C₆₀ alkynyl group.

The term “C₁-C₆₀ alkoxy group” as utilized herein refers to a monovalent group represented by —OA₁₀₁ (wherein A₁₀₁ is the C₁-C₆₀ alkyl group), and examples thereof may include a methoxy group, an ethoxy group, an isopropyloxy group, and/or the like.

The term “C₃-C₁₀ cycloalkyl group” as utilized herein refers to a monovalent saturated hydrocarbon cyclic group having 3 to 10 carbon atoms, and examples thereof may include a cyclopropyl group, a cyclobutyl group, a cyclopentyl group, a cyclohexyl group, a cycloheptyl group, a cyclooctyl group, an adamantanyl group, a norbornanyl group (or a bicyclo[2.2.1]heptyl group), a bicyclo[1.1.1]pentyl group, a bicyclo[2.1.1]hexyl group, a bicyclo[2.2.2]octyl group, and/or the like.

The term “C₃-C₁₀ cycloalkylene group” as utilized herein refers to a polyvalent (e.g., divalent) group having the same structure as the C₃-C₁₀ cycloalkyl group.

The term “C₁-C₁₀ heterocycloalkyl group” as utilized herein refers to a monovalent cyclic group that has 1 to 10 carbon atoms and further includes, in addition to carbon atoms, at least one heteroatom as a ring-forming atom, and examples thereof may include a 1,2,3,4-oxatriazolidinyl group, a tetrahydrofuranyl group, a tetrahydrothiophenyl group, and/or the like.

The term “C₁-C₁₀ heterocycloalkylene group” as utilized herein refers to a polyvalent (e.g., divalent) group having the same structure as the C₁-C₁₀ heterocycloalkyl group.

The term “C₃-C₁₀ cycloalkenyl group” as utilized herein refers to a monovalent cyclic group that has 3 to 10 carbon atoms and at least one carbon-carbon double bond in the ring thereof and no aromaticity when its molecular structure is considered as a whole, and examples thereof may include a cyclopentenyl group, a cyclohexenyl group, a cycloheptenyl group, and/or the like.

The term “C₃-C₁₀ cycloalkenylene group” as utilized herein refers to a polyvalent (e.g., divalent) group having the same structure as the C₃-C₁₀ cycloalkenyl group.

The term “C₁-C₁₀ heterocycloalkenyl group” as utilized herein refers to a monovalent cyclic group that has 1 to 10 carbon atoms, at least one heteroatom as a ring-forming atom, in addition to carbon atoms, and at least one double bond in the ring thereof. Examples of the C₁-C₁₀ heterocycloalkenyl group may include a 4,5-dihydro-1,2,3,4-oxatriazolyl group, a 2,3-dihydrofuranyl group, a 2,3-dihydrothiophenyl group, and/or the like.

The term “C₁-C₁₀ heterocycloalkenylene group” as utilized herein refers to a polyvalent (e.g., divalent) group having the same structure as the C₁-C₁₀ heterocycloalkenyl group.

The term “C₆-C₆₀ aryl group” as utilized herein refers to a monovalent group having a carbocyclic aromatic system of 6 to 60 carbon atoms.

The term “C₆-C₆₀ arylene group” as utilized herein refers to a polyvalent (e.g., divalent) group having the same structure as the C₆-C₆₀ aryl group.

Examples of the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and/or the like.

When the C₆-C₆₀ aryl group and/or the C₆-C₆₀ arylene group each include two or more rings, the respective two or more rings may be condensed with each other.

The term “C₁-C₆₀ heteroaryl group” as utilized herein refers to a monovalent group that has a heterocyclic aromatic system of 1 to 60 carbon atoms and further includes, in addition to carbon atoms, at least one heteroatom as a ring-forming atom.

The term “C₁-C₆₀ heteroarylene group” as utilized herein refers to a polyvalent (e.g., divalent group) having the same structure as the C₁-C₆₀ heteroaryl group.

Examples of the C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, and/or the like.

When the C₁-C₆₀ heteroaryl group and/or the C₁-C₆₀ heteroarylene group each include respective two or more rings, the two or more rings may be condensed with each other.

The term “monovalent non-aromatic condensed polycyclic group” as utilized herein refers to a monovalent group that has two or more rings condensed with each other, only carbon atoms (e.g., 8 to 60 carbon atoms) as ring-forming atoms, and no aromaticity when its entire molecular structure is considered as a whole. Examples of the monovalent non-aromatic condensed polycyclic group may include an indenyl group, a fluorenyl group, a spiro-bifluorenyl group, a benzofluorenyl group, an indenophenanthrenyl group, an indenoanthracenyl group, and/or the like.

The term “divalent non-aromatic condensed polycyclic group” as utilized herein refers to a polyvalent (e.g., divalent) group having the same structure as the monovalent non-aromatic condensed polycyclic group.

The term “monovalent non-aromatic condensed heteropolycyclic group” as utilized herein refers to a monovalent group that has two or more rings condensed with each other, at least one heteroatom other than carbon atoms (e.g., 1 to 60 carbon atoms), as a ring-forming atom, and no aromaticity when its entire molecular structure is considered as a whole. Examples of the monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphtho silolyl group, a benzofurodibenzofuranyl group, a benzofurodibenzothiophenyl group, a benzothienodibenzothiophenyl group, and/or the like.

The term “divalent non-aromatic condensed heteropolycyclic group” as utilized herein refers to a polyvalent (e.g., divalent) group having the same structure as the monovalent non-aromatic condensed heteropolycyclic group.

The term “C₆-C₆₀ aryloxy group” as utilized herein refers to a group represented by —OA₁₀₂ (wherein A102 is the C₆-C₆₀ aryl group).

The term “C₆-C₆₀ arylthio group” as utilized herein refers to a group represented by —SA₁₀₃ (wherein A₁₀₃ is the C₆-C₆₀ aryl group).

The term “C₇-C₆₀ arylalkyl group” as utilized herein refers to a group represented by -A₁₀₄A₁₀₅ (wherein A₁₀₄ is a C₁-C₅₄ alkylene group, and A₁₀₅ is a C₆-C₅₉ aryl group).

The term “C₂-C₆₀ heteroarylalkyl group” as utilized herein refers to a group represented by -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as utilized herein refers to:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃ and Q₃₁ to Q₃₃ utilized herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “heteroatom” as utilized herein refers to any atom other than a carbon atom. Examples of the heteroatom may include O, S, N, P, Si, B, Ge, Se, or any combination thereof.

The term “third-row transition metal” as utilized herein may include hafnium (Hf), tantalum (Ta), tungsten (W), rhenium (Re), osmium (Os), iridium (Ir), platinum (Pt), gold (Au), and/or the like.

The term “Ph” as utilized herein refers to a phenyl group, the term “Me” as utilized herein refers to a methyl group, the term “Et” as utilized herein refers to an ethyl group, the term “tert-Bu” or “Bu^{t}” as utilized herein refers to a tert-butyl group, and the term “OMe” as utilized herein refers to a methoxy group.

The term “biphenyl group” as utilized herein refers to “a phenyl group substituted with a phenyl group.” For example, the “biphenyl group” may be a substituted phenyl group having a C₆-C₆₀ aryl group as a substituent.

The term “terphenyl group” as utilized herein refers to “a phenyl group substituted with a biphenyl group.” For example, the “terphenyl group” may be a substituted phenyl group having, as a substituent, a C₆-C₆₀ aryl group substituted with a C₆-C₆₀ aryl group.

* and *′ as utilized herein, unless defined otherwise, each refer to a binding site to a corresponding neighboring atom in a corresponding formula or moiety.

The x-axis, y-axis, and z-axis as utilized herein are not limited to three axes in an orthogonal coordinate system, and may be interpreted in a broad sense including these axes. For example, the x-axis, y-axis, and z-axis may be orthogonal to each other, but the x-axis, y-axis, and z-axis may also refer to different directions that are not orthogonal to each other.

Hereinafter, compounds according to embodiments and light-emitting devices according to embodiments will be described in more detail with reference to Synthesis Examples and Examples. The wording “B was utilized instead of A” utilized in describing Synthesis Examples refers to an identical molar equivalent of B being utilized in place of A.

### Synthesis Example 1 (Synthesis of Compound 2)

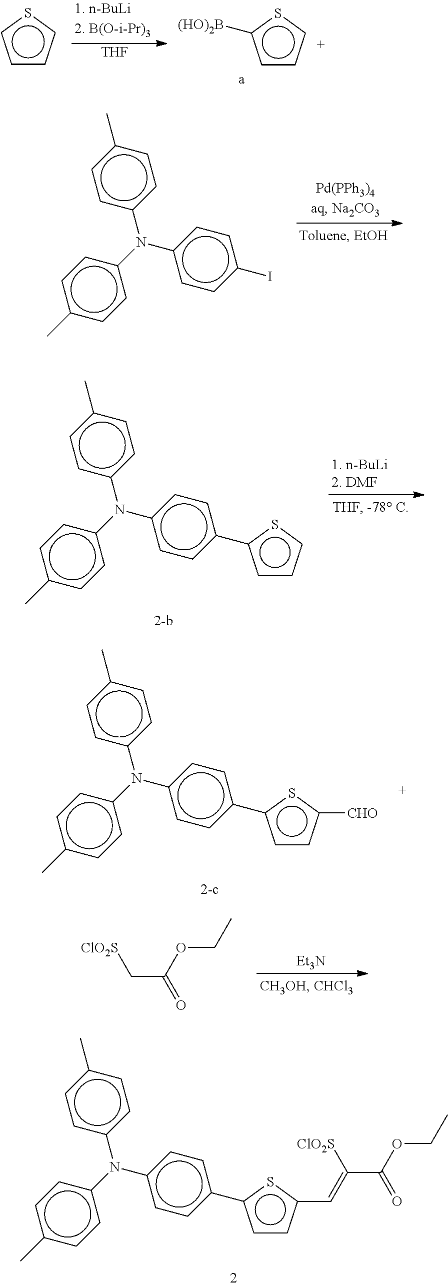

### Synthesis of Intermediate a

In an argon atmosphere, thiophene (2.2 mL, 40 mmol) was dissolved in 40 mL of tetrahydrofuran (THF) and cooled to −78° C. n-BuLi (27.5 mL, 1.6 M in hexanes, 44 mmol) was added dropwise thereto for 10 minutes, and the mixture was stirred for 40 minutes. Then, the mixture was added dropwise to 20 mL of a THF solution of B(O-i-Pr)₃ (13.9 mL, 60 mmol), which had been cooled to 0° C., for 15 minutes. The mixture was stirred at the same temperature for 1 hour and then at room temperature for 17 hours. After completion of the reaction, the solvent was removed therefrom under reduced pressure, and the reaction product was separated and purified by column chromatography utilizing ethyl acetate and hexane to obtain Intermediate a (white solid, 3.8 g, 74%).

By ESI-LCMS, the compound thus obtained was identified as Intermediate a. (ESI-LCMS: [M]⁺: C₄H₅BO₂S. 128.01.)

### Synthesis of Intermediate 2-b

In an argon atmosphere, 4 mL of an ethanol solution of Intermediate a (1.0 g, 7.9 mmol) and 3.6 mL of a 2 M aqueous sodium carbonate solution were added to 10 mL of a toluene solution of 4-iodo-N,N-di-p-tolyamine (1.6 g, 4.0 mmol), and the mixture was purged with nitrogen while being degassed. Next, Pd(PPh₃)₄ (0.3 g, 0.3 mmol) was added thereto, and the mixture was stirred at 80° C. for 12 hours. After cooling, an extraction process was performed thereon utilizing water (500 mL) and ethyl acetate (300 mL) to collect an organic layer, which was then dried utilizing MgSO₄ and filtered. The filtered solution was placed under reduced pressure to remove the solvent therefrom, and the obtained solid was separated and purified by column chromatography utilizing CH₂Cl₂ and hexane to obtain Intermediate 2-b (0.9 g, 2.7 mmol, 66%).

By ESI-LCMS, the compound thus obtained was identified as Intermediate 2-b. (ESI-LCMS: [M]⁺: C₂₄H₂₁NS. 355.14.)

### Synthesis of Intermediate 2-c

In an argon atmosphere, Intermediate 2-b (0.8 g, 2.5 mmol) was dissolved in 50 mL of THF and cooled to −78° C. n-BuLi (1.9 mL, 1.6 M in hexanes, 3.0 mmol) was added thereto, and the mixture was stirred for 30 minutes. Then, dimethylformamide (DMF) (0.2 mL, 3.0 mmol) was added thereto, and the mixture was stirred at the same temperature for 30 minutes and then at room temperature for 10 hours. After cooling, an extraction process was performed thereon utilizing water (500 mL) and ethyl acetate (300 mL) to collect an organic layer, which was then dried utilizing MgSO₄ and filtered. The filtered solution was placed under reduced pressure to remove the solvent therefrom, and the obtained solid was separated and purified by column chromatography utilizing CH₂Cl₂ and hexane to obtain Intermediate 2-c (0.6 g, 1.5 mmol, 62%).

By ESI-LCMS, the compound thus obtained was identified as Intermediate 2-c. (ESI-LCMS: [M]⁺: C₂₅H₂₁NOS. 383.13.)

### Synthesis of Compound 2

In an argon atmosphere, Intermediate 2-c (0.15 g, 0.4 mmol) was dissolved in 5 mL of chloroform, ethyl 2-(chlorosulfonyl)acetate (0.09 g, 0.5 mmol), 5 mL of a methanol solution, and 1 mL of Et₃N were added thereto, and the mixture was stirred at room temperature for 35 minutes and then concentrated under reduced pressure. The obtained solid was separated and purified by column chromatography utilizing CH₂Cl₂ and hexane to obtain Compound 2 (0.16 g, 0.3 mmol, 59%).

By ESI-LCMS, the compound thus obtained was identified as Compound 2. (ESI-LCMS: [M]⁺: C₂₉H₂₆ClNO₄S₂. 551.10.)

### Synthesis Example 2 (Synthesis of Compound 3)

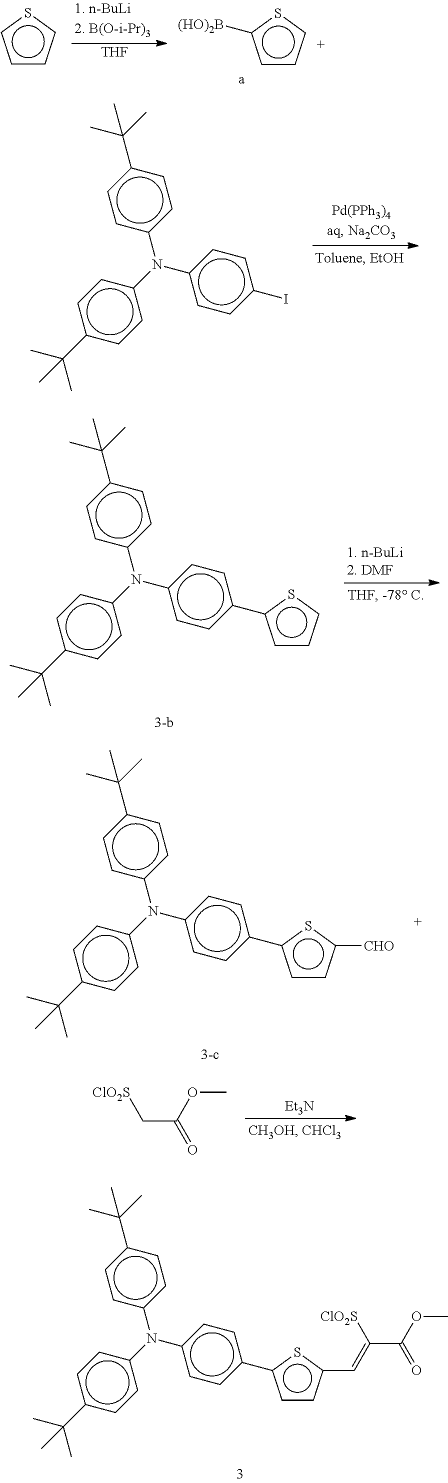

### Synthesis of Intermediate 3-b

Intermediate 3-b (1.1 g, 2.6 mmol, 64%) was obtained in substantially the same manner as utilized to synthesize Intermediate 2-b, except that 4-(tert-butyl)-N-(4-(tert-butyl)phenyl)-N-(4-iodophenyl)aniline (1.9 g, 4.0 mmol) was utilized instead of 4-iodo-N,N-di-p-tolyamine (1.6 g, 4.0 mmol).

By ESI-LCMS, the compound thus obtained was identified as Intermediate 3-b. (ESI-LCMS: [M]⁺: C₃₀H₃₃NS. 439.23.)

### Synthesis of Intermediate 3-c

Intermediate 3-c (0.6 g, 1.4 mmol, 59%) was obtained in substantially the same manner as utilized to synthesize Intermediate 2-c, except that Intermediate 3-b (1.0 g, 2.5 mmol) was utilized instead of Intermediate 2-b (0.8 g, 2.5 mmol).

By ESI-LCMS, the compound thus obtained was identified as Intermediate 3-c. (ESI-LCMS: [M]⁺: C₃₁H₃₃NOS. 467.23.)

### Synthesis of Compound 3

In an argon atmosphere, Intermediate 3-c (0.19 g, 0.4 mmol) was dissolved in 5 mL of chloroform, methyl 2-(chlorosulfonyl)acetate (0.08 g, 0.5 mmol), 5 mL of a methanol solution, and 1 mL of Et₃N were added thereto, and the mixture was stirred at room temperature for 35 minutes and then concentrated under reduced pressure. The obtained solid was separated and purified by column chromatography utilizing CH₂Cl₂ and hexane to obtain Compound 3 (0.19 g, 0.3 mmol, 61%).

By ESI-LCMS, the compound thus obtained was identified as Compound 3. (ESI-LCMS: [M]⁺: C₃₄H₃₆ClNO₄S₂. 621.18.)

### Synthesis Example 3 (Synthesis of Compound 15)

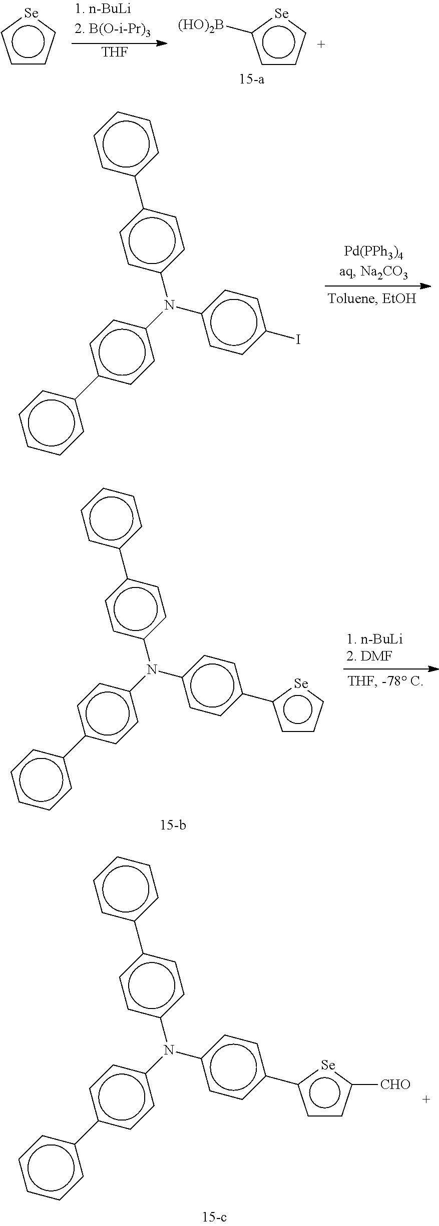

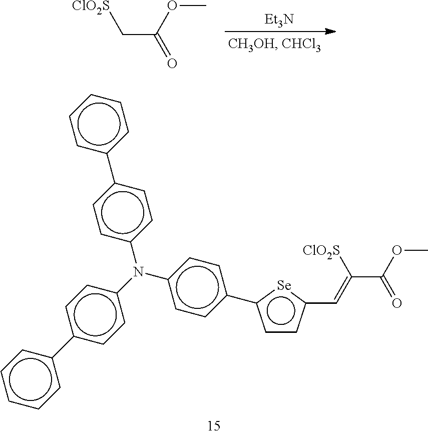

### Synthesis of Intermediate 15-a

In an argon atmosphere, selenophene (5.3 mL, 40 mmol) was dissolved in 40 mL of THF and cooled to −78° C. n-BuLi (27.5 mL, 1.6 M in hexanes, 44 mmol) was added dropwise thereto for 10 minutes, and the mixture was stirred for 40 minutes. Then, the mixture was added dropwise to 20 mL of a THF solution of B(O-i-Pr)₃ (13.9 mL, 60 mmol), which had been cooled to 0° C., for 15 minutes. The mixture was stirred at the same temperature for 1 hour and then at room temperature for 17 hours. After completion of the reaction, the solvent was removed therefrom under reduced pressure, and the reaction product was separated and purified by column chromatography utilizing ethyl acetate and hexane to obtain Intermediate 15-a (white solid, 5.1 g, 73%).

By ESI-LCMS, the compound thus obtained was identified as Intermediate 15-a. (ESI-LCMS: [M]⁺: C₄H₅BO₂Se. 175.95.)

### Synthesis of Intermediate 15-b

In an argon atmosphere, 4 mL of an ethanol solution of Intermediate 15-a (1.4 g, 7.9 mmol) and 3.6 mL of a 2 M aqueous sodium carbonate solution were added to 10 mL of a toluene solution of N-([1,1′-biphenyl]4-yl)-N-(4-iodophenyl)-[1,1′-biphenyl]-4-amine (2.1 g, 4.0 mmol), and the mixture was purged with nitrogen while being degassed. Next, Pd(PPh₃)₄ (0.3 g, 0.3 mmol) was added thereto, and the mixture was stirred at 80° C. for 12 hours. After cooling, an extraction process was performed thereon utilizing water (500 mL) and ethyl acetate (300 mL) to collect an organic layer, which was then dried utilizing MgSO₄ and filtered. The filtered solution was placed under reduced pressure to remove the solvent therefrom, and the obtained solid was separated and purified by column chromatography utilizing CH₂Cl₂ and hexane to obtain Intermediate 15-b (1.4 g, 2.7 mmol, 66%).

By ESI-LCMS, the compound thus obtained was identified as Intermediate 15-b. (ESI-LCMS: [M]⁺: C₃₄H₂₅NSe. 527.12.)

### Synthesis of Intermediate 15-c

In an argon atmosphere, Intermediate 15-b (1.3 g, 2.5 mmol) was dissolved in 50 mL of THF and cooled to −78° C. n-BuLi (1.9 mL, 1.6 M in hexanes, 3.0 mmol) was added thereto, and the mixture was stirred for 30 minutes. Then, DMF (0.2 mL, 3.0 mmol) was added thereto, and the mixture was stirred at the same temperature for 30 minutes and then at room temperature for 10 hours. After cooling, an extraction process was performed thereon utilizing water (500 mL) and ethyl acetate (300 mL) to collect an organic layer, which was then dried utilizing MgSO₄ and filtered. The filtered solution was placed under reduced pressure to remove the solvent therefrom, and the obtained solid was separated and purified by column chromatography utilizing CH₂Cl₂ and hexane to obtain Intermediate 15-c (0.8 g, 1.5 mmol, 62%).

By ESI-LCMS, the compound thus obtained was identified as Intermediate 15-c. (ESI-LCMS: [M]⁺: C₃₅H₂₅NOSe. 555.11.)

### Synthesis of Compound 15

In an argon atmosphere, Intermediate 15-c (0.2 g, 0.4 mmol) was dissolved in 5 mL of chloroform, methyl 2-(chlorosulfonyl)acetate (0.08 g, 0.5 mmol), 5 mL of a methanol solution, and 1 mL of Et₃N were added thereto, and the mixture was stirred at room temperature for 35 minutes and then concentrated under reduced pressure. The obtained solid was separated and purified by column chromatography utilizing CH₂Cl₂ and hexane to obtain Compound 15 (0.1 g, 0.3 mmol, 58%).

By ESI-LCMS, the compound thus obtained was identified as Compound 15. (ESI-LCMS: [M]⁺: C₃₈H₂₈ClNO₄SSe. 709.06.)

### Synthesis Example 4 (Synthesis of Compound 23)

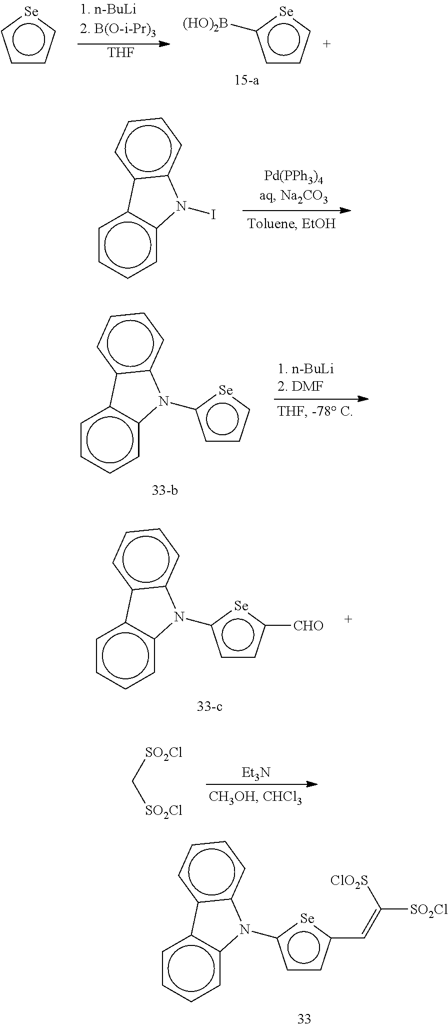

### Synthesis of Intermediate 33-b

Intermediate 33-b (0.8 g, 2.7 mmol, 67%) was obtained in substantially the same manner as utilized to synthesize Intermediate 15-b, except that 9-iodo-9H-carbazole (1.2 g, 4.0 mmol) was utilized instead of N-([1,1′-biphenyl]4-yl)-N-(4-iodophenyl)-[1,1′-biphenyl]-4-amine (2.1 g, 4.0 mmol).

By ESI-LCMS, the compound thus obtained was identified as Intermediate 33-b. (ESI-LCMS: [M]⁺: C₁₆H₁₁NSe. 297.01.)

### Synthesis of Intermediate 33-c

Intermediate 33-c (0.5 g, 1.5 mmol, 65%) was obtained in substantially the same manner as utilized to synthesize Intermediate 15-c, except that Intermediate 33-b (0.7 g, 2.5 mmol) was utilized instead of Intermediate 15-b (1.3 g, 2.5 mmol).

By ESI-LCMS, the compound thus obtained was identified as Intermediate 33-c. (ESI-LCMS: [M]⁺: C₁₇H₁₁NOSe. 325.00.)

### Synthesis of Compound 33

In an argon atmosphere, Intermediate 33-c (0.1 g, 0.4 mmol) was dissolved in 5 mL of chloroform, methanedisulfonyl dichloride (0.1 g, 0.5 mmol), 5 mL of a methanol solution, and 1 mL of Et₃N were added thereto, and the mixture was stirred at room temperature for 35 minutes and then concentrated under reduced pressure. The obtained solid was separated and purified by column chromatography utilizing CH₂Cl₂ and hexane to obtain Compound 33 (0.16 g, 0.3 mmol, 59%).

By ESI-LCMS, the compound thus obtained was identified as Compound 33. (ESI-LCMS: [M]⁺: C₁₈H₁₁ClNO₄S₂Se. 518.87.)

### Synthesis Example 5 (Synthesis of Compound 35)

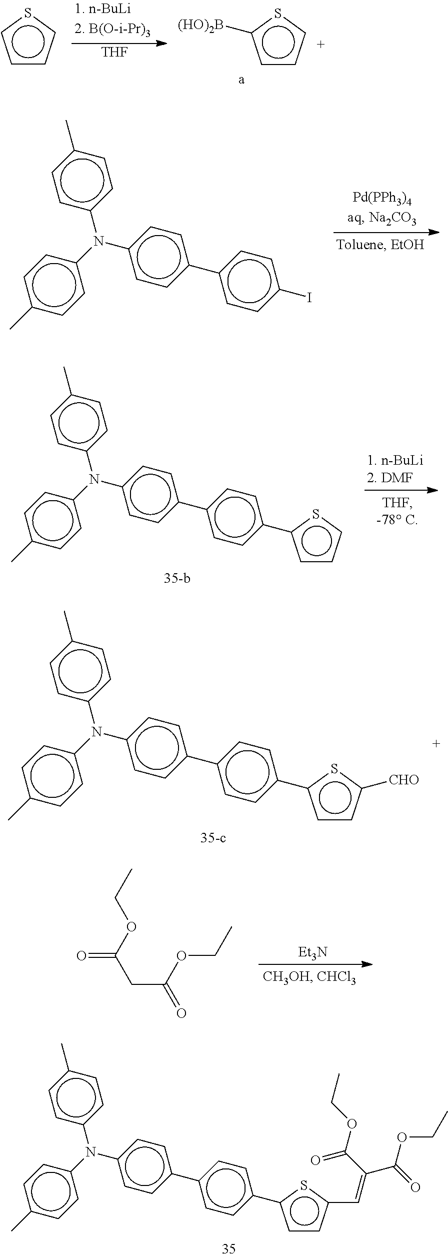

### Synthesis of Intermediate 35-b

In an argon atmosphere, 4 mL of an ethanol solution of Intermediate a (1.0 g, 7.9 mmol) and 3.6 mL of a 2 M aqueous sodium carbonate solution were added to 10 mL of a toluene solution of 4-iodo-N—N-di-p-tolyl-[1,1′-biphenyl]-4-amine, and the mixture was purged with nitrogen while being degassed. Next, Pd(PPh₃)₄ (0.3 g, 0.3 mmol) was added thereto, and the mixture was stirred at 80° C. for 12 hours. After cooling, an extraction process was performed thereon utilizing water (500 mL) and ethyl acetate (300 mL) to collect an organic layer, which was then dried utilizing MgSO₄ and filtered. The filtered solution was placed under reduced pressure to remove the solvent therefrom, and the obtained solid was separated and purified by column chromatography utilizing CH₂Cl₂ and hexane to obtain Intermediate 35-b (1.2 g, 2.7 mmol, 68%).

By ESI-LCMS, the compound thus obtained was identified as Intermediate 35-b. (ESI-LCMS: [M]⁺: C₃₀H₂₅NS. 431.17.)

### Synthesis of Intermediate 35-c

In an argon atmosphere, Intermediate 35-b (1.1 g, 2.5 mmol) was dissolved in 50 mL of THF and cooled to −78° C. n-BuLi (1.9 mL, 1.6 M in hexanes, 3.0 mmol) was added thereto, and the mixture was stirred for 30 minutes. Then, DMF (0.2 mL, 3.0 mmol) was added thereto, and the mixture was stirred at the same temperature for 30 minutes and then at room temperature for 10 hours. After cooling, an extraction process was performed thereon utilizing water (500 mL) and ethyl acetate (300 mL) to collect an organic layer, which was then dried utilizing MgSO₄ and filtered. The filtered solution was placed under reduced pressure to remove the solvent therefrom, and the obtained solid was separated and purified by column chromatography utilizing CH₂Cl₂ and hexane to obtain Intermediate 35-c (0.7 g, 1.5 mmol, 65%).

By ESI-LCMS, the compound thus obtained was identified as Intermediate 35-c. (ESI-LCMS: [M]⁺: C₃₁H₂₅NOS. 459.17.)

### Synthesis of Compound 35

In an argon atmosphere, Intermediate 35-c (0.18 g, 0.4 mmol) was dissolved in 5 mL of chloroform, diethyl malonate (0.08 g, 0.5 mmol), 5 mL of a methanol solution, and 1 mL of Et₃N were added thereto, and the mixture was stirred at room temperature for 35 minutes and then concentrated under reduced pressure. The obtained solid was separated and purified by column chromatography utilizing CH₂Cl₂ and hexane to obtain Compound 35 (0.18 g, 0.3 mmol, 59%).

By ESI-LCMS, the compound thus obtained was identified as Compound 35. (ESI-LCMS: [M]⁺: C₃₈H₃₅NO₄S. 601.23.)

### Comparative Example 1

As an anode, a glass substrate with 15 Ω/cm² (1,200 Å) ITO formed thereon (manufactured by Corning Inc.) was cut to a size of 50 mm×50 mm×0.7 mm, sonicated utilizing isopropyl alcohol and pure water, each for 5 minutes, cleaned by irradiation of ultraviolet rays and exposure of ozone thereto for 30 minutes, and then mounted on a vacuum deposition apparatus.

2-TNATA was vacuum-deposited on the anode to form a hole injection layer having a thickness of 100 Å. 4,4′-bis[N-(1-naphthyl)-N-phenylamino]biphenyl (hereinafter, referred to as NPB) was vacuum-deposited on the hole injection layer to form a hole transport layer having a thickness of 1,250 Å.

Subphthalocyanine chloride (hereinafter, referred to as SubPC) was vacuum-deposited on the hole transport layer to form a first layer having a thickness of 200 Å, which was included in a photoactive layer.

Fullerene was vacuum-deposited on the first layer to form a second layer having a thickness of 250 Å, which was included in the photoactive layer.

BAlq was vacuum-deposited on the second layer to form a hole blocking layer having a thickness of 50 Å. ET1 was vacuum-deposited on the hole blocking layer to form an electron transport layer having a thickness of 300 Å. LiQ was vacuum-deposited on the electron transport layer to form an electron injection layer having a thickness of 10 Å. AgMg was vacuum-deposited on the electron injection layer to form a cathode having a thickness of 100 Å, thereby completing the manufacture of an opto-electronic device.

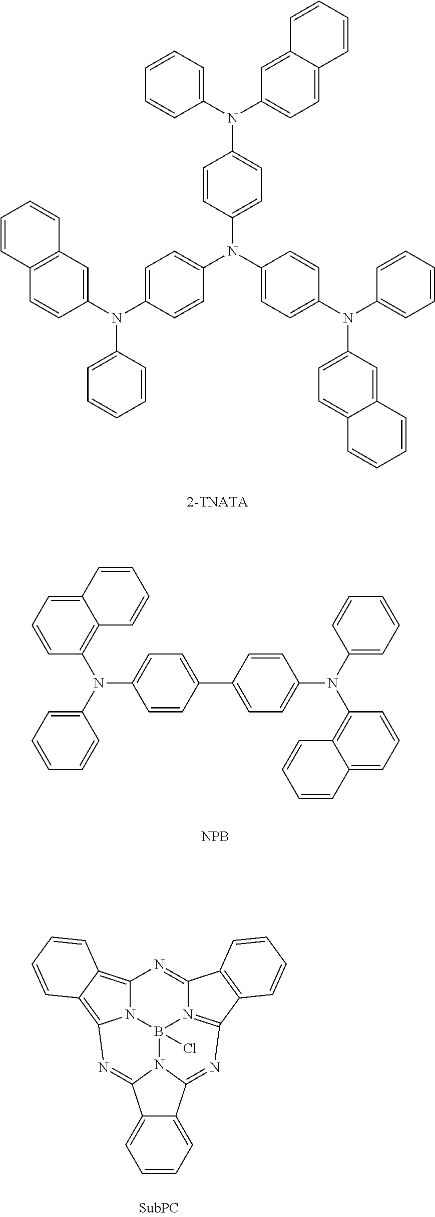

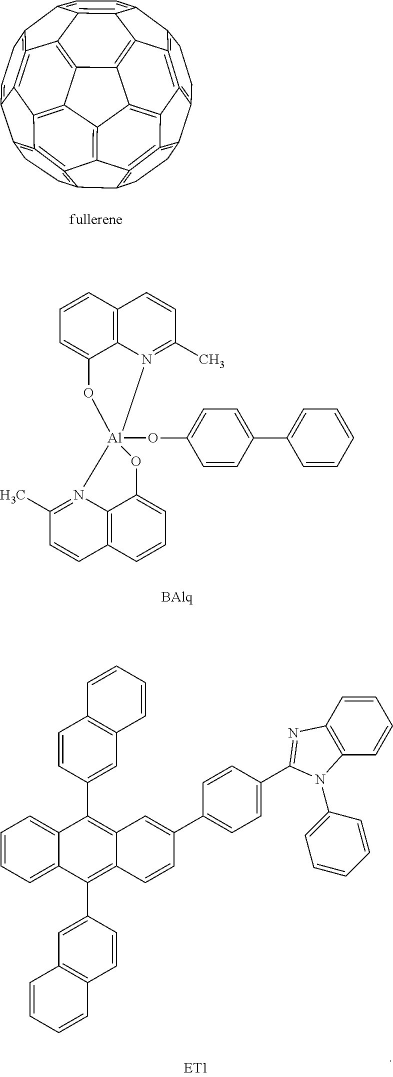

### Comparative Examples 2 to 4 and Examples 1 to 5

Opto-electronic devices were manufactured in substantially the same manner as in Comparative Example 1, except that compounds shown in Table 1 were each utilized instead of SubPC in forming the first layer included in the photoactive layer.

### Evaluation Example 1

To evaluate the characteristics of the compound utilized in the first layer in each of Comparative Examples 1 to 4 and Examples 1 to 5, the HOMO energy level, LUMO energy level, maximum absorption wavelength (λ_{abs}), oscillator strength (OSC), and exciton binding energy (E_{b}) thereof were measured, and the results are shown in Table 1.

In evaluating the above characteristics, quantum simulation was performed at a level of B3LYP/6-311G** based on the time-dependent density functional theory (TD-DFT) method by utilizing the Gaussian program to evaluate the energy levels and oscillator strength in a structurally optimized state and an excited state.

TABLE 1

| | | HOMO | LUMO | | | |
|---|---|---|---|---|---|---|
| | Compound | energy | energy | | | |
| | utilized in first | level | level | λ_{abs} | | E_{b} |
| | layer | (eV) | (eV) | (nm) | OSC | (eV) |
| Comparative | SubPC | −5.55 | −2.83 | 590 | 0.62 | 0.22 |
| Example 1 | | | | | | |
| Comparative | A | −5.62 | −3.02 | 521 | 0.76 | 0.23 |
| Example 2 | | | | | | |
| Comparative | B | −5.83 | −3.06 | 505 | 0.29 | 0.31 |
| Example 3 | | | | | | |
| Comparative | C | −5.46 | −2.60 | 482 | 0.50 | 0.29 |
| Example 4 | | | | | | |
| Example 1 | 2 | −5.47 | −2.94 | 535 | 0.81 | 0.21 |
| Example 2 | 3 | −5.50 | −2.99 | 531 | 0.81 | 0.18 |
| Example 3 | 15 | −5.53 | −2.97 | 528 | 0.88 | 0.21 |
| Example 4 | 33 | −5.58 | −2.98 | 524 | 0.89 | 0.23 |
| Example 5 | 35 | −5.45 | −2.87 | 526 | 0.85 | 0.22 |

From Table 1, it was confirmed that the organic compounds utilized in the first layer in Examples 1 to 5 had i) a maximum absorption wavelength (λ_{abs}) in green light, ii) suitably high oscillator strength (OSC), and iii) suitably low exciton binding energy (E_{b}), as compared with the organic compounds utilized in the first layer in Comparative Examples 1 to 4. Accordingly, it is believed that the organic compounds utilized in the first layer in Examples 1 to 5 i) absorbed green light with high luminescence efficiency, ii) had excellent or suitable characteristics of absorbing green light, and iii) had excellent or suitable characteristics in terms of absorbing light and separating the absorbed light into electrons and holes, as compared with the organic compounds utilized in the first layer in Comparative Examples 1 to 4.

### Evaluation Example 2

To evaluate the characteristics of the opto-electronic devices manufactured in Comparative Examples 1 to 4 and Examples 1 to 5, the external quantum efficiency (EQE) and dark current density (J_{dark}) thereof were measured, and the results are shown in Table 2.

Light (530 nm) was irradiated to an opto-electronic device by utilizing an external quantum efficiency meter (K3100, McScience, Korea). Current generated during light irradiation was measured utilizing an ammeter (Keithley, Tektronix, USA). The external quantum efficiency (EQE) was calculated utilizing the irradiated light and the measured current.

A voltage (−3V) was applied to an anode by utilizing electro-optical characteristic evaluation equipment (K3100, McScience, Korea). Current flowing when the voltage was applied was measured utilizing an ammeter (Keithley, Tektronix, USA). The dark current density (J_{dark}) was calculated utilizing the measured current.

TABLE 2

| | Compound | Compound | | |
|---|---|---|---|---|
| | utilized in | utilized in | EQE | J_{dark} |
| | first layer | second layer | (%) | (mA/cm²) |
| Comparative | SubPC | Fullerene | 20 | 3.5 × 10⁻⁶ |
| Example 1 | | | | |
| Comparative | A | Fullerene | 25 | 5.0 × 10⁻⁶ |
| Example 2 | | | | |
| Comparative | B | Fullerene | 15 | 8.3 × 10⁻⁶ |
| Example 3 | | | | |
| Comparative | C | Fullerene | 18 | 7.8 × 10⁻⁶ |
| Example 4 | | | | |
| Example 1 | 2 | Fullerene | 30 | 3.0 × 10⁻⁶ |
| Example 2 | 3 | Fullerene | 31 | 2.6 × 10⁻⁶ |
| Example 3 | 15 | Fullerene | 36 | 4.2 × 10⁻⁶ |
| Example 4 | 33 | Fullerene | 37 | 4.7 × 10⁻⁶ |
| Example 5 | 35 | Fullerene | 34 | 4.5 × 10⁻⁶ |

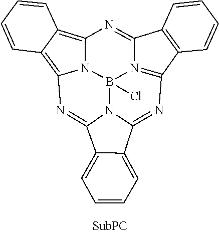

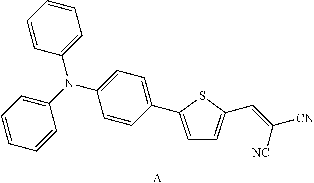

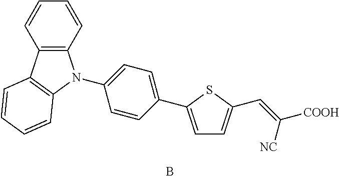

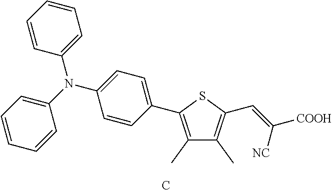

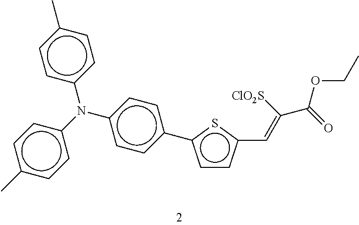

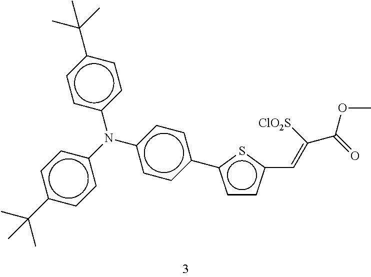

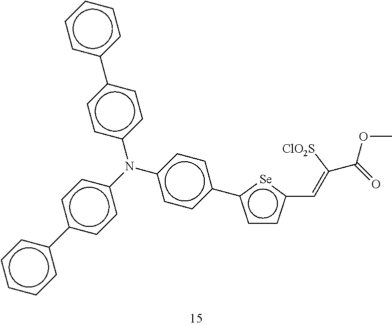

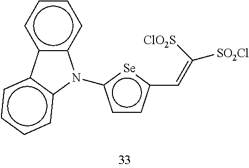

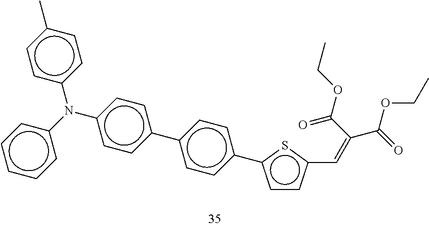

From Table 2, it was confirmed that the opto-electronic devices according to Examples 1 to 5 each had suitably high external quantum efficiency (EQE) and suitably low dark current density (J_{dark}), as compared with the opto-electronic devices according to Comparative Examples 1 to 4. Accordingly, it is believed that the opto-electronic devices according to Examples 1 to 5 had high photoelectric characteristics and low noises, as compared with the opto-electronic devices according to Comparative Examples 1 to 4. For example, it is believed that the opto-electronic devices according to Examples 1 to 5 had excellent or suitable photoelectric characteristics.

According to the one or more embodiments, the organic compound represented by Formula 1 may have excellent or suitable characteristics of absorbing green light and excellent or suitable characteristics in terms of absorbing light and separating the absorbed light into electrons and holes. An opto-electronic device including the organic compound may have excellent or suitable photoelectric characteristics.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope of the present disclosure as defined by the following claims and equivalents thereof.

What is claimed is:

1. An opto-electronic device comprising:
a first electrode;
a second electrode facing the first electrode;
a photoactive layer between the first electrode and the second electrode; and
an organic compound represented by Formula 1: 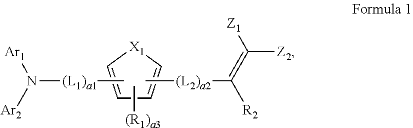
wherein, in Formula 1,
X₁ is O, S, Se, or Te,
L₁ and L₂ are each independently a single bond, a C₁-C₂₀ alkylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
a1 and a2 are each independently an integer from 1 to 5, wherein, when a1 is from 2 to 5, a plurality of L₁(s) are identical to or different from each other, and when a2 is from 2 to 5, a plurality of L₂(s) are identical to or different from each other,
R₁ and R₂ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a3 is an integer from 0 to 2, wherein, when a3 is 2, two R₁(s) are identical to or different from each other,
Z₁ and Z₂ are each independently: hydrogen; deuterium; —F; —Cl; —Br; —I; —CF₃; —C(═O)OR₃; —SO₃H; or —S(═O)₂R₄,
R₃ and R₄ are each independently: hydrogen; deuterium; —F; —Cl; —Br; —I; —CF₃; or a C₁-C₂₀ alkyl group substituted with deuterium, —F, —Cl, —Br, —I, —CF₃, or any combination thereof,
when one of Z₁ or Z₂ is hydrogen and the other one of Z₁ or Z₂ that is not hydrogen is —C(═O)OR₃, R₃ is not hydrogen,
Ar₁ and Ar₂ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
Ar₁ and Ar₂ are optionally bonded to each other via a single bond,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

2. The opto-electronic device of claim 1, wherein the photoactive layer comprises the organic compound.

3. The opto-electronic device of claim 1, further comprising:
a hole transport region between the first electrode and the photoactive layer; and
an electron transport region between the photoactive layer and the second electrode.

4. The opto-electronic device of claim 3, wherein the photoactive layer comprises: a first layer adjacent to the hole transport region; and a second layer adjacent to the electron transport region, and
the first layer comprises the organic compound.

5. The opto-electronic device of claim 4, wherein the photoactive layer further comprises a third layer between the first layer and the second layer, and
the third layer comprises the organic compound.

6. The opto-electronic device of claim 1, wherein the photoactive layer comprises an electron accepting compound.

7. An electronic apparatus comprising the opto-electronic device of claim 1.

8. The electronic apparatus of claim 7, further comprising:
a thin-film transistor electrically connected to the first electrode; and
a color filter, a color conversion layer, a touch screen layer, a polarizing layer, or any combination thereof.

9. An organic compound represented by Formula 1: 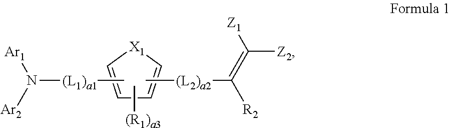
wherein, in Formula 1,
X₁ is O, S, Se, or Te,
L₁ and L₂ are each independently a single bond, a C₁-C₂₀ alkylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
a1 and a2 are each independently an integer from 1 to 5, wherein, when a1 is from 2 to 5, a plurality of Li(s) are identical to or different from each other, and when a2 is from 2 to 5, a plurality of 12(s) are identical to or different from each other,
R₁ and R₂ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₇-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a3 is an integer from 0 to 2, wherein, when a3 is 2, two R₁(s) are identical to or different from each other,
Z₁ and Z₂ are each independently: hydrogen; deuterium; —F; —Cl; —Br; —I; —CF₃; —C(═O)OR₃; —SO₃H; or —S(═O)₂R₄,
R₃ and R₄ are each independently: hydrogen; deuterium; —F; —Cl; —Br; —I; —CF₃; or a C₁-C₂₀ alkyl group substituted with deuterium, —F, —Cl, —Br, —I, —CF₃, or any combination thereof,
when one of Z₁ or Z₂ is hydrogen and the other one of Z₁ or Z₂ that is not hydrogen is —C(═O)OR₃, R³ is not hydrogen,
Ar₁ and Ar₂ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
Ar₁ and Ar₂ are optionally bonded to each other via a single bond,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(021), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

10. The organic compound of claim 9, wherein L₁ and L₂ are each independently selected from among a single bond and groups represented by Formulae 2-1 to 2-3: 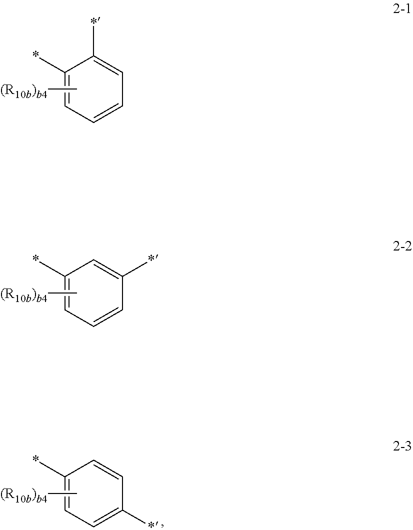
and
wherein, in Formulae 2-1 to 2-3,
definition for R_{10b} is the same as for R₁₀ₐ in Formula 1,
b4 is an integer from 0 to 4,
* indicates a binding site to N in Formula 1, and
*′ indicates a binding site to a corresponding neighboring atom in Formula 1.

11. The organic compound of claim 9, wherein a sum of the number of oxygen atoms in Z₁ and the number of oxygen atoms in Z₂ is 4.

12. The organic compound of claim 9, wherein Z₁ and Z₂ are each independently: —C(═O)OR₃; or —S(═O)₂R₄, and
R₃ and R₄ are each independently: —F; —Cl; —Br; —I; or a C₁-C₁₀ alkyl group substituted with deuterium, —F, —Cl, —Br, —I, —CF₃, or any combination thereof.

13. The organic compound of claim 9, wherein Z₁ and Z₂ are each independently —C(═O)OCH₃, —C(═O)OCH₂CH₃, or —SO₂Cl.

14. The organic compound of claim 9, wherein Ar₁ is a benzene group unsubstituted or substituted with at least one R_{1c},
Ar₂ is a benzene group unsubstituted or substituted with at least one R_{2c}, and
definitions for R_{1c} and R_{2c} are each the same as for R₁₀ₐ in Formula 1.

15. The organic compound of claim 14, wherein R_{1c} and R_{2c} are each independently:
deuterium; a C₁-C₁₀ alkyl group; a C₁-C₁₀ alkoxy group; a phenyl group; a carbazolyl group; or any combination thereof; or
a phenyl group or a carbazolyl group, each substituted with deuterium, a C₁-C₁₀ alkyl group, a C₁-C₁₀ alkoxy group, a phenyl group, a carbazolyl group, or any combination thereof.

16. The organic compound of claim 14, wherein the organic compound is represented by at least one of Formulae 3-1 to 3-5: 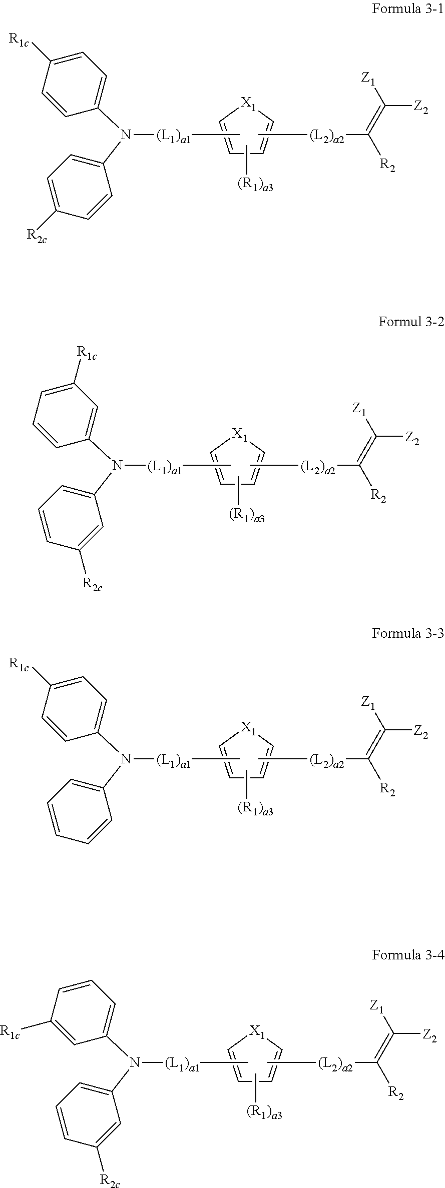 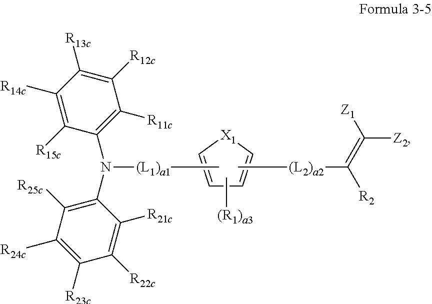
wherein, in Formulae 3-1 to 3-5,
definitions for R_{11c} to R_{15c}, and R_{21c} to R_{25c} are each the same as for R₁₀ₐ in Formula 1.

17. The organic compound of claim 14, wherein the organic compound is represented by at least one of Formulae 4-1 to 4-3: 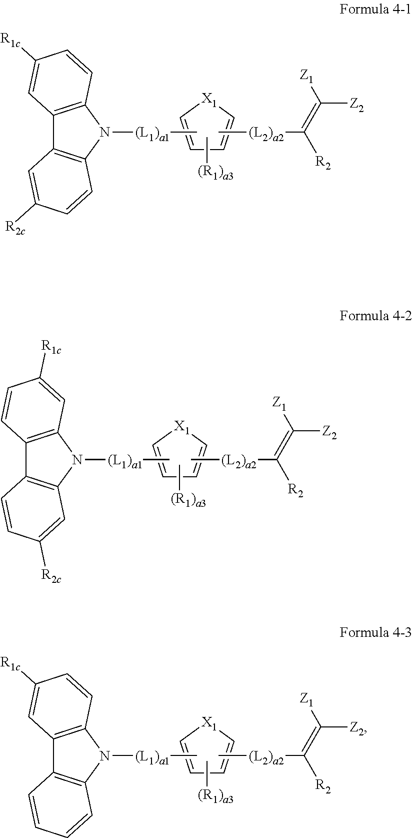
and
wherein, in Formulae 4-1 to 4-3,
definitions for R_{11c} to R_{15c}, and R_{21c} to R_{25c} are the same as for R₁₀ₐ in Formula 1.

18. The organic compound of claim 9, wherein a maximum absorption wavelength of the organic compound is in a range of about 490 nm to about 570 nm.

19. The organic compound of claim 9, wherein an oscillator strength (OSC) of the organic compound is in a range of about 0.8 to about 1.0.

20. The organic compound of claim 9, wherein the organic compound is at least one of Compounds 1 to 40: 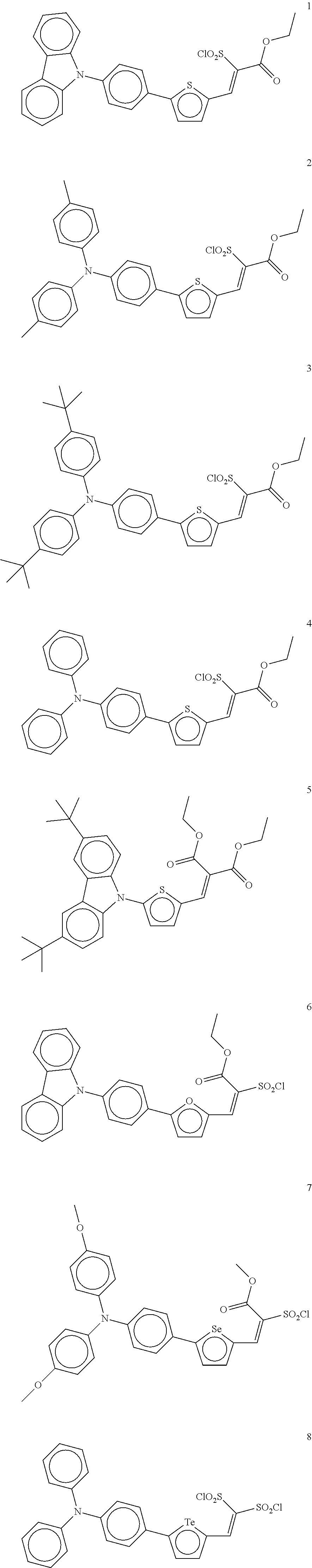 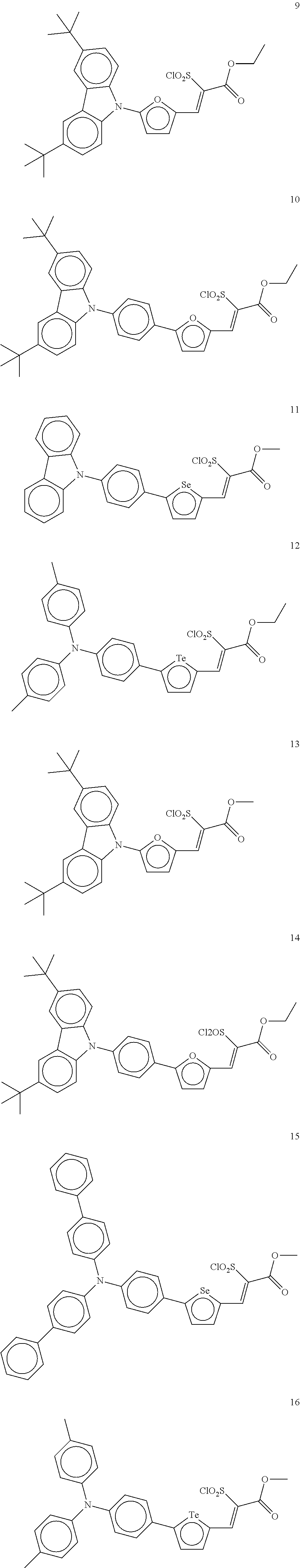 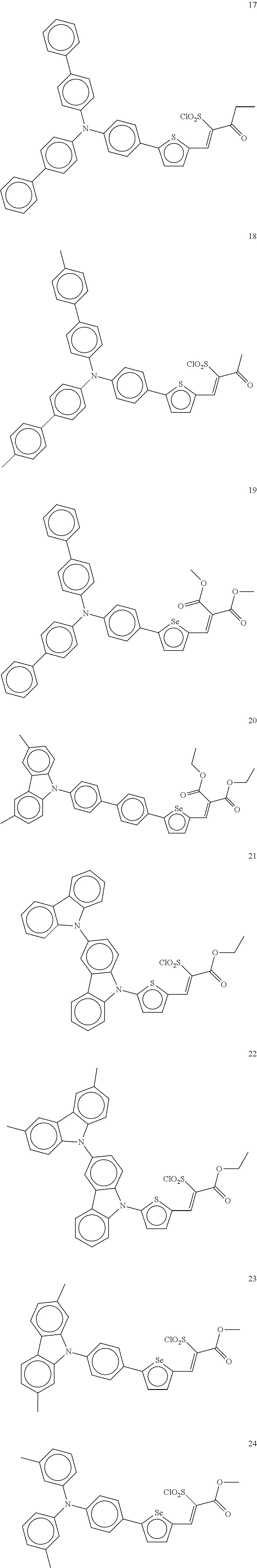 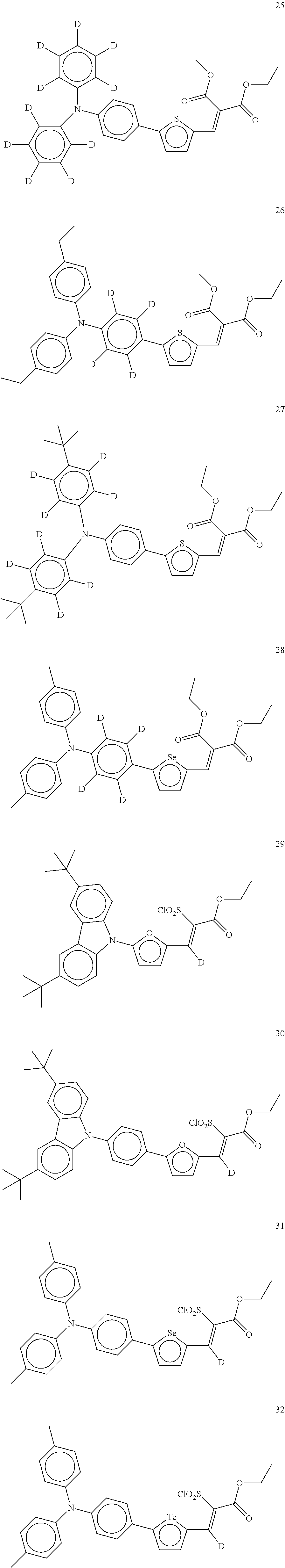 [chemical structure]
